(19) United States
(12) Patent Application Publication
(10) Pub. No.: US 2020/0407303 A1
(43) Pub. Date: Dec. 31, 2020

(54) QUINONES AND PROCESS OF OBTAINING SAME

(71) Applicant: BASF SE, Ludwigshafen am Rhein (DE)

(72) Inventors: Karin SCHEIN-ALBRECHT, Ludwigshafen am Rhein (DE); Wolfgang SIEGEL, Ludwigshafen am Rhein (DE)

(21) Appl. No.: 16/969,970

(22) PCT Filed: Feb. 12, 2019
(86) PCT No.: PCT/EP2019/053400

§ 371 Date: Aug. 13, 2020

(30) Foreign Application Priority Data
Feb. 14, 2018 (EP) 18156771.0

(51) Int. Cl. C07C 46/06; B01J 23/72

(52) U.S. Cl. CPC C07C 46/06; C07D 311/72; B01J 23/72

(57) ABSTRACT

Disclosed is a process for the oxidation of at least one chroman (C1) in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2). A further part of the disclosure is a composition comprising at least one chroman (C1) and/or at least one quinone (C30), a solvent mixture comprising at least two solvents or a C-bearing solvent, a copper catalyst, said copper catalyst exhibiting the oxidation state (+1) or (+2) and a gaseous compound comprising, essentially consisting or consisting of oxygen. A quinone preparation and a process of making same is also part of the invention.

Quinones of the prior art are obtained either by reduction of the corresponding carboxylic acids, esters or amides or by oxidation of alcohols or ethers. However, a main drawback of these reactions is that side products formed cannot be avoided or largely suppressed, thus making synthesis on an industrial scale costly and laborious. Some reactions of the prior art do not only require but also consume catalysts, which need to be continuously supplemented. At the same time, spent catalyst is to be sumptuously removed. Furthermore, simple molecules upon oxidation or reduction behave differently compared to more complex entities, which is to say that reaction conditions used with these simple compounds cannot be directly transferred to reactions involving more sophisticated compounds as raw material.

Attempts were already made to convert α-tocopherol into the corresponding α-tocopherol quinone. However, these attempts suffer from several disadvantages and are not suited to be used on an industrial scale.

Nagata et al. (Chem. Pharm. Bull. 48(1) 71-76 (2000) tried to react α-tocopherol (0.1 mmol) with gaseous oxygen in distilled water containing a solubilizing agent in the presence of 1 to 5 μmoles of a metal salt selected from the group consisting of CuSO₄(NH₄)₂SO₄, Cu(ClO₄)₂, Fe(ClO₄)₃, Ni(ClO₄)₂, Co(ClO₄)₂ and Mn(ClO₄)₂ (cf. procedure). The solubilizing agent is selected from the group of detergents of sodium deoxycholate (DOC), sodium cholate (CO), sodium dodecylsulfate (SDS), dodecyltrimethylammonium bromide (C12-TBr), tetradecyltrimethylammonium bromide (C14-TBr), hexadecyltrimethylammonium bromide (C16-TBr), sodium chenodeoxycholate (ChenoDOC), sodium ursodeoxycholate (UDOC), sodium taurodeoxycholate (TDOC), sodium taurochenodeoxycholate (TchenoDCO), sodium taurocholate (TCO), sodium tauroursodeoxycholate (TUDOC), stearyltrimethylammonium bromide (C18-TBr).

Under these conditions, it is revealed “that Cu²⁺ ion is the most effective catalyst for the formation of 5-formyl-7,8-dimethyltocol (5-FDT)”, not for α-tocopherol quinone. “In addition, it was found that all metal ions used above more or less accelerated the formation of 5-FDT, whereas the yield of α-tocopherol quinone (α-TQ) remained low” (cf. p. 71, results). The consumption of α-tocopherol in the presence of Cu²⁺ is pretty low compared to Co²⁺, Mn²⁺, Fe³⁺, Ni²⁺ or in the absence of any metal catalyst and the formation of α-tocopherol quinone is mediocre when related to reactions realized with Co²⁺ or Fe³⁺ as metal ion (cf. FIG. 1). Thus, according to Nagata, Cu²⁺ cannot be considered to be a good catalyst for selectively oxidizing α-tocopherol into α-tocopherol quinone.

Furthermore, without using a solubilizing agent viz. detergent, the amount of unreacted α-tocopherol is pretty high, viz. said solubilizing agent is mandatory, thus making reaction conditions more complicated. However, only particular solubilizing agents like sodium dodecylsulfate (SDS), sodium cholate (CO) and sodium taurodeoxycholate (TODC) convey Cu²⁺ to promote the formation of α-tocopherol quinone over the formation of 5-FDT (cf. FIG. 1, p. 73, left column, FIG. 2, lower line, right), thus making reaction conditions very peculiar. It was also observed that reaction rates are retarded under high concentration of the solubilizing agent (cf. p. 72, left column), which require accurate dosing means and would make an industrial process time consuming, more sophisticated and thus expensive.

In any oxidation reaction realized in Nagata et al. at least two products form, with 5-FDT being in most cases the predominant one. Mostly many more products are observed, which is to say, the copper-mediated oxidation of tocopherol as disclosed in Nagata et al would not bring the skilled person to a clean or rather clean tocopherol quinone in high yield and with short reaction time, a prerequisite for an industrial production process.

Another attempt of oxidizing α-tocopherol into α-tocopherol quinone is disclosed in WO 2011 139897 A2. They incubate 0,1 g (0.23 mmol) of pure α-tocopherol and 0,1 g of CuCl₂ (0.74 mmol) in 10 ml of methanol for 24 h at ambient temperature on a shaker (cf. page 9, line 12) or the tenfold amount of α-tocopherol and CuCl₂ in 10 ml of methanol and incubate this for 12 hours at room temperature on a shaker. (cf. page 12, line 15, page 15, line 4).

Likewise the process disclosed in the '897 publication suffers from the drawback of not providing α-tocopherol quinone in high yield and purity (cf. page 12, line 23 “Chromatogram (b) shows the formation of oxidation products including a major compound identified by HPLC as TQ by comparing its retention time to that of the commercial α-TQ obtained in the same conditions” and page 15, line 14 “Chromatogram (b) in FIG. 8 shows that CuCl₂ completely oxidized TOH to TQ and other unidentified products”). However, in curve b of FIG. 3 the peak corresponding to α-tocopherol quinone is far from being the major one. Rather a substantial peak can be observed between 3 and 4 min in addition to further earlier eluting peaks. The same holds for curve b in FIG. 8. These results appear to be even more disadvantageous, if one takes into account, the product sample prior to HPLC analysis already being submitted to a first purification step, viz. a filtration on a 0.2 μm nylon filter (cf. page 9, line 15, page 12, line 19). Obtaining so many byproducts even after a first cleaning step and a second chromatographical step, viz. after two consecutive purification steps, requires a supplementary cascade of purification steps to achieve high purity α-tocopherol quinone, thus making the synthesis of said α-tocopherol quinone according to the process of the '897 publication laborious and expensive.

Apparently, it seems to be difficult to properly and selectively oxidize chromans into the corresponding quinones and in particular to convert α-tocopherol into α-tocopherol quinone. This can also be seen in the disclosure of Ito et al. (Tetrahedron Lett., 24(47), 5249-5252 (1983)). Oxidation of the small entity 2,3,6-trimethylphenol 1 into the corresponding quinone with hydrogen peroxide was observed to give high yields only in the presence of ruthenium chloride as catalyst. When approaching the oxidation of compounds simulating the structure of α-tocopherol into the corresponding quinone by means of hydrogen peroxide, yields are not so high anymore and could only be achieved with RuCl₃×3 H₂O as a catalyst (“Similarly, a model compound 4 of vitamin E was easily converted to the corresponding quinone 5 in an 80% yield.”, cf. page 5252, I. 6 to 7). These results reflect two things. First the skilled person is not in a position to transfer the teaching of oxidizing small molecules with a catalyst towards larger entities exhibiting very different solubility patterns. Second, with molecules simulating the raw material α-tocopherol even with RuCl₃×3 H₂O as catalyst employed, the yield does not exceed 80%. This is still to be improved for an industrial process. In addition RuCl₃×3 H₂O is a quite expensive catalyst which would make production of chromans by oxidation cost intensive and is not acceptable as industrial standard.

According to all this it is an object of the present invention to overcome the drawbacks of the prior art and to devise a process for the selective oxidation of chromans into the corresponding quinones. Said process shall avoid generating byproducts as best as possible. The quinones formed shall if at all only contain low amounts of inventive process reagents or components thereof. The process shall be fast, cost-effective and simple to be realized. Said process shall be such that it can be implemented into an industrial production and scaled up accordingly. Cleaning, separating or purification steps shall be reduced to the utmost extent possible and preferably are to be avoided completely.

Another object of the invention is to provide a composition containing at least one chroman which is adapted to convert or to be converted into a composition containing the corresponding quinone(s). Said object consequently also comprises a composition comprising at least one quinone, said quinone being obtained from the chroman containing composition by the inventive process for the selective oxidation of chromans.

Yet another object of the invention is a process of obtaining a quinone preparation. Said process shall be simple to realize and thus cost effective. It shall be applicable with any kind of composition containing at least one quinone obtained from the process for the selective oxidation of chromans. Said process for obtaining a quinone preparation shall put the skilled person in a position to simultaneously have an impact on the concentration of different components of the composition containing at least one quinone. To express it with different words, said process of obtaining a quinone preparation shall be designed such that it puts the skilled person in a position to tailor-made modify the amount of trace components, like e.g. inventive process reagents or components thereof, in the quinone preparation. The process of obtaining a quinone preparation in one embodiment shall be shaped to recover or to recycle components or trace components in a purity sufficient to reuse them in the process for the selective oxidation of chromans.

An additional object of the invention is to provide a quinone preparation. Said quinone preparation shall be adapted to satisfy demands of purity and of a trace amount spectrum as required by the feed, the dietary supplement or the pharmaceutical industry. This demand of purity and of reduced amount of trace compounds shall also take into account traces of inventive process reagents or metabolites thereof.

All these objects can be addressed with a process for the oxidation of at least one chroman C1

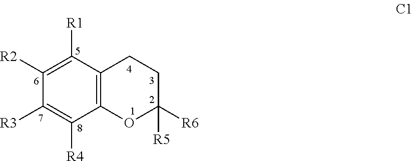

with R1, R3, R4, R5 being H or CH₃, R2 being OH, OAc, OCO—C₁-C₁₈-alkyl, and R6 being alkyl, alkenyl, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2).

As can be seen from the examples below, high yields of quinone C30 are obtained with this process while simultaneously reducing or completely suppressing the amount of side products. A considerable peak as observed in FIG. 3 and FIG. 8 of the '897 paper cannot be observed thus making this process straight forward and cost-effective. Solubilizing agents (detergents) as required in the aqueous reaction solutions of Nagata et al. are not required anymore. This makes the inventive process less complicated and prevents the formation of side products like 5-FDT as disclosed in Nagata et al., Due to its simplicity, the inventive process can be readily scaled up and used on an industrial scale.

Chroman C1 also named 2,3-dihydro-4H-benzopyran or 3,4-dihydro-2H-1-benzopyran within this disclosure is understood to be at least one molecule of formula C1

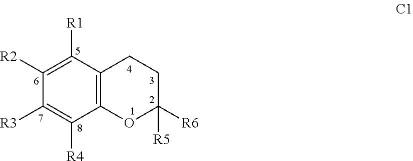

with R1, R3, R4, R5 being H or CH₃, R2 being OH, OAc, OCO—C₁-C₁₈-alkyl, and R6 being alkyl, alkenyl.

Alkyl means C₁₀-C₂₀-alkyl, preferably C₁₄-C₁₈-alkyl and mostly preferred C₁₆-alkyl, viz entities comprising the given number of carbon atoms.

In one embodiment alkyl with respect to R6 is understood to have the structure of formula C2

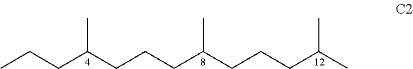 [SMILES: CCCC(C)CCCC(C)CCCC(C)C]

with the stereocenters in position 4, 8 having a 4R,8R-configuration, a 4R,8S-configuration, a 4S,8R-configuration or a 4S,8S-configuration.

In one embodiment the chroman C1 is α-tocopherol of formula C3

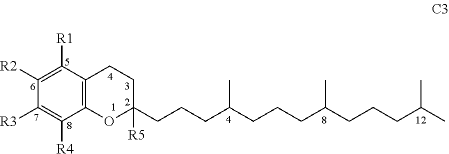

with R1, R3, R4, R5 being CH₃, R2 being OH, OAc, OCO—C₁-C₁₈-alkyl, with the stereocenters of the lateral chain in position 4,8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is α-tocopherol of formula C4

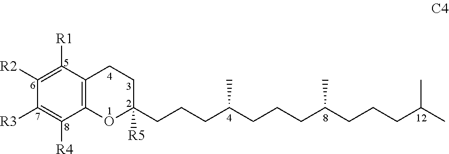

with R1, R3, R4, R5 being CH₃, R2 being OH, OAc, OCO—C₁-C₁₈-alkyl, and with the stereocenter at C2 of the annular moiety as well as the stereocenters of the lateral chain in position 4, 8 having a R-configuration.

In one embodiment the chroman C1 is α-tocopherol of formula C5

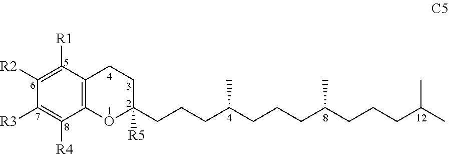

with R1, R3, R4, R5 being CH₃, R2 being OH and with the stereocenter at C2 of the annular moiety as well as the stereocenters of the lateral chain in position 4, 8 having a R-configuration.

In one embodiment the chroman C1 is β-tocopherol of formula C6

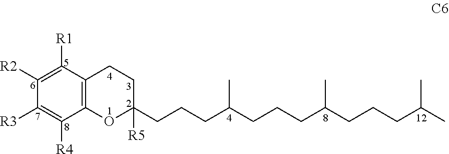

with R1, R4, R5 being CH₃, R2 being OH, OAc, OCO—C₁-C₁₈-alkyl, R3 being H, with the stereocenters of the lateral chain in position 4, 8 having a 4R,8R-configuration, a 4R,8S-configuration, a 4S,8R-configuration or a 4S,8S-configuration and the stereocenter at C2 of the annular moiety having a R or S configuration.

In one embodiment the chroman C1 is γ-tocopherol of formula C7

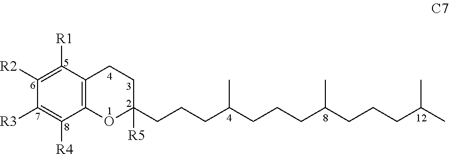

with R1 being H, R3, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is 5-tocopherol of formula C8

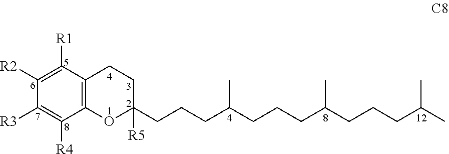

with R1, R3 being H, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

Alkenyl means C₁₀-C₂₀-alkenyl, preferably C₁₄-C₁₈-alkenyl and mostly preferred C₁₆-alkenyl, viz entities comprising the given number of carbon atoms and having at least one double bond.

In one embodiment alkenyl is understood to have the structure of formula C9

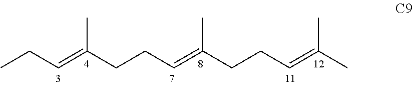 [SMILES: CC/C=C(\C)CC/C=C(\C)CCC=C(C)C]

with the methyl groups in position 4, 8 having
a 4 cis, 8 cis-conformation,
a 4 cis, 8 trans-conformation,
a 4 trans, 8 cis-conformation or
a 4 trans, 8 trans-conformation
   and the double bounds in position 3 and 7 having
a 3E,7E-configuration,
a 3E,7Z-configuration,
a 3Z,7E-configuration or
a 3Z,7Z-configuration.

In one embodiment alkenyl is understood to have the structure of formula C10

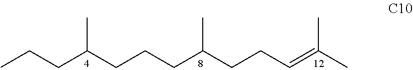 [SMILES: CCCC(C)CCCC(C)CCC=C(C)C]

with the stereocenters in position 4, 8 having a 4R,8R-configuration, a 4R,8S-configuration, a 4S,8R-configuration or a 4S,8S-configuration.

In one embodiment alkenyl is understood to have the structure of formula C11

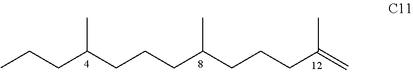 [SMILES: C=C(C)CCCC(C)CCCC(C)CCC]

with the stereocenters in position 4, 8 having a 4R,8R-configuration, a 4R,8S-configuration, a 4S,8R-configuration or a 4S,8S-configuration.

In one embodiment the chroman C1 is α-tocotrienol of formula C12

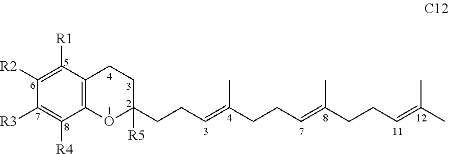

with R1, R3, R4, R5 being CH₃, R2 being OH, OAc, OCO—C₁-C₁₈-alkyl, the stereocenter at C2 of the annular moiety having a R or S configuration and the methyl groups in the exocyclic position 4, 8 having
a 4 cis, 8 cis-conformation,
a 4 cis, 8 trans-conformation,
a 4 trans, 8 cis-conformation or
a 4 trans, 8 trans-conformation
   and the double bounds in exocyclic position 3 and 7 having
a 3E,7E-configuration,
a 3E,7Z-configuration,
a 3Z,7E-configuration or
a 3Z,7Z-configuration.

In one embodiment the chroman C1 is α-tocotrienol of formula C13

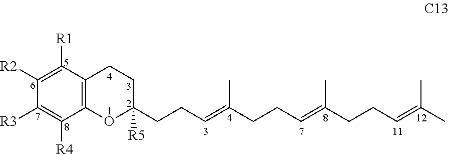

with R1, R3, R4, R5 being CH₃, R2 being OH, OAc, OCO—C₁-C₁₈-alkyl, and with the stereocenter at C2 of the annular moiety having a R configuration and the methyl groups in the exocyclic position 4, 8 having
a 4 cis, 8 cis-conformation,
a 4 cis, 8 trans-conformation,
a 4 trans, 8 cis-conformation or
a 4 trans, 8 trans-conformation
   and the double bounds in exocyclic position 3 and 7 having
a 3E,7E-configuration,
a 3E,7Z-configuration,
a 3Z,7E-configuration or
a 3Z,7Z-configuration.

In one embodiment the chroman C1 is α-tocotrienol of formula C14

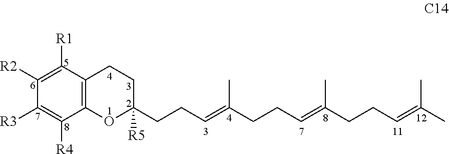

with R1, R3, R4, R5 being CH₃, R2 being OH and with the stereocenter at C2 of the annular moiety having a R-configuration and the methyl groups in the exocyclic position 4, 8 having
a 4 cis, 8 cis-conformation,
a 4 cis, 8 trans-conformation,
a 4 trans, 8 cis-conformation or
a 4 trans, 8 trans-conformation
   and the double bounds in exocyclic position 3 and 7 having
a 3E,7E-configuration,
a 3E,7Z-configuration,
a 3Z,7E-configuration or
a 3Z,7Z-configuration.

In one embodiment the chroman C1 is β-tocotrienol of formula C15

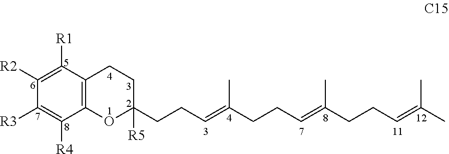

with R1, R4, R5 being CH₃, R2 being OH, OAc, OCO—C₁-C₁₈-alkyl, R3 being H, the stereocenter at C2 of the annular moiety having a R or S configuration and the methyl groups in the exocyclic position 4, 8 having
a 4 cis, 8 cis-conformation,
a 4 cis, 8 trans-conformation,
a 4 trans, 8 cis conformation or
a 4 trans, 8 trans-conformation.
   and the double bounds in exocyclic position 3 and 7 having
a 3E,7E-configuration,
a 3E,7Z-configuration,
a 3Z,7E-configuration or
a 3Z,7Z-configuration.

In one embodiment the chroman C1 is γ-tocotrienol of formula C16

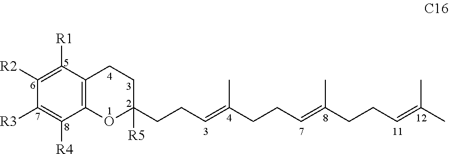

with R1 being H, R3, R4, R5 being CH₃, R2 being OH, OAc, OCO—C₁-C₁₈-alkyl, the stereocenter at C2 of the annular moiety having a R or S configuration and the methyl groups in the exocyclic position 4, 8 having
a 4 cis, 8 cis-conformation,
a 4 cis, 8 trans-conformation,
a 4 trans, 8 cis-conformation or
a 4 trans, 8 trans-conformation
   and the double bounds in exocyclic position 3 and 7 having
a 3E,7E-configuration,
a 3E,7Z-configuration,
a 3Z,7E-configuration or
a 3Z,7Z-configuration.

In one embodiment the chroman C1 is 5-tocotrienol of formula C17

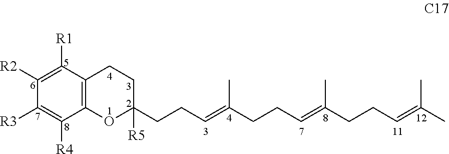

with R1, R3 being H, R4, R5 being CH₃, R2 being OH, OAc, OCO—C₁-C₁₈-alkyl, the stererocenter at C2 of the annular moiety having a R or S configuration and the methyl groups in the exocyclic position 4, 8 having
a 4 cis, 8 cis-conformation,
a 4 cis, 8 trans-conformation,
a 4 trans, 8 cis-conformation or
a 4 trans, 8 trans-conformation
   and the double bounds in exocyclic position 3 and 7 having
a 3E,7E-configuration,
a 3E,7Z-configuration,
a 3Z,7E-configuration or
a 3Z,7Z-configuration.

In one embodiment the chroman C1 is α-tocomonoenol of formula C18

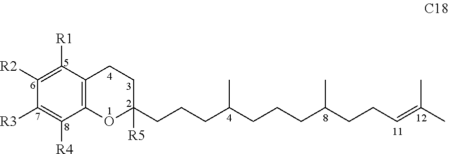

with R1, R3, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is α-tocomonoenol of formula C19

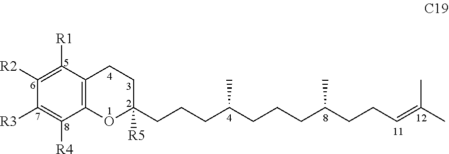

with R1, R3, R4, R5 being CH₃, R2 being OH, OAc, OCO—C₁-C₁₈-alkyl, and with the stereocenter at C2 of the annular moiety as well as the stereocenters of the lateral chain in position 4, 8 having a R-configuration.

In one embodiment the chroman C1 is α-tocomonoenol of formula C20

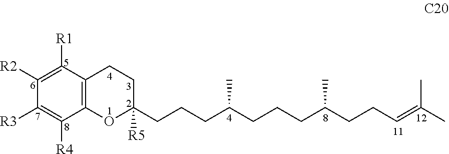

with R1, R3, R4, R5 being CH₃, R2 being OH, and with the stereocenter at C2 of the annular moiety as well as the stereocenters of the lateral chain in position 4,8 having a R-configuration.

In one embodiment the chroman C1 is β-tocomonoenol of formula C21

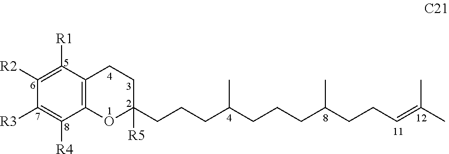

with R1, R4, R5 being CH₃, R2 being OH, OAc, OCO—C₁-C₁₈-alkyl, R3 being H, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is γ-tocomonoenol of formula C22

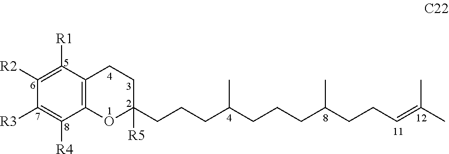

with R1 being H, R3, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is δ-tocomonoenol of formula C23

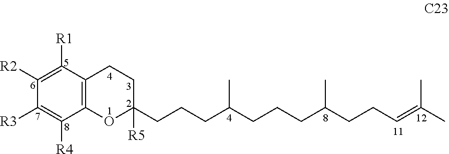

with R1, R3 being H, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is a marine-derived α-tocopherol (α-MDT) of formula C24

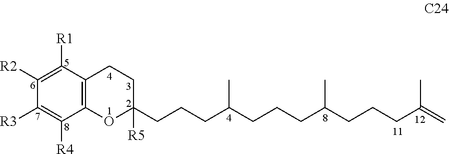

with R1, R3, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is a marine-derived α-tocopherol (α-MDT) of formula C25

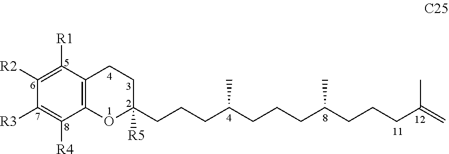

with R1, R3, R4, R5 being CH₃, R2 being OH, OAc, OCO—C₁-C₁₈-alkyl, and with the stereocenter at C2 of the annular moiety as well as the stereocenters of the lateral chain in position 4,8 having a R-configuration.

In one embodiment the chroman C1 is a marine-derived α-tocopherol (α-MDT) of formula C26

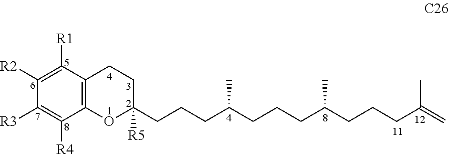

with R1, R3, R4, R5 being CH₃, R2 being OH, and with the stereocenter at C2 of the annular moiety as well as the stereocenters of the lateral chain in position 4,8 having a R-configuration.

In one embodiment the chroman C1 is a marine-derived β-tocopherol (β-MDT) of formula C27

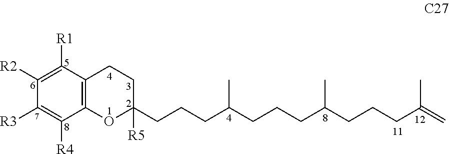

with R1, R4, R5 being CH₃, R2 being OH, OAc, OCO—C₁-C₁₈-alkyl, R3 being H, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is a marine-derived γ-tocopherol (γ-MDT) of formula C28

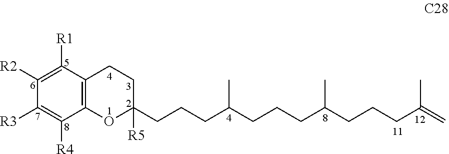

with R1 being H, R3, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is a marine-derived 5-tocopherol (5-MDT) of formula C29

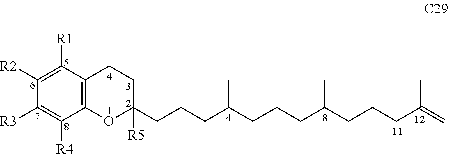

with R1, R3 being H, R4, R5 being CH₃, R2 being OH, OAc, OCO—C₁-C₁₈-alkyl, with the stereocenters of the lateral chain in position 4, 8 having a 4R,8R-configuration, a 4R,8S-configuration, a 4S,8R-configuration or a 4S,8S-configuration, and the stereocenter at C2 of the annular moiety having a R or S configuration.

In one embodiment the chroman C1 is a mixture of at least two of the embodiments C3, C4, C5, C6, C7, C8, C12, C13, C14, C15, C16, C17, C18, C19, C20, C21, C22, C23, C24, C25, C26, C27, C28, C29.

The inventive solvent mixture comprising at least two solvents in one embodiment is a solvent mixture made of a polar solvent and a non-polar solvent.

In a preferred embodiment, the solvent mixture comprising at least two solvents is a mixture of water and another solvent. Said other solvent is selected from the group consisting of alcohols, diols, aliphatic hydrocarbons, aromatic hydrocarbons, ethers, glycolethers, polyethers, polyethylene glycol, ketones, esters, amides, nitriles, halogenated solvents, carbonates, dimethyl sulfoxide and sulfolane.

Said other solvent in a further developed embodiment almost does not mix with water, preferably does not mix at all with water.

The term alcohol within this invention comprises at least one primary, secondary or tertiary alcohol having from 1 to 18 carbon atoms, preferably at least one saturated primary, secondary or tertiary alcohol having from 1 to 18 carbon atoms.

Said at least one, preferably saturated, primary, secondary or tertiary alcohol having from 1 to 18 carbon atoms is selected from the group consisting of methanol, ethanol, propanol, isopropanol, 1-butanol, 2-butanol, 2-methyl-1-propanol, tert-butyl alcohol, pentanol in all its isomeric forms, for example 1-pentanol or n-pentanol or n-amyl alcohol, 3-methylbutan-1-ol or isoamyl alcohol, 2-methyl-1-butanol, 2.2-dimethylpropan-1-ol, 2-pentanol, 3-pentanol, 3-methyl-2-butanol, 2-methyl-2-butanol, cyclopentanol, hexanol in all its isomeric forms, for example 1-hexanol or n-hexanol, cyclohexanol, 2-methyl-1-pentanol, 3-methyl-1-pentanol, 4-methyl-1-pentanol, 2,2-dimethyl-1-butanol, 1,3-dimethyl-1-butanol, 2,3-dimethyl-1-butanol, 3,3-dimethyl-1-butanol, 2-ethylbutan-1-ol, 2-hexanol, 3-hexanol, 3-methyl-2-pentanol, 4-methyl-2-pentanol, 3,3-dimethyl-2-butanol, 2-methyl-2-pentanol, 3-methylpentan-3-ol, 2,3-dimethyl-2-butanol, methyylcyclopentanol, heptanol in all its isomeric forms, for example 1-heptanol, 2-heptanol, 3-heptanol, 3-ethyl-3-pentanol, octanol in all its isomeric forms, for example 1-octanol, 2-octanol, 2-ethylhexanol, 1-nonanol, 1-decanol, 2-ethyl-1-hexanol, 1-dodecanol, 1-tetradecanol, 1-hexadecanol, 1-octadecanol.

Higher primary, secondary or tertiary alcohols were shown to be less sensitive towards ignition, which is preferred for the inventive process working under or with a gaseous compound comprising or consisting of oxygen. In addition, they scarcely or not at all mix with water, thus they can be easily separated from an aqueous fraction.

In a preferred embodiment of the invention alcohol therefore is understood to be at least one primary, secondary or tertiary alcohol having from 5 to 18 carbon atoms, preferably at least one saturated primary, secondary or tertiary alcohol having from 5 to 18 carbon atoms.

In another preferred embodiment of the invention alcohol is understood to be at least one primary, secondary or tertiary alcohol having from 6 to 18 carbon atoms, preferably at least one saturated primary, secondary or tertiary alcohol having from 6 to 18 carbon atoms.

For its ease of availability, alcohol is at least one primary, secondary or tertiary alcohol having from 5 to 8 carbon atoms, preferably at least one saturated primary, secondary or tertiary alcohol having from 5 to 8 carbon atoms.

Availability revealed said alcohol being at least one, preferably saturated, primary, secondary or tertiary alcohol being selected from the group consisting of 1-pentanol, 1-hexanol or n-hexanol, 2-ethylhexanol, 3-heptanol, 2-octanol, 3-ethyl-3-pentanol, 1,3-dimethyl butanol or amylmethyl alcohol, diacetone alcohol, methylisobutyl carbinol or 4-methyl-2-pentanol, tert.-hexyl alcohol, cyclohexanol, 1,6-hexanediol, 1,5 hexanediol, 1,4-hexanediol, 1,3-hexanediol, 2-methyl-2,4-pentanediol, pinacol or 2,3-dimethyl-2,3-butanediol, 1,2,5-hexanetriol, 1,2,6-hexanetriol, trimethylolpropane.

Another aspect of the inventive process focuses on low amounts of inventive process reagents or components thereof to be associated with the quinones formed. This can be promoted or achieved with a special type of alcohol used. In a preferred embodiment of the invention alcohol therefore is understood to be at least one secondary or tertiary alcohol having from 5 to 18 carbon atoms, preferably at least one saturated secondary or tertiary alcohol having from 5 to 18 carbon atoms.

A valuable embodiment of the invention thus is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) with the solvent mixture comprising at least two solvents being a mixture of water and at least one primary, secondary or tertiary alcohol having from 5 to 18 carbon atoms, preferably at least one saturated secondary or tertiary alcohol having from 5 to 18 carbon atoms.

A further elaborated valuable embodiment of the invention thus is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) with the solvent mixture comprising at least two solvents being a mixture of water and at least one secondary or tertiary alcohol having from 5 to 18 carbon atoms, preferably at least one saturated secondary or tertiary alcohol having from 5 to 18 carbon atoms.

Diol of this disclosure is understood to be at least one compound selected from the group consisting of 1,2-ethanediol or ethylene glycol, 1,2-propanediol, 1,3-propanediol, 1,2-butanediol, 2,3.butanediol, 1,3-butanediol, 2-methyl-1,2-prropanediol, 1,4-butanediol, 1,2-pentanediol, 1,3-pentanediol, 1,4-pentanediol, 1,5-pentanediol, 2,2-dimethyl-3-propanediol, 3-methyl-2,4-pentanediol, 4-hydroxy-4-methyl-2-pentanol, 1,6-hexane diol, 1,5 hexane diol, 1,4-hexane diol, 1,3-hexane diol, 2-methyl-2,4-pentane diol, pinacol, 2,3-dimethyl-2,3-butane diol, diethylene glycol, triethylene glycol, glycerol, 1,2-butylene glycol, 1,2,3-butanetriol, 1,2,4-butanetriol, 2-methyl-2,3-butanediol.

Aliphatic hydrocarbon of this disclosure is understood to be selected from the group consisting of n-pentane, iso-pentane, neo-pentane, n-hexane, hexane in all its isomeric forms, n-heptane, heptane in all its isomeric forms, cyclopentane, cyclohexane, cycloheptane, methyl cyclohexane, octane in all its isomeric forms, nonane in all its isomeric forms, decane in all its isomeric forms, undecane in all its isomeric forms, dodecane in all its isomeric forms, polyethylene and nitromethane.

Aromatic hydrocarbon within the content of this disclosure is understood to be selected form the group consisting of benzene, toluene, xylene in all its isomeric forms, e.g. o-, m-, or p-xylene, ethylbenzene 1,3,5-trimethylbenzene, isopropyl benzene, diisopropyl benzene in all its isomeric forms, 2-isopropyltoluene, 3-isopropyltoluene, 4-isopropyltoluene and nitrobenzene.

Ether within the content of this disclosure is understood to be selected form the group consisting of dimethyl ether, diethyl ether, di-n-propyl ether, diisopropyl ether, methyl ethyl ether, dibutyl ether, dipentyl ether, diisopentyl ether, n-butyl methyl ether, sec-butyl methyl ether, tert-butyl methyl ether, tert-butyl ethyl ether, methyl isobutyl ether, tetrahydrofuran, 2-methyltetrahydrofuran, 3-methyltetrahydrofuran, 2,5-Dimethyltetrahydrofuran, 1,3-dioxolane, tetrahydropyran, 1,4-dioxane, 1,3,5-trioxane, benzylethylether, cyclopentyl methyl ether and anisole.

Glycol ether or polyether within the content of this disclosure is understood to be selected form the group consisting of dimethoxymethane, diethoxymethane, ethylene glycol monomethyl ether, ethylene glycol dimethyl ether, ethylene glycol monoethyl ether, ethylene glycol diethyl ether, ethylene glycol monoisopropyl ether, dipropylene glycole, ethylene glycol monobutyl ether, ethylene glycol dibutyl ether, diethylene glycol monomethyl ether, diethylene glycol dimethyl ether, diethylene glycol monoethyl ether, diethylene gylcol diethyl ether, diethylene glycol diacetate, triethylene glycol dimethyl ether, triethylene glycol diethyl ether, tetramethylene glycol dimethyl ether, polyethylene glycol, 2-methoxy-1-propanol.

Ketone within the content of this disclosure is understood to be selected form the group consisting of acetone, methyl ethyl ketone, diethyl ketone, methyl isopropyl ketone, diisopropyl ketone, methyl isobutyl ketone, cyclopropyl methyl ketone, methyl tert-butyl ketone, 2-pentanone, cyclopentanone, 2-hexanone, cyclohexanone, 2-heptanone, 4-heptanone.

Ester within the content of this disclosure is understood to be selected form the group consisting of methyl formate, methyl acetate, ethyl acetate, n-propyl acetate, isopropyl acetate, n-butyl acetate, tert-butyl acetate, hexyl acetate, methyl propionate, γ-butyrolactone, benzoic acid ethylester, glycol diacetate and diethylene glycol diacetate.

Amide within the content of this disclosure is understood to be selected form the group consisting of N-methylformamide, N,N-dimethylformamide, N-methylacetamide, N,N-dimethylacetamide, N,N-diethylacetamide, N,N-dimethylpropionamide, N,N-dibutylformamide. N-methylpyrrolidone.

Nitrile within the content of this disclosure is understood to be selected form the group consisting of acetonitrile, propionitrile, benzonitrile and trimethylacetonitrile.

Halogenated solvent within the content of this disclosure is understood to be selected form the group consisting of methylene chloride, chloroform, carbon tetrachloride, 1,1-dichloroethylene, 1,2-dichloroethane, 1,1,1,-trichloroethane, 1,1,1,2-tetrachloroethane, 1,1,2,2-tetrachloroethane, chlorobenzene, o-dichlorobenzene, m-dichlorobenzene, p-dichlorobenzene, 1,2,3-trichlorobenzene, 1,2,4-trichlorobenzene, 4-chlorotoluene, trichloroacetonitrile, 2-chloroethanol, 2,2,2-trichloroethanol, 1-chloro-2-propanol, 2,3-dichloropropanol, 2-chloro-1-propanol in all isomeric forms, benzotrichloride, fluorobenzene, difluorobenzene in all its isomeric forms, 2,4,6-trifluorotoluene, 2-fluorobutanol, benzotrifluoride.

Carbonate within the content of this disclosure is understood to be selected form the group consisting of ethylene carbonate, propylene carbonate, dimethyl carbonate or diethyl carbonate.

A C-bearing solvent of the inventive process is any solvent adapted to largely solubilize or entirely solubilize all of the reagents chroman C1, gaseous compound comprising, essentially consisting of, or consisting of oxygen and copper catalyst. Such C-bearing solvent is to have both a hydrophilic character and a lipophilic character.

Such C-bearing solvent is selected from at least one of the group consisting of low aliphatic alcohols, namely from at least one C1-C8-alcohol including C1-C8 diols and C1-C8-triols, N,N-dimethylformamide, N,N-diethylformamide, N-methylpyrrolidone, ethylene carbonate, propylene carbonate, glycol ethers.

C1-C8-alcohols are selected from the group consisting of methanol, ethanol, n-propanol, isopropyl alcohol, n-butanol, sec-butyl alcohol, isobutyl alcohol, tert-butyl alcohol, 1-pentanol, isoamyl alcohol, 2-methyl-1-butanol, neopentyl alcohol, 2-pentanol, 3-pentanol, 3-methyl-2-butanol, 2-methyl-2-butanol, cyclopentanol, n-hexanol (1-hexanol), 2-methyl-1-pentanol, 3-methyl-1-pentanol, 4-methyl-1-pentanol, 2,2-dimethyl-1-butanol, 2,3-dimethyl-1-butanol, 3,3-dimethyl-1-butanol, 2-ethylbutan-1-ol, 2-hexanol, 3-hexanol, 3-methyl-2-pentanol, 4-methyl-2-pentanol in all isomeric forms, 3,3-dimethyl-2-butanol, 2-methyl-2-pentanol, 3-methylpentan-3-ol, 2,3-dimethyl-2-butanol, cyclohexanol, methylcyclopentanol, 1,3-dimethyl butanol, amylmethyl alcohol, methylisobutyl carbinol, 4-methyl-2-pentanol, tert-hexyl alcohol, n-heptanol, 2-heptanol, 3-heptanol, 3-ethyl-3-pentanol, n-octanol or 1-octanol, 2-octanol, 2-ethylhexanol, 1,2-ethanediol, 1,2-propanediol, 1,3-propanediol, 1,2-butanediol, 1,3 butanediol, 2,3-butanediol, 2-methyl-1,2-propanediol, 1,4-butanediol, 1,2-pentanediol, 1,3-pentanediol, 1,4-pentanediol, 1,5-pentanediol, 2,4-pentanediol, 2,2-dimethyl-1,3-propanediol, 3-methyl-2,4-pentanediol, 4-hydroxy-4-methyl-2-pentanol, 1,2,4-trihydroxybutane, 1,2,3-trihydroxybutane, triethyleneglycol, 1,6-hexane diol, 1,5 hexane diol, 1,4-hexane diol, 1,3-hexane diol, 2-methyl-2,4-pentane diol, pinacol, 2,3-dimethyl-2,3-butandiol, 1,2,5-hexane triol, 1,2,6-hexane triol, 2-methyl-2,3-butandiol, ethylene glycol monomethyl ether, ethylene glycol monoethyl ether, 2-ethoxyethanol, ethylene glycol monobutyl ether, 2-isopropoxyethanol, diethylene glycol, diethylene glycol monomethyl ether, diethylene glycol monoethyl ether, diethylene glycol diacetate, propylene glycol, 1,2-butylene glycol, triethylene glycol, glycerol, glycol diacetate and diethylene glycol diacetate, 2-methoxy-1-propanol.

Glycol ethers are for example ethylene glycol dimethyl ether, ethylene glycol diethyl ether, ethylene glycol dibutyl ether, diethylene glycol dimethyl ether, diethylene glycol diethyl ether, dipropylene glycole, trimethylene glycol dimethyl ether, trimethylene glycol diethyl ether, triethylene glycol dimethylether.

The gaseous compound within this invention is any compound fulfilling the requirements of being gaseous at the reaction temperature of the inventive process and containing at least one oxygen atom. Said gaseous compound in one embodiment is selected from the group consisting of oxygen in the singlet or triplet state, ozone, air, lean air, gaseous hyperoxide, gaseous peroxide, a mixture of an inert gas and oxygen with the amount of oxygen ranging from 1 vol % to 100 vol %, including a mixture of oxygen and nitrogen, preferably, it is selected from air, lean air and oxygen in the triplet state and mostly preferred from air and lean air.

A copper catalyst exhibiting the oxidation state (+1) or (+2) is any chalcogenic or halogenic copper compound. A copper catalyst of the invention is selected from the group consisting of CuCl₂×2 H₂O, CAS no: 10125-13-0; CuCl₂, CAS no: 7447-39-4; CuCl, CAS no: 7758-89-6; CuCl₂×2 H₂O combined with LiCl, CAS no: 7447-39-4 or combined with LiCl×2H₂O, CAS no: 10125-13-0; CuCl₂×2 H₂O combined with MgCl₂, CAS no: 7786-30-3 or with MgCl₂×6 H₂O 7791-18-6; CuSO₄×5 H₂O, CAS no: 10257-54-2; Cu(II)-trifluoromethane sulfonate, CAS no: 34946-82-2; CuBr, CAS no: 7787-70-4; CuBr₂, CAS no: 7789-45-9; CuI, CAS no: 7681-65-4; CuI₂, CAS no 13767-71-0; Cu(NO₃)₂, CAS no: 3251-23-8; Cu(NO₃)₂×3 H₂O, CAS no: 10031-43-3; Cu(NO₃)₂×6 H₂O, CAS no: 13478-38-1; Cu(NO₃)₂×2,5 H₂O, CAS no: 19004-19-4; Cu(OH)₂, CAS no: 20427-59-2; Cu(ClO₄)₂×6 H₂O, CAS no: 10294-46-9; Cu(NH₃)₄SO₄×H₂O, CAS no: 10380-29-7; Cu(II)(OAc)₂, CAS no: 142-71-2; Cu(II)(OAc)₂, ×H₂O, CAS no: 6046-93-1; M_{I}(Cu(II)ₘXₙ)ₚ wherein M is an alkali metal comprising one of Li, K, Rb, Cs, or ammonium, Cu(II) is a divalent copper, X is a halogen atom selected from chlorine, bromine or iodine, 1 is an integer of 1 to 3, m is 1 or 2, n is an integer of 3 to 8, p is 1 or 2, and 1+2mp=np.

In a further embodiment, said copper catalyst exhibiting the oxidation state (+1) or (+2) is understood to be at least one compound of CuCl₂×2 H₂O, CAS no: 10125-13-0; CuCl₂, CAS no: 7447-39-4; CuCl, CAS no: 7758-89-6; CuCl₂×2 H₂O combined with LiCl, CAS no: 7447-39-4 or combined with LiCl×2H₂O, CAS no: 10125-13-0; CuCl₂×2 H₂O combined with MgCl₂, CAS no: 7786-30-3 or with MgCl₂×6 H₂O 7791-18-6; CuSO₄×5 H₂O, CAS no: 10257-54-2; Cu(II)trifluoromethane sulfonate, CAS no: 34946-82-2; CuBr, CAS no: 7787-70-4; CuBr₂, CAS no: 7789-45-9; CuI, CAS no: 7681-65-4; CuI₂, CAS no 13767-71-0; Cu(NO₃)₂, CAS no: 3251-23-8; Cu(NO₃)₂×3 H₂O, CAS no: 10031-43-3; Cu(NO₃)₂×6 H₂O, CAS no: 13478-38-1; Cu(NO₃)₂×2,5 H₂O, CAS no: 19004-19-4; Cu(OH)₂, CAS no: 20427-59-2; Cu(ClO₄)₂×6 H₂O, CAS no: 10294-46-9; Cu(NH₃)₄SO₄×H₂O, CAS no: 10380-29-7; Cu(II)(OAc)₂, CAS no: 142-71-2; Cu(II)(OAc)₂, ×H₂O, CAS no: 6046-93-1; M_{I}(Cu(II)ₘXₙ)ₚ wherein M is an alkali metal comprising one of Li, K, Rb, Cs, or ammonium, Cu(II) is a divalent copper, X is a halogen atom selected from chlorine, bromine or iodine, I is an integer of 1 to 3, m is 1 or 2, n is an integer of 3 to 8, p is 1 or 2, and I+2mp=np, being associated with at least one alkali metal halide or earth alkali metal halide.

In yet another embodiment, said copper catalyst exhibiting the oxidation state (+1) or (+2) is understood to be at least one compound of CuCl₂×2 H₂O, CAS no: 10125-13-0; CuCl₂, CAS no: 7447-39-4; Cu, CAS no: 7758-89-6; CuCl₂×2 H₂O combined with LiCl, CAS no: 7447-39-4 or combined with LiCl×2H₂O, CAS no: 10125-13-0; CuCl₂×2 H₂O combined with MgCl₂, CAS no: 7786-30-3 or with MgCl₂×6 H₂O 7791-18-6; CuSO₄×5 H₂O, CAS no: 10257-54-2; Cu(II)trifluoromethane sulfonate, CAS no: 34946-82-2; CuBr, CAS no: 7787-70-4; CuBr₂, CAS no: 7789-45-9; CuI, CAS no: 7681-65-4; CuI₂, CAS no 13767-71-0; Cu(NO₃)₂, CAS no: 3251-23-8; Cu(NO₃)₂×3 H₂O, CAS no: 10031-43-3; Cu(NO₃)₂×6 H₂O, CAS no: 13478-38-1; Cu(NO₃)₂×2,5 H₂O, CAS no: 19004-19-4; Cu(OH)₂, CAS no: 20427-59-2; Cu(ClO₄)₂×6 H₂O, CAS no: 10294-46-9; Cu(NH₃)₄SO₄×H₂O, CAS no: 10380-29-7; Cu(II)(OAc)₂, CAS no: 142-71-2; Cu(II)(OAc)₂, ×H₂O, CAS no: 6046-93-1; M_{I}(Cu(II)ₘXₙ)ₚ wherein M is an alkali metal comprising one of Li, K, Rb, Cs, or ammonium, Cu(II) is a divalent copper, X is a halogen atom selected from chlorine, bromine or iodine, I is an integer of 1 to 3, m is 1 or 2, n is an integer of 3 to 8, p is 1 or 2, and I+2mp=np, being associated with at least one alkali metal halide or earth alkali metal halide and with cupric hydroxide.

Said at least one alkali metal halide of the previous two embodiments is selected from the group consisting of NaCl, LiCl, KCl, CsCl, LiBr, NaBr, NH₄Br, KBr, CsBr, NaI, LiI, KI, CsI.

Said at least one earth alkali metal halide is selected form the group consisting of CaCl₂), CaBr₂, MgCl₂, MgBr₂.

In yet another embodiment, said copper catalyst exhibiting the oxidation state (+1) or (+2) is understood to be at least one compound of CuCl₂×2 H₂O, CAS no: 10125-13-0; CuCl₂, CAS no: 7447-39-4; CuCl, CAS no: 7758-89-6; CuCl₂×2 H₂O combined with LiCl, CAS no: 7447-39-4 or combined with LiCl×2H₂O, CAS no: 10125-13-0; CuCl₂×2 H₂O combined with MgCl₂, CAS no: 7786-30-3 or with MgCl₂×6 H₂O 7791-18-6; CuSO₄×5 H₂O, CAS no: 10257-54-2; Cu(II)trifluoromethane sulfonate, CAS no: 34946-82-2; CuBr, CAS no: 7787-70-4; CuBr₂, CAS no: 7789-45-9; CuI, CAS no: 7681-65-4; CuI₂, CAS no 13767-71-0; Cu(NO₃)₂, CAS no: 3251-23-8; Cu(NO₃)₂×3 H₂O, CAS no: 10031-43-3; Cu(NO₃)₂×6 H₂O, CAS no: 13478-38-1; Cu(NO₃)₂×2,5 H₂O, CAS no: 19004-19-4; Cu(OH)₂, CAS no: 20427-59-2; Cu(ClO₄)₂×6 H₂O, CAS no: 10294-46-9; Cu(NH₃)₄SO₄×H₂O, CAS no: 10380-29-7; Cu(II)(OAc)₂, CAS no: 142-71-2; Cu(II)(OAc)₂, ×H₂O, CAS no: 6046-93-1; M_{I}(Cu(II)ₘXₙ)ₚ wherein M is an alkali metal comprising one of Li, K, Rb, Cs, or ammonium, Cu(II) is a divalent copper, X is a halogen atom selected from chlorine, bromine or iodine, I is an integer of 1 to 3, m is 1 or 2, n is an integer of 3 to 8, p is 1 or 2, and I+2mp=np, being associated with at least one compound of a transition metal.

Said at least one compound of a transition metal is selected from the group consisting of Fe, Cr, Mn, Co, Ni, Zn, a halide of a rare earth metal like Ce, preferably from a halide of Fe, Cr, Mn, Co, Ni, Zn a rare earth metal like Ce and further preferred from a chloride of Fe, Cr, Mn, Co, Ni, Zn, a rare earth metal like Ce.

Typical representatives of M_{I}(Cu(II)ₘXₙ)ₚ as respectively indicated in the last seven sections are Li[CuCl₃]×2 H₂O, NH₄[CuCl₃]×2 H₂O, (NH₄)₂[CuCl₄]×2 H₂O), K[CuCl₃], K₂[CuCl₄]×2 H₂O, Cs[CuCl₃]×2 H₂O, Cs₂[CuCl₄]×2 H₂O, Cs₃[Cu₂Cl₇]×2 H₂O, Li₂[CuBr₄]×6 H₂O, K[CuBr₃], (NH₄)₂[CuBr₄]×2 H₂O, Cs₂[CuBr₄], and Cs[CuBr₃].

The experiments revealed that the copper catalyst as defined in at least one of the previous eight paragraphs could be reused without losing its catalytic activity. Thus, one cost-saving inventive embodiment reveals a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and said same copper catalyst being repeatedly or continuously employed.

The process of the invention aims to obtain from the chroman C1 the corresponding quinone C30 in high yield and purity. Thus, a further detailed process of the invention is the oxidation of at least one chroman C1 into a quinone C30, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2).

The quinone C30 is represented by the formula

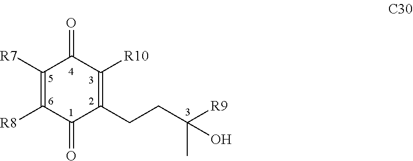

with R7, R8, R10 being H or CH₃; R9 being alkyl, alkenyl, and R9 preferably being alkyl of the formula C31.

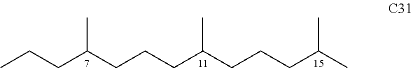 [SMILES: CCCC(C)CCCC(C)CCCC(C)C]

Alkyl with respect to R9 means C₁₀-C₂₀-alkyl, preferably C₁₄-C₁₈-alkyl and mostly preferred C₁₆-alkyl, viz entities comprising the given number of carbon atoms.

In one embodiment alkyl with respect to R9 is understood to have the structure of formula C31

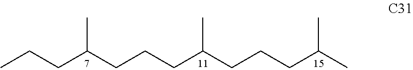 [SMILES: CCCC(C)CCCC(C)CCCC(C)C]

with the stereocenters in position 7, 11 having a 7R,11R-configuration, a 7R,11S configuration, a 7S,11R configuration or a 7S,11S configuration.

In one embodiment, the quinone C30 is α-tocopherol quinone of formula C32

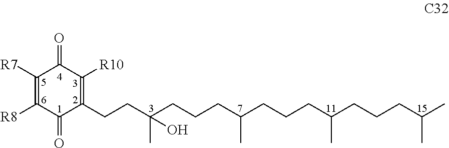

with R7, R8, R10, being CH₃, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is α-tocopherol quinone of formula C33

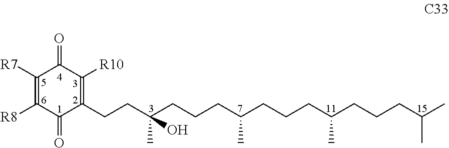

with R7, R8, R10, being CH₃, with the stereocenters of the lateral chain in position 3, 7, 11 having a 3R,7R,11R-configuration and the OH group in position 3 of the lateral chain having a 3R configuration.

Said preferred molecule is also named 2-[(3R,7R,11R)-3-hydroxy-3,7,11,15-tetramethylhexadecyl]-3,5,6-trimethyl-2,5-cyclohexadiene-1,4-dione, or 2-((7R,11R)-3-hydroxy-3,7,11,15-tetramethylhexadecyl)-3,5,6-trimethylcyclohexa-2,5-diene-1,4-dione, or (3R,7R,11R)-2-(3-hydroxy-3,7,11,15-tetramethylhexadec-1-yl)-3,5,6-trimethyl-1,4-benzoquinone, or 2-(3-hydroxy-3,7,11,15-tetramethylhexadecyl)-3,5,6-trimethyl-[1,4]benzoquinone, or (R,R,R)-α-tocopherol quinone, or para-α-tocopherylquinone, or d-α-tocopherolquinone.

In one embodiment, the quinone C30 is 3-tocopherol quinone of formula C34

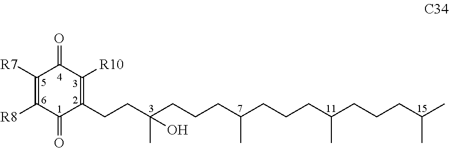

with R8, R10, being CH₃; R7 being H; with the stereocenters of the lateral chain in position 3,7,11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment, the quinone C30 is γ-tocopherol quinone of formula C35

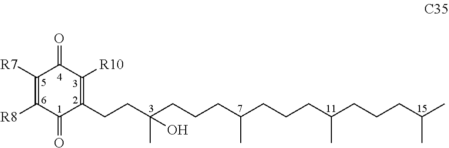

with R7, R8 being CH₃; R10 being H; with the stereocenters of the lateral chain in position 3,7,11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment, the quinone C30 is 5-tocopherol quinone of formula C36

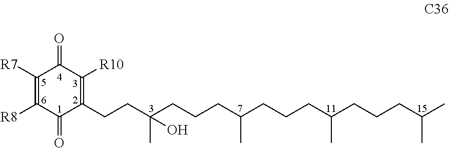

with R8 being CH₃; R7, R10 being H; with the stereocenters of the lateral chain in position 3,7,11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

Alkenyl with respect to R9 means C₁₀-C₂₀-alkenyl, preferably C₁₄-C₁₈-alkenyl and mostly preferred C₁₆-alkenyl, viz entities comprising the given number of carbon atoms and having at least one double bond.

In one embodiment alkenyl with respect to R9 is understood to have the structure of formula C37

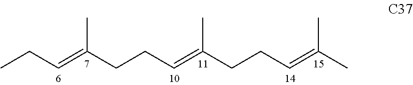 [SMILES: CC/C=C(\C)CC/C=C(\C)CCC=C(C)C]

with the methyl groups in position 7,11 having
a 7 cis,11 cis-conformation,
a 7 cis, 11 trans conformation,
a 7 trans,11 cis conformation or
a 7 trans, 11 trans conformation
   and the double bounds in position 6 and 10 having
a 6E,10E-configuration,
a 6E,10Z-configuration,
a 6Z,10E-configuration or
a 6Z,10Z-configuration.

In one embodiment alkenyl with respect to R9 is understood to have the structure of formula C38

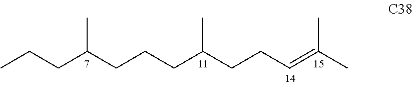 [SMILES: CCCC(C)CCCC(C)CCC=C(C)C]

with the stereocenters in position 7, 11 having a 7R,11R-configuration, a 7R,11S configuration, a 7S,11R configuration or a 7S,11S configuration and the double bond in position 14.

In one embodiment alkenyl with respect to R9 is understood to have the structure of formula C39

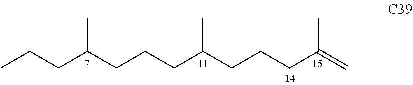 [SMILES: C=C(C)CCCC(C)CCCC(C)CCC]

with the stereocenters in position 7, 11 having a 7R,11R-configuration, a 7R,11S configuration, a 7S,11R configuration or a 7S,11S configuration.

In one embodiment the quinone C30 is α-tocotrienol quinone of formula C40

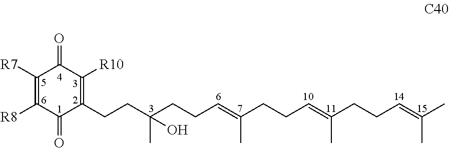

with R7, R8, R10 being CH₃,
with the stereocenter of the lateral chain in position 3 having a 3R configuration,
the methyl groups in position 7,11 having a cis conformation and
the double bonds in positions 6,10 having an E-configuration;
   with R7, R8, R10 being CH₃,
with the stereocenter of the lateral chain in position 3 having a 3R configuration,
the methyl group in position 7 having a cis conformation,
the methyl group in position 11 having a trans conformation,
the double bond in position 6 having an E-configuration and
the double bond in position 10 having a Z-configuration;
   with R7, R8, R10 being CH₃,
with the stereocenter of the lateral chain in position 3 having a 3R configuration,
the methyl group in position 7 having a trans conformation,
the methyl group in position 11 having a cis conformation,
the double bond in position 6 having a Z-configuration and
the double bond in position 10 having an E-configuration;
   with R7, R8, R10 being CH₃,
with the stereocenter of the lateral chain in position 3 having a 3R configuration,
the methyl groups in position 7,11 having a trans conformation and,
the double bonds in positions 6,10 having a Z-configuration;
   with R7, R8, R10 being CH₃,
with the stereocenter of the lateral chain in position 3 having a 3S configuration,
the methyl groups in position 7,11 having a cis conformation and
the double bonds in positions 6,10 having an E-configuration;
   with R7, R8, R10 being CH₃,
with the stereocenter of the lateral chain in position 3 having a 3S configuration,
the methyl group in position 7 having a cis conformation,
the methyl group in position 11 having a trans conformation,
the double bond in position 6 having an E-configuration and
the double bond in position 10 having a Z-configuration;
   with R7, R8, R10 being CH₃,
with the stereocenter of the lateral chain in position 3 having a 3S configuration,
the methyl group in position 7 having a trans conformation,
the methyl group in position 11 having a cis conformation,
the double bond in position 6 having a Z-configuration and
the double bond in position 10 having an E-configuration;
   with R7, R8, R10 being CH₃,
with the stereocenter of the lateral chain in position 3 having a 3S configuration,
the methyl groups in position 7,11 having a trans conformation and
the double bonds in positions 6,10 having a Z-configuration.

In one embodiment the quinone C30 is α-tocotrienol quinone of formula C41

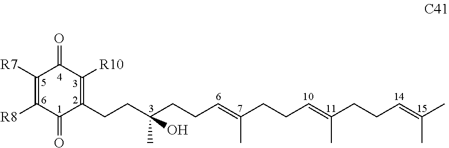

with R7, R8, R10 being CH₃,
with the stereocenter of the lateral chain in position 3 having a 3R configuration,
the methyl groups in position 7,11 having a cis conformation,
the double bonds in positions 6,10 having an E-configuration and
the OH group in position 3 of the lateral chain having a 3R configuration.

In one embodiment the quinone C30 is β-tocotrienol quinone of formula C42

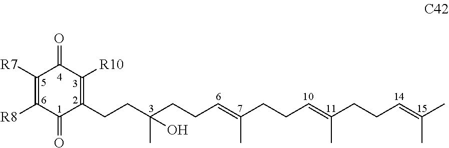

with R8, R10 being CH₃, R7 being H,
with the stereocenter of the lateral chain in position 3 having a 3R configuration,
the methyl groups in position 7,11 having a cis conformation and
the double bonds in positions 6,10 having an E configuration,
   with R8, R10 being CH₃, R7 being H,
with the stereocenter of the lateral chain in position 3 having a 3R configuration,
the methyl group in position 7 having a cis conformation,
the methyl group in position 11 having a trans conformation;
the double bond in position 6 having an E-configuration and
the double bond in position 10 having a Z-configuration;
   with R8, R10 being CH₃, R7 being H,
with the sterreocenter of the lateral chain in position 3 having a 3R configuration,
the methyl group in position 7 having a trans conformation,
the methyl group in position 11 having a cis conformation,
the double bond in position 6 having a Z-configuration and
the double bond in position 10 having a E-configuration;
   with R8, R10 being CH₃, R7 being H,
with the stereocenter of the lateral chain in position 3 having a 3R configuration,
the methyl groups in position 7,11 having a trans conformation and
the double bonds in positions 6,10 having a Z-configuration;
   with R8, R10 being CH₃, R7 being H,
with the stereocenter of the lateral chain in position 3 having a 3S configuration,
the methyl groups in position 7,11 having a cis conformation and
the double bonds in positions 6,10 having an E-configuration;
   with R8, R10 being CH₃, R7 being H,
with the stereocenter of the lateral chain in position 3 having a 3S configuration,
the methyl group in position 7 having a cis conformation,
the methyl group in position 11 having a trans conformation,
the double bond in position 6 having an E-configuration and
the double bond in position 10 having a Z-configuration;
   with R8, R10 being CH₃, R7 being H,
with the stereocenter of the lateral chain in position 3 having a 3S configuration,
the methyl group in position 7 having a trans conformation,
the methyl group in position 11 having a cis conformation,
the double bond in position 6 having a Z-configuration and
the double bond in position 10 having an E-configuration;
   with R8, R10 being CH₃, R7 being H,
with the stereocenter of the lateral chain in position 3 having a 3S configuration,
the methyl groups in position 7,11 having a trans conformation and
the double bonds in positions 6,10 having a Z-configuration.

In one embodiment the quinone C30 is γ-tocotrienol quinone of formula C43

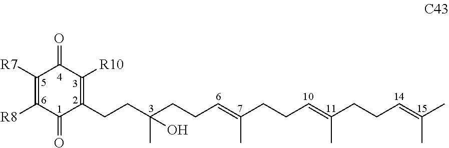

with R7, R8 being CH₃, R10 being H,
with the stereocenter of the lateral chain in position 3 having a 3R configuration,
the methyl groups in position 7,11 having a cis conformation and
the double bonds in positions 6,10 having an E-configuration;
   with R7, R8 being CH₃, R10 being H,
with the stereocenter of the lateral chain in position 3 having a 3R configuration,
the methyl group in position 7 having a cis conformation,
the methyl group in position 11 having a trans conformation,
the double bond in position 6 having an E-configuration and
the double bond in position 10 having a Z-configuration;
   with R7, R8 being CH₃, R10 being H,
with the stereocenter of the lateral chain in position 3 having a 3R configuration,
the methyl group in position 7 having a trans conformation,
the methyl group in position 11 having a cis conformation,
the double bond in position 6 having a Z-configuration and
the double bond in position 10 having an E-configuration;
   with R7, R8 being CH₃, R10 being H,
with the stereocenter of the lateral chain in position 3 having a 3R configuration,
the methyl groups in position 7,11 having a trans conformation and
the double bonds in positions 6,10 having a Z-configuration;
   with R7, R8 being CH₃, R10 being H,
with the stereocenter of the lateral chain in position 3 having a 3S configuration
the methyl groups in position 7,11 having a cis conformation and
the doubles bond in positions 6,10 having an E-configuration;
   with R7, R8 being CH₃, R10 being H,
with the stereocenter of the lateral chain in position 3 having a 3S configuration,
the methyl group in position 7 having a cis conformation,
the methyl group in position 11 having a trans conformation,
the double bond in position 6 having an E-configuration and
the double bond in position 10 having a Z-configuration;
   with R7, R8 being CH₃, R10 being H,
with the stereocenter of the lateral chain in position 3 having a 3S configuration,
the methyl group in position 7 having a trans conformation,
the methyl group in position 11 having a cis conformation,
the double bond in position 6 having a Z-configuration and
the double bond in position 10 having a E-configuration;
   with R7, R8 being CH₃, R10 being H,
with the stereocenter of the lateral chain in position 3 having a 3S configuration,
the methyl groups in position 7,11 having a trans conformation and
the double bonds in positions 6,10 having a Z-configuration.

In one embodiment the quinone C30 is 5-tocotrienol quinone of formula C44

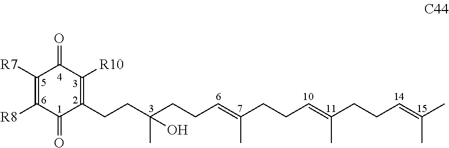

with R8 being CH₃, R7, R10 being H,
with the stereocenter of the lateral chain in position 3 having a 3R configuration,
the methyl groups in position 7,11 having a cis conformation and
the double bonds in positions 6,10 having an E-configuration;
   with R8 being CH₃, R7, R10 being H,
with the stereocenter of the lateral chain in position 3 having a 3R configuration,
the methyl group in position 7 having a cis conformation,
the methyl group in position 11 having a trans conformation,
the double bond in position 6 having an E-configuration and
the double bond in position 10 having a Z-configuration;
   with R8 being CH₃, R7, R10 being H,
with the stereocenter of the lateral chain in position 3 having a 3R configuration,
the methyl group in position 7 having a trans conformation,
the methyl group in position 11 having a cis conformation,
the double bond in position 6 having a Z-configuration and
the double bond in position 10 having an E-configuration;
   with R8 being CH₃, R7, R10 being H,
with the stereocenter of the lateral chain in position 3 having a 3R configuration,
the methyl groups in position 7,11 having a trans conformation and
the double bonds in positions 6,10 having a Z-configuration;
   with R8 being CH₃, R7, R10 being H,
with the stereocenter of the lateral chain in position 3 having a 3S configuration,
the methyl groups in position 7,11 having a cis conformation and
the double bonds in positions 6,10 having an E-configuration;
   with R8 being CH₃, R7, R10 being H,
with the stereocenter of the lateral chain in position 3 having a 3S configuration,
the methyl group in position 7 having a cis conformation,
the methyl group in position 11 having a trans conformation,
the double bond in position 6 having an E-configuration and
the double bond in position 10 having a Z-configuration;
   with R8 being CH₃, R7, R10 being H,
with the stereocenter of the lateral chain in position 3 having a 3S configuration,
the methyl group in position 7 having a trans conformation,
the methyl group in position 11 having a cis conformation
the double bond in position 6 having a Z-configuration and
the double bond in position 10 having an E-configuration;
   with R8 being CH₃, R7, R10 being H,
with the stereocenter of the lateral chain in position 3 having a 3S configuration,
the methyl groups in position 7, 11 having a trans conformation and
the double bonds in positions 6,10 having a Z-configuration.

In one embodiment the quinone C30 is α-tocomonoenol quinone of formula C45

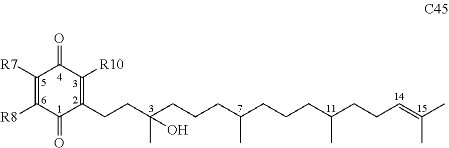

with R7, R8, R10, being CH₃, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is α-tocomonoenol quinone of formula C46

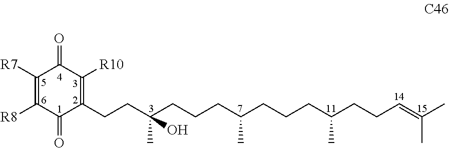

with R7, R8, R10, being CH₃, with the stereocenters of the lateral chain in position 3, 7, 11 having a 3R,7R,11R-configuration and the OH group in position 3 of the lateral chain having a 3R configuration.

In one embodiment the quinone C30 is β-tocomonoenol quinone of formula C47

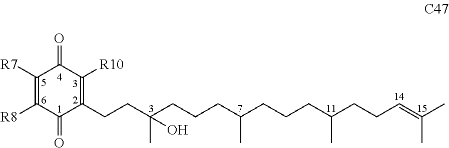

with R7 being H, R8, R10, being CH₃, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is γ-tocomonoenol quinone of formula C48

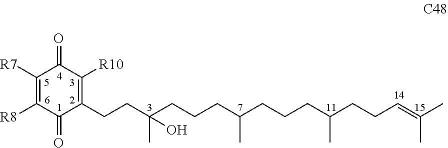

with R7, R8 being CH₃, R10 being H, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is δ-tocomonoenol quinone of formula C49

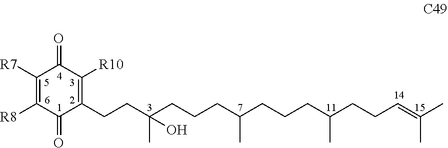

with R7, R10 being H, R8 being CH₃, with the stereocenters of the lateral chain in position 3,7,11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is a quinone of a marine-derived α-tocopherol (α-MDT) of formula C50

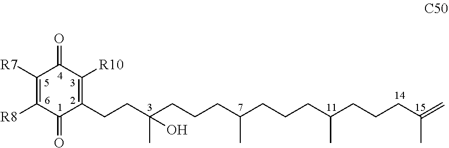

with R7, R8, R10, being CH₃, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is a quinone of a marine-derived α-tocopherol (α-MDT) of formula C51

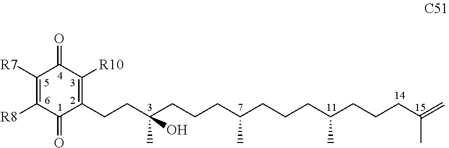

with R7, R8, R10, being CH₃, with the stereocenters of the lateral chain in position 3, 7, 11 having a 3R,7R,11R-configuration and the OH group in position 3 of the lateral chain having a 3R configuration.

In one embodiment the quinone C30 is a quinone of a marine-derived β-tocopherol (β-MDT) of formula C52

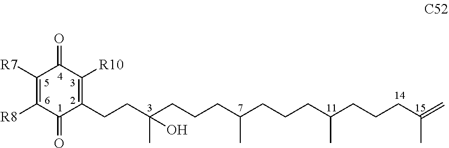

with R7 being H, R8, R10, being CH₃, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is a quinone of a marine-derived γ-tocopherol (γ-MDT) of formula C53

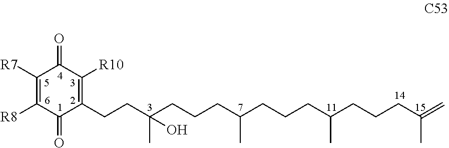

with R7, R8 being CH₃, R10 being H, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is a quinone of a marine-derived 5-tocopherol (5-MDT) of formula C54

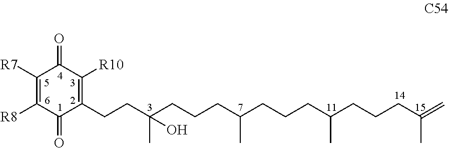

with R7, R10 being H, R8 being CH₃, with the stereocenters of the lateral chain in position 3, 7, 11 having a 3R,7R,11R-configuration; a 3R,7R,11S configuration; a 3R,7S,11R configuration; a 3S,7R,11R configuration; a 3R,7S,11S configuration; a 3S,7R,11S configuration; a 3S,7S,11R configuration or a 3S,7S,11S configuration, and the OH group in position 3 of the lateral chain having a 3R or 3S configuration.

In one embodiment the quinone C30 is a mixture of at least two of the embodiments C32, C33, C34, C35, C36, C40, C41, C42, C43, C44, C45, C46, C47, C48, C49, C50, C51, C52, C53, C54.

As can be seen from the examples and the comparative examples, it is beneficial for obtaining high yields of quinone C30 of the invention in reasonable reaction times, if the gaseous compound is not only in contact with the surface of the reaction mixture, but with the entire part of said reaction mixture. This can be achieved by shaking the reaction mixture under a gaseous atmosphere containing oxygen. However, prominent results are achieved, if the gaseous compound travels through the reaction mixture. Therefore, an embodiment of the invention seeks protection for the gaseous compound comprising, essentially consisting of, or consisting of oxygen being actively moved through the solvent mixture comprising at least two solvents or through the C-bearing solvent. Actively moving means applying a gas or a gaseous compound by a pressure means to the reaction mixture with a pressure being higher than ambient pressure. Such motion makes sure that the gas or the gaseous compound continuously enters in excess into the reaction mixture and the not reacted part thereof afterwards leaves the reaction vessel. Actively moving also means applying a gas or a gaseous compound by a pressure means to the reaction mixture with a pressure being higher than ambient pressure, the application being such that the gas being liberated under the surface of the solvent mixture comprising at least two solvents or through the C-bearing solvent.

One embodiment of the invention uses the so-called off-gas or exhaust gas mode, meaning that the gaseous compound continuously travelling through the reaction mixture leaves it without any further use. This mode is advantageously used with less expensive gaseous compounds like air, making the synthetic installation or plant less complex and less expensive. This embodiment is defined by a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), with the gaseous compound being actively moved in an off-gas mode through the solvent mixture comprising at least two solvents or through the C-bearing solvent.

A different embodiment of the invention uses the so-called circle-gas mode, which is defined by injecting the gaseous compound into the reaction means, collecting excess gaseous compound at a different point of the reaction means, supplementing said collected excess gaseous compound depleted in oxygen or oxygen-containing compound with fresh oxygen or oxygen-containing compound and reintroducing the thus recycled gaseous compound into the reaction means. This embodiment is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), with the gaseous compound being actively moved in a circle-gas mode through the solvent mixture comprising at least two solvents or through the C-bearing solvent.

A crucial topic of the invention is to use an appropriate amount of catalyst, especially copper catalyst. This is in order to increase yield, to reduce reaction costs and to mostly minimize the amount of side products or of traces of raw materials with respect to the respective catalyst employed. Thus an important characteristic of the invention determines the copper catalyst being used in an amount ranging from 0,001 to 10 molar equivalents with respect to the molar amount of chroman C1 used, preferably in a stoichiometric or almost stoichiometric amount, even more preferably in a substoichiometric amount, further preferred in an amount ranging from 0,01 to 0,95 molar equivalents, yet further preferred in an amount ranging from 0,01 to 0,75 molar equivalents, still further preferred in an amount ranging from 0,1 to 0,5 molar equivalents, further preferred in an amount ranging from 0,1 to 0,35 molar equivalents and most preferably in an amount ranging from 0,11 to 0,25 molar equivalents. For instance, examples, 968 (CN10) 952 (CN11), 985 (CN12), 988 (CN13), 905 (CN14), 1052 (CN15), 1086 (CN16), 977 (CN17) and 979 (CN18) reveal stochiometric and sub-stoichiometric amounts of at least one catalyst used to give higher yields of quinone C30 under the claimed conditions (cf. in relation thereto comparative examples 1004 (CN7), 903 (CN8) referring to WO 2011 139897 A2, which do not use oxygen or actively introduce a gaseous compound).

Upon comparing the different types of copper catalysts, the copper halides were shown to give high yields in short reaction times (cf. examples 977 (CN19),1052 (CN20), 1021 (CN21), 1060 (CN22), 946 (CN23), 1054 (CN24), 1032 (CN25), 877 (CN26), 905 (CN27), 935 (CN28), 942 (CN29), 952 (CN11), 976 (CN31)). Reaction time within this disclosure is meant to be the total reaction time, viz. the time for adding the chroman C1, plus the time for adding the gaseous compound plus, if given, the time for further stirring. Thus, one embodiment of the invention determines the copper catalyst to be a copper halide, preferably a copper chloride and mostly preferred CuCl₂.

This is also reflected by the important embodiment of the invention disclosing a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst being a copper halide exhibiting the oxidation state (+1) or (+2) and said process being realized in a time ranging from 2 h to 23 h, preferably from 2,6 h to 15 h more preferably from 3 h to 10 h, further preferred from 3 h to 9 h, still further preferred from 3 h to 7 h, further preferred from 3 h to 6,3 h still further preferably from 3,6 h to 6 h, and most preferably 4 h to 5 h including 4,75 and 4,8 h.

Most pertinent results with respect to reaction time and yield were obtained, when the copper catalyst used is CuCl₂(cf. examples 1021 (CN21), 1032 (CN25), 1060 (CN22), 877 (CN26), 905 (CN27)). Therefore the previous embodiment is further developed such that it discloses a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst being selected from the group consisting of CuCl₂×2 H₂O, CAS no: 10125-13-0; CuCl₂, CAS no: 7447-39-4; and said process being realized in a time ranging from 2 h to 23 h, preferably from 2,6 h to 15 h more preferably from 3 h to 10 h, further preferred from 3 h to 9 h, still further preferred from 3 h to 7 h, further preferred from 3 h to 6,3 h, yet more preferably from 3,6 h to 6 h and most preferably from 4 h to 5 h including 4,75 and 4,8 h.

Satisfactory results with respect to yield, reaction time and reaction temperature were also obtained, when the copper catalyst is used together with a further metal compound (cf. exam pies 941(CN32), 946 (CN33). Accordingly a further embodiment of the invention is a process wherein the copper catalyst is combined with at least one metal compound selected form the group consisting of Na, Li, K, Cs, Mg, Ca, Sr, Ba, Fe, Cr, Mn, Co, Ni, Zn, La, Ce, Pr, Nd compounds, preferably with one metal halide of the aforementioned group, more preferred with at least one metal chloride of said group and mostly preferred with LiCl and/or MgCl₂.

For the embodiments of the invention using at least one metal compound in addition to the copper catalyst, the amount of metal compound used with respect to the chroman C1 has an impact on the formation of quinone C30. High yields of quinone C30 were obtained (cf. examples 941 (CN32), 946 (CN35), 390 (CN34), 952 (CN30)), when the process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and being combined with at least one metal compound selected form the group consisting of Na, Li, K, Cs, Mg, Ca, Sr, Ba, Fe, Cr, Mn, Co, Ni, Zn, La, Ce, Pr, Nd compounds, defines the molar ratio between said at least one metal compound and the at least one chroman C1 ranging from 0,1 to 10, preferably from 0,2 to 5 and mostly preferred from 0,4 to 1 including 0,5.

This observation in particular holds for the embodiment where the process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and being combined with at least one metal halide selected form the group consisting of Na, Li, K, Cs, Mg, Ca, Sr, Ba, Fe, Cr, Mn, Co, Ni, Zn, La, Ce, Pr, Nd compounds, defines the molar ratio between said at least one metal compound and the at least one chroman C1 ranging from 0,1 to 10, preferably from 0,2 to 5 and mostly preferred from 0,4 to 1 including 0,5.

Even more persuasive yields were obtained with the process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and being combined with at least one metal compound selected form the group consisting of LiCl and/or MgCl₂, defines the molar ratio between said at least one metal compound and the at least one chroman C1 ranging from 0,1 to 10, preferably from 0,2 to 5 and mostly preferred from 0,4 to 1 including 0,5.

In one embodiment of the invention the way of obtaining the reaction mixture (comprising chroman C1, the solvent mixture or the C-bearing solvent, the gaseous compound the copper catalyst and the further metal compound) is straight forward, since it does not require any additional effort to get the copper catalyst and optionally the further metal compound solubilized or finely dispersed in said reaction mixture. This embodiment is any one of the claimed or disclosed embodiments wherein the copper catalyst and optionally the at least one metal compound are added to the solvent mixture or to the C-bearing solvent in form of an aqueous solution. Thus, particularly preferred is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), wherein an aqueous solution of the copper catalyst and optionally of the at least one metal compound are added to the solvent mixture or to the C-bearing solvent.

Provided the solvent mixture comprising at least two solvents comprises a hydrophilic solvent, preferably water, another feature makes the inventive process fast and thus cost-effective. This feature defines for every claimed or disclosed embodiment of the invention the copper catalyst and optionally the at least one metal compound being solubilized in the aqueous phase of the solvent mixture comprising at least two solvents. This feature is especially favorable, if the inventive process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), determines the copper catalyst and optionally the at least one metal compound being solubilized in the aqueous phase of the solvent mixture comprising at least two solvents.

Both previously mentioned embodiments serve for a quick solubilization of the reagents required and omit the tedious synthesis of a complexed catalyst.

According to the experiments realized, it was found beneficial to have a certain concentration of the copper catalyst in one of the at least two solvents of the solvent mixture (cf. examples 960 (CN37), 974 (CN38), 958 (CN39), 952 (CN40), 971 (CN41)). Beneath and above said concentration of the copper catalyst, yields of quinone C30 were smaller and/or reaction time was higher. However, provided one respects for each of the claimed or disclosed embodiments the concentration of the copper catalyst to range from 5 to 70 w % based on one solvent of the solvent mixture comprising at least two solvents or based on the C-bearing solvent, considerable yields of quinone C30 in reasonable reaction times are obtained, especially if the copper catalyst is selected from CuCl₂×2 H₂O, CAS no: 10125-13-0 or CuCl₂, CAS no: 7447-39-4. This in particular holds for a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and its concentration ranging from 5 to 70 w % based on one solvent of the solvent mixture comprising at least two solvents or based on the C-bearing solvent, especially if the copper catalyst is selected from CuCl₂×2 H₂O, CAS no: 10125-13-0 or CuCl₂, CAS no: 7447-39-4. Even improved yields can be obtained if the concentration of the copper catalyst ranges from 10 to 50 w % based on one solvent of the solvent mixture comprising at least two solvents or based on the C-bearing solvent.

As already mentioned supra, some embodiments of the invention use the copper catalyst in combination with at least one metal compound. High yields of quinone C30 in a reasonable reaction time were obtained, if for each of the at least one metal compound containing claimed or disclosed embodiments, the concentration of the at least one metal compound in one solvent of the solvent mixture comprising at least two solvents or in the C-bearing solvent ranges from 5 to 80 w %. A further embodiment of the invention thus seeks protection for a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), and being combined with at least one metal compound selected form the group consisting of Na, Li, K, Cs, Mg, Ca, Sr, Ba, Fe, Cr, Mn, Co, Ni, Zn, La, Ce, Pr, Nd compounds, preferably with one metal halide of the aforementioned group, more preferred with at least one metal chloride of said group and mostly preferred with LiCl and/or MgCl₂, wherein the concentration of the at least one metal compound in one solvent of the solvent mixture comprising at least two solvents or in the C-bearing solvent ranges from 5 to 80 w %.

The inventive process was shown to be suitable for various chromans. It was not only successfully realized with tocopherols but also with tocotrienols in particular with α-tocopherol or α-tocotrienol. A further important embodiment of the invention thus discloses the inventive process wherein the chroman C1 is α-tocopherol of formula C3, C4, C5 or α-tocotrienol of formula C12, C13, C14. This is to say the chroman C1 used in the inventive process is at least one of the group consisting of α-tocopherol of formula C3, C4, C5 and α-tocotrienol of formula C12, C13, C14.

Further trials were undertaken in order to determine the appropriate amount of chroman C1 in the reaction mixture of the inventive process. 5 to 80 w %, preferably 20 to 50 w % of chroman C1 based on one solvent of the solvent mixture comprising at least two solvents or based on the C-bearing solvent were shown for each of the claimed or disclosed embodiments to give high yields in short reaction times (cf. examples 872 (CN42), 1052 (CN1) in comparison to 875 (CN44)). This in particular is reflected by a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) wherein the amount of chroman C1 used ranges from 5 to 80 w %, preferably from 20 and 50 w % based on one solvent of the solvent mixture comprising at least two solvents or based on the C-bearing solvent.

The inventive process is suited to be realized either batchwise or semi-batchwise, with batchwise meaning the chroman C1, the gaseous compound and the copper catalyst being reacted in the solvent mixture or in the C-bearing solvent, the obtained reaction mixture being subjected to a work-up and the inventive process being started again with a new set of starting compounds. Semi-batchwise is understood to conduct the inventive process such that, some of the reagents like e.g. the gaseous compound are continuously added to the reaction mixture, whereas some other reagents like e.g. the chroman C1 are added, reacted, the reaction product removed, and new reagent C1 is again added. Likewise, semi-batchwise is understood to conduct the inventive process such that the catalyst and the solvent mixture or the C-bearing solvent are charged into the reactor, the chroman C1, optionally solved in one of the solvents, is added to the catalyst or solvent mixture over a certain period of time followed by stirring until full conversion, while the gaseous compound is continuously added over a period starting from the addition of chroman C1 and until full conversion, the obtained reaction mixture being subjected to a work-up and the inventive process being started again with a new set of starting compounds.

One further embodiment defines a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) said process being realized batchwise.

Still another embodiment defines a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) said process being realized semi-batchwise.

A further aspect of the invention is the simplicity of the process. It can be realized with the raw material chroman C1, the gaseous compound and the copper catalyst altogether in the solvent mixture or in the C-bearing solvent. Any auxiliary reagents like detergents, emulsifiers, wetting agents, phase transfer reagents or the like are not required at all. This makes any purification steps at the end of the inventive process straight forward and time-saving. The embodiment disclosing the solvent mixture comprising at least two solvents or the C-bearing solvent being free of any detergent thus is very important to the invention.

The chroman C1 readily dissolves in a lipophilic solvent whereas the copper catalyst can be easily solubilized in water. This is advantageous, since without mixing, lipophilic solvent and water in many cases separate, thus also separating the respectively solubilized reagents. With other words, the inventive process realized in a mixture of a lipophilic solvent and water will be stopped immediately upon interrupting the stirring means. This provides the skilled person with the possibility to easily control reaction progress and reaction time. Furthermore, copper catalyst or copper catalyst and at least one metal compound on one hand and chroman C1 and/or quinone C30 on the other hand separate immediately without any additional step or procedural burden.

This favorable feature is reflected by the following two embodiments viz:

A process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) with the solvent mixture comprising at least two solvents being intensively stirred. Intensively stirred within this disclosure means 600 to 1500 revolutions per minute (rpm), preferably 700 to 1200 revolutions per minute (rpm) and mostly preferred 1000 to 1200 revolutions per minute (rpm).

A process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) with the at least two solvents of the solvent mixture comprising water and an organic solvent, preferably an organic solvent which is not miscible with water.

One solvent of the solvent mixture comprising at least two solvents or the organic solvent is selected from the group consisting of alcohols, diols, aliphatic hydrocarbons, aromatic hydrocarbons, ethers, glycol ethers, polyethers, polyethylene glycol, ketones, esters amides, nitriles, halogenated solvents, carbonates, dimethyl sulfoxide and sulfolane.

Said one solvent or the organic solvent in one embodiment almost does not mix with water, preferably does not mix at all with water.

The term alcohol within this invention comprises at least one primary, secondary or tertiary alcohol having from 1 to 18 carbon atoms, preferably at least one saturated primary, secondary or tertiary alcohol having from 1 to 18 carbon atoms.

Said at least one, preferably saturated, primary, secondary or tertiary alcohol having from 1 to 18 carbon atoms is selected from the group consisting of methanol, ethanol, propanol, isopropanol, 1-butanol, 2-butanol, 2-methyl-1-propanol, tert-butyl alcohol, pentanol in all its isomeric forms, for example 1-pentanol or n-pentanol or n-amyl alcohol, 3-methylbutan-1-ol or isoamyl alcohol, 2-methyl-1-butanol, 2.2-dimethylpropan-1-ol, 2-pentanol, 3-pentanol, 3-methyl-2-butanol, 2-methyl-2-butanol, cyclopentanol, hexanol in all its isomeric forms, for example 1-hexanol or n-hexanol, cyclohexanol, 2-methyl-1-pentanol, 3-methyl-1-pentanol, 4-methyl-1-pentanol, 2,2-dimethyl-1-butanol, 1,3-dimethyl-1-butanol, 2,3-dimethyl-1-butanol, 3,3-dimethyl-1-butanol, 2-ethylbutan-1-ol, 2-hexanol, 3-hexanol, 3-methyl-2-pentanol, 4-methyl-2-pentanol, 3,3-dimethyl-2-butanol, 2-methyl-2-pentanol, 3-methylpentan-3-ol, 2,3-dimethyl-2-butanol, methyylcyclopentanol, heptanol in all its isomeric forms, for example 1-heptanol, 2-heptanol, 3-heptanol, 3-ethyl-3-pentanol, octanol in all its isomeric forms, for example 1-octanol, 2-octanol, 2-ethylhexanol, 1-nonanol, 1-decanol, 2-ethyl-1-hexanol, 1-dodecanol, 1-tetradecanol, 1-hexadecanol, 1-octadecanol.

Higher primary, secondary or tertiary alcohols were shown to be less sensitive towards ignition, which is preferred for the inventive process working under or with a gaseous compound comprising or consisting of oxygen. In addition, they scarcely or not at all mix with water, thus they can be easily separated from an aqueous fraction.

In a preferred embodiment of the invention alcohol therefore is understood to be at least one primary, secondary or tertiary alcohol having from 5 to 18 carbon atoms, preferably at least one saturated primary, secondary or tertiary alcohol having from 5 to 18 carbon atoms.

In another preferred embodiment of the invention alcohol is understood to be at least one primary, secondary or tertiary alcohol having from 6 to 18 carbon atoms, preferably at least one saturated primary, secondary or tertiary alcohol having from 6 to 18 carbon atoms.

For its ease of availability, alcohol is at least one primary, secondary or tertiary alcohol having from 5 to 8 carbon atoms, preferably at least one saturated primary, secondary or tertiary alcohol having from 5 to 8 carbon atoms.

Availability revealed said alcohol being at least one, preferably saturated, primary, secondary or tertiary alcohol being selected from the group consisting of 1-pentanol, 1-hexanol or n-hexanol, 2-ethylhexanol, 3-heptanol, 2-octanol, 3-ethyl-3-pentanol, 1.3-dimethyl butanol or amylmethyl alcohol, diacetone alcohol, methylisobutyl carbinol or 4-methyl-2-pentanol, tert.-hexyl alcohol, cyclohexanol, 1,6-hexanediol, 1,5 hexanediol, 1,4-hexanediol, 1,3-hexanediol, 2-methyl-2,4-pentanediol, pinacol or 2,3-dimethyl-2,3-butanediol, 1,2,5-hexanetriol, 1,2,6-hexanetriol, trimethylolpropane.

Another aspect of the inventive process focuses on low amounts of inventive process reagents or components thereof to be associated with the quinones formed. This can be promoted or achieved with a special type of alcohol used. In a preferred embodiment of the invention alcohol therefore is understood to be at least one secondary or tertiary alcohol having from 5 to 18 carbon atoms, preferably at least one saturated secondary or tertiary alcohol having from 5 to 18 carbon atoms.

A valuable embodiment of the invention thus is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) with the solvent mixture comprising at least two solvents being a mixture of water and as organic solvent at least one primary, secondary or tertiary alcohol having from 6 to 18 carbon atoms, preferably at least one saturated secondary or tertiary alcohol having from 6 to 18 carbon atoms.

A further elaborated valuable embodiment of the invention thus is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) with the solvent mixture comprising at least two solvents being a mixture of water and at least one secondary or tertiary alcohol having from 5 to 18 carbon atoms, preferably at least one saturated secondary or tertiary alcohol having from 5 to 18 carbon atoms.

Diol of this disclosure is understood to be at least one compound selected from the group consisting of 1,2-ethanediol or ethylene glycol, 1,2-propanediol, 1,3-propanediol, 1,2-butanediol, 2,3.butanediol, 1,3-butanediol, 2-methyl-1,2-prropanediol, 1,4-butanediol, 1,2-pentanediol, 1,3-pentanediol, 1,4-pentanediol, 1,5-pentanediol, 2,2-dimethyl-3-propanediol, 3-methyl-2,4-pentanediol, 4-hydroxy-4-methyl-2-pentanol, 1,6-hexane diol, 1,5 hexane diol, 1,4-hexane diol, 1,3-hexane diol, 2-methyl-2,4-pentane diol, pinacol, 2,3-dimethyl-2,3-butane diol, diethylene glycol, triethylene glycol, glycerol, 1,2-butylene glycol, 1,2,3-butanetriol, 1,2,4-butanetriol, 2-methyl-2,3-butanediol.

Aliphatic hydrocarbon of this disclosure is understood to be selected from the group consisting of n-pentane, iso-pentane, neo-pentane, n-hexane, hexane in all its isomeric forms, n-heptane, heptane in all its isomeric forms, cyclopentane, cyclohexane, cycloheptane, methyl cyclohexane, octane in all its isomeric forms, nonane in all its isomeric forms, decane in all its isomeric forms, undecane in all its isomeric forms, dodecane in all its isomeric forms polyethylene and nitromethane.

Aromatic hydrocarbon within the content of this disclosure is understood to be selected form the group consisting of benzene, toluene, xylene in all its isomeric forms e.g. o-, m- or p-xylene, ethylbenzene, 1,3,5-trimethylbeneze, isopropylbenzen, diisopropylbeneze in all its isomeric forms, 2-isopropyltoluene, 3-isopropyltoluene, 4-isopropyltoluene and nitrobenzene.

Ether within the content of this disclosure is understood to be selected form the group consisting of dimethyl ether, diethyl ether, di-n-propyl ether, diisopropyl ether, methyl ethyl ether, dibutyl ether, dipentyl ether, diisopentyl ether, n-butyl methyl ether, sec-butyl methyl ether, tert-butyl methyl ether, tert-butyl ethyl ether, methyl isobutyl ether, tetrahydrofuran, 2-methyltetrahydrofuran, 3-methyltetrahydrofuran, 2,5-Dimethyltetrahydrofuran, 1,3-dioxolane, tetrahydropyran, 1,4-dioxane, 1,3,5-trioxane, benzylethylether, cyclopentyl methyl ether and anisole.

Glycol ether or polyether within the content of this disclosure is understood to be selected form the group consisting of dimethoxymethane, diethoxymethane, ethylene glycol monomethyl ether, ethylene glycol dimethyl ether, ethylene glycol monoethyl ether, ethylene glycol diethyl ether, ethylene glycol monoisopropyl ether, dipropylene glycole, ethylene glycol monobutyl ether, ethylene glycol dibutyl ether, diethylene glycol monomethyl ether, diethylene glycol dimethyl ether, diethylene glycol monoethyl ether, diethylene gylcol diethyl ether, diethylene glycol diacetate, triethylene glycol dimethyl ether, triethylene glycol diethyl ether, tetramethylene glycol dimethyl ether, polyethylene glycol, 2-methoxy-1-propanol.

Ketone within the content of this disclosure is understood to be selected form the group consisting of acetone, methyl ethyl ketone, diethyl ketone, methyl isopropyl ketone, diisopropyl ketone, methyl isobutyl ketone, cyclopropyl methyl ketone, methyl tert-butyl ketone, 2-pentanone, cyclopentanone, 2-hexanone, cyclohexanone, 2-heptanone, 4-heptanone.

Ester within the content of this disclosure is understood to be selected form the group consisting of methyl formate, methyl acetate, ethyl acetate, n-propyl acetate, isopropyl acetate, n-butyl acetate, tert-butyl acetate, hexyl acetate, methyl propionate, γ-butyrolactone, benzoic acid ethylester, glycol diacetate and diethylene glycol diacetate.

Amide within the content of this disclosure is understood to be selected form the group consisting of N-methylformamide, N,N-dimethylformamide, N-methylacetamide, N,N-dimethylacetamide, N,N-diethylacetamide, N,N-dimethylpropionamide, N,N-dibutylformamide. N-methylpyrrolidone.

Nitrile within the content of this disclosure is understood to be selected form the group consisting of acetonitrile, propionitrile, benzonitrile and trimethylacetonitrile.

Halogenated solvent within the content of this disclosure is understood to be selected form the group consisting of methylene chloride, chloroform, carbon tetrachloride, 1,1-dichloroethylene, 1,2-dichloroethane, 1,1,1,-trichloroethane, 1,1,1,2-tetrachloroethane, 1,1,2,2-tetrachloroethane, chlorobenzene, o-dichlorobenzene, m-dichlorobenzene, p-dichlorobenzene, 1,2,3-trichlorobenzene, 1,2,4-trichlorobenzene, 4-chlorotoluene, trichloroacetonitrile, 2-chloroethanol, 2,2,2-trichloroethanol, 1-chloro-2-propanol, 2,3-dichloropropanol, 2-chloro-1-propanol in all isomeric forms, benzotrichloride, fluorobenzene, difluorobenzene in all its isomeric forms, 2,4,6-trifluorotoluene, 2-fluorobutanol, benzotrifluoride.

Carbonate within the content of this disclosure is understood to be selected form the group consisting of ethylene carbonate, propylene carbonate, dimethyl carbonate or diethyl carbonate.

A C-bearing solvent of the inventive process is any solvent adapted to largely solubilize or entirely solubilize all of the reagents chroman C1, gaseous compound comprising, essentially consisting of, or consisting of oxygen and copper catalyst. Such C-bearing solvent is to have both a hydrophilic character and a lipophilic character.

Such C-bearing solvent is selected from at least one of the group consisting of low aliphatic alcohol, namely from at least one C1-C8-alcohol including C1-C8-diols and C1-C8-triols, N,N-dimethylformamide, N,N-diethylformamide, N-methylpyrrolidone, ethylene carbonate, propylene carbonate, glycol ethers. C1-C5-alcohols are selected from the group consisting of methanol, ethanol, n-propanol, isopropyl alcohol, n-butanol, sec-butyl alcohol, isobutyl alcohol, tert.-butyl alcohol, 1-pentanol, isoamyl alcohol, 2-methyl-1-butanol, neopentyl alcohol, 2-pentanol, 3-pentanol, 3-methyl-2-butanol, 2-methyl-2-butanol, cyclopentanol, n-hexanol (1-hexanol), 2-methyl-1-pentanol, 3-methyl-1-pentanol, 4-methyl-1-pentanol, 2,2-dimethyl-1-butanol, 2,3-dimethyl-1-butanol, 3,3-dimethyl-1-butanol, 2-ethylbutan-1-ol, 2-hexanol, 3-hexanol, 3-methyl-2-pentanol, 4-methyl-2-pentanol in all isomeric forms, 3,3-dimethyl-2-butanol, 2-methyl-2-pentanol, 3-methylpentan-3-ol, 2,3-dimethyl-2-butanol, cyclohexanol, methylcyclopentanol, 1,3-dimethyl butanol, amylmethyl alcohol, methylisobutyl carbinol, 4-methyl-2-pentanol, tert-hexyl alcohol, n-heptanol, 2-heptanol, 3-heptanol, 3-ethyl-3-pentanol, n-octanol or 1-octanol, 2-octanol, 2-ethylhexanol, 1,2-ethanediol, 1,2-propanediol, 1,3-propanediol, 1,2-butanediol, 1,3 butanediol, 2,3-butanediol, 2-methyl-1,2-propanediol, 1,4-butanediol, 1,2-pentanediol, 1,3-pentanediol, 1,4-pentanediol, 1,5-pentanediol, 2,4-pentanediol, 2,2-dimethyl-1,3-propanediol, 3-methyl-2,4-pentanediol, 4-hydroxy-4-methyl-2-pentanol, 1,2,4-trihydroxybutane, 1,2,3-trihydroxybutane, triethyleneglycol, 1,6-hexane diol, 1,5 hexane diol, 1,4-hexane diol, 1,3-hexane diol, 2-methyl-2,4-pentane diol, pinacol, 2,3-dimethyl-2,3-butandiol, 1,2,5-hexane triol, 1,2,6-hexane triol, 2-methyl-2,3-butandiol, ethylene glycol monomethyl ether, ethylene glycol monoethyl ether, 2-ethoxyethanol, ethylene glycol monobutyl ether, 2-isopropoxyethanol, diethylene glycol, diethylene glycol monomethyl ether, diethylene glycol monoethyl ether, diethylene glycol diacetate, propylene glycol, 1,2-butylene glycol, triethylene glycol, glycerol, glycol diacetate and diethylene glycol diacetate, 2-methoxy-1-propanol.

Glycol ethers are for example ethylene glycol dimethyl ether, ethylene glycol diethyl ether, ethylene glycol dibutyl ether, diethylene glycol dimethyl ether, diethylene glycol diethyl ether, dipropylene glycole, trimethylene glycol dimethyl ether, trimethylene glycol diethyl ether, triethylene glycol dimethylether.

In a further embodiment, a solvent mixture comprising water, an alcohol comprising from 1 to 8 carbon atoms, preferably an alcohol comprising from 1 to 6 carbon atoms, and a hydrocarbon was revealed to improve the rate and/or the yield of the inventive process. The reason for that is not yet entirely clear. It may be related to the fact, that an alcohol in water increases the capacity of said mixture to dissolve small amounts of hydrocarbon in the alcohol/water phase. A further developed embodiment of the invention thus is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and said solvent mixture comprising water, an alcohol comprising from 1 to 8 carbon atoms and a hydrocarbon, preferably water, an alcohol comprising from 1 to 6 carbon atoms and a hydrocarbon, more preferably said solvent mixture comprising water, an alcohol comprising from 1 to 8 carbon atoms and an aromatic hydrocarbon, and most preferably said solvent mixture comprising water, an alcohol comprising from 1 to 6 carbon atoms and an aromatic hydrocarbon.

A substantial goal of the inventive process is not only to be free of side products to the utmost extent possible but also to reduce or completely avoid trace amounts of reagents or reagent portions like copper ions, chlorine ions, organic chlorine compounds etc. This was found to be achieved by means of using an alcohol in the solvent mixture, preferably a secondary alcohol and even more preferred a secondary alcohol having at least six carbon atoms. This finding is reflected by an embodiment where the at least two solvents of the solvent mixture comprise as organic solvent at least one primary alcohol or at least one secondary alcohol or a mixture of at least one primary and at least one secondary alcohol, with said secondary alcohol, preferably being an alcohol having at least six carbon atoms and more preferably having at least seven carbon atoms.

In a further embodiment of the invention the weight ratio of the organic solvent to water ranges from 0,01:1 to 499:1, preferably from 0,1:1 to 450:1, further preferred from 0,4:1 to 350:1, still further preferred from 1:1 to 300:1, in a further embodiment form 1,1:1 to 200:1, in a still further preferred variant from 2,9:1 to 175:1, in another preferred embodiment from 3,1:1 to 150:1, more preferably from 4,3:1 to 100 to 1, yet more preferably from 5:1 to 70:1, still further preferred from 6:1 to 31.4:1, more preferably from 7:1 to 29:1, in a further developed embodiment form 7,5:1 to 21,3:1, yet in another embodiment from 7,9:1 to 19,6:1, still further preferred form 10:1 to 17,4:1, further preferred from 11,6:1 to 14:1, and most preferred 10,59 to 13,73:1.

Many of the previously disclosed embodiments stress a short reaction time ranging from 2 h to 8 h, preferably from 2 h to 7 h and even more preferably from 2 h to 6 h (cf. examples 905 (CN58), 1032 (CN59), 879 (CN60), 1021 (CN61), 1074 (CN62), 941 (CN63), 877 (CN64), 1054 (CN65),1052 (CN66), 1086 (CN67)). One embodiment of the invention thus defines a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2) and said process of oxidation being realized within less than 48 h, preferably within a time ranging from 2 h to 8 h, more preferably within a time ranging from 2 h to 7 h, even more preferably from 4 h to 6 h and most preferably from 4,75 h to 6 h including 4,8 h and 5 h.

Another advantageous feature of the inventive process is that high yields and short reaction times can even be realized at moderate temperatures (cf. examples 1024 (CN68), 877 (CN69), 883 (CN70), 941 (CN71), 942 (CN72), 1060 (CN73), 905 (CN74), 988 (CN75), 894 (CN76), 1054 (CN77), 879 (CN78), 994 (CN79), 1032 (CN80)). This is less energy-intensive and thus cost-saving. Said embodiment defines a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being conducted at a temperature ranging from 2° C. to 170° C., preferably form 10° C. to 60° C. more preferred from 15° C. to 55° C., even more preferred from 20° C. to 50° C. and mostly preferred from 25° C. to 40° C. including 23° C. This also holds

Said embodiment defines a further process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being conducted at a temperature ranging from 2° C. to 170° C., preferably form 10° C. to 60° C. more preferred from 10° C. to 55° C., even more preferred from 15° C. to 40° C. and mostly preferred from 15° C. to 25° C. including 23° C.

Both temperature and reaction time influence the amount of quinone C30 formed. However, not only the amount of quinone C30 but also the quantity of trace products or reagent traces like e.g. Cu-ions, organic chlorine or chloride were shown to change with reaction time and reaction temperature.:

From R,R.R-α-tocopherol C5 reacted semi-batchwise one obtains after distillation or after extraction and solvent removal a quinone preparation with components as shown in to Table 1a and Table 1 b:

TABLE 1a

| | | R,R,R-α- | | | | | |
|---|---|---|---|---|---|---|---|
| | | tocopherol | | | | | |
| | | quinone | Organic | | Cu | | Total |
| | Exam- | C33 Yield | chlorine | Chloride | ions | Temp. | time |
| CN | ple | [%] | [ppm] | [ppm] | [ppm] | [° C.] | [h] |
| 81 | 1042 | 99 | 88 | 12 | 8 | 25 | 6 |

The examples of Table 1 b serve as comparison with respect to temperature and reaction time dependent trace formation. However, they are nevertheless inventive examples of the invention when no emphasize is given to temperature and reaction time dependent trace formation.

TABLE 1b

| | | R,R,R-α- | | | | | |
|---|---|---|---|---|---|---|---|
| | | tocopherol | | | | | |
| | | quinone | Organic | | Cu | | Total |
| | Exam- | C33 Yield | chlorine | Chloride | ions | Temp. | time |
| CN | ple | [%] | [ppm] | [ppm] | [ppm] | [° C.] | [h] |
| 82 | 1032 | 100 | 149 | 21 | 31 | 40 | 5 |
| 83 | 1036 | 92 | 356 | 34 | 40 | 55 | 5 |

From R,R,R-α-tocopherol C5 reacted batchwise one obtains after distillation or after extraction and solvent removal a quinone preparation with components as shown in Table 2a and Table 2b:

TABLE 2a

| | | R,R,R-α- | | | | | |
|---|---|---|---|---|---|---|---|
| | | tocopherol | | | | | |
| | | quinone | Organic | | Cu | | |
| | Exam- | C33 Yield | chlorine | Chloride | ions | Temp. | Time |
| CN | ple | [%] | [ppm] | [ppm] | [ppm] | [° C.] | [h] |
| 84 | 886 | 96 | 70 | 80 | 95 | 10 | 8 |
| 85 | 877 | 99 | 27 | 9 | 5 | 15 | 6 |
| 87 | 1080 | 95 | 44 | 32 | 30 | 25 | 5 |
| 88 | 905 | 100 | 77 | 21 | 13 | 25 | 6 |
| 89 | 1054 | 95 | 60 | 26 | 12 | 25 | 5 |
| 90 | 879 | 99 | 61 | 9 | 11 | 25 | 6 |

The examples of Table 2b serve as comparison with respect to temperature and reaction time dependent trace formation. However, they are nevertheless inventive examples of the invention when no emphasize is given to temperature and reaction time dependent trace formation.

TABLE 2b

| | | R,R,R-α- | | | | | |
|---|---|---|---|---|---|---|---|
| | | tocopherol | | | | | |
| | | quinone | Organic | | Cu | | |
| | Exam- | C33 Yield | chlorine | Chloride | ions | Temp. | Time |
| CN | ple | [%] | [ppm] | [ppm] | [ppm] | [° C.] | [h] |
| 91 | 1024 | 94 | 100 | 100 | 105 | 10 | 9 |
| 92 | 1040 | 96 | 293 | 27 | 23 | 25 | 23 |
| 93 | 1010 | 95 | 250 | 70 | 100 | 40 | 5 |

One observes from these tables, that low amounts of organic chlorine, chloride and Cu ion can be obtained provided one observes an appropriate reaction time and reaction temperature. One embodiment, being adapted to low amounts of organic chlorine, chloride and Cu-ions is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 10° C. to 50° C. and within a time ranging from 2 h to 7 h.

Another embodiment, being adapted to low amounts of organic chlorine, chloride and Cu-ions is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 10° C. to 25° C. and within a time ranging from 2 h to 7 h.

Yet another embodiment, being adapted to low amounts of organic chlorine, chloride and Cu-ions is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 20° C. to 50° C. and within a time ranging from 2 h to 7 h.

A still further developed form of the previous embodiment is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 20 to 40° C. and within a time 15 ranging from 2 h to 7 h including 6 h.

An additional embodiment, being adapted to low amounts of organic chlorine, chloride and Cu-ions is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 10° C. to 50° C. and within a time ranging from 2 h to 8 h.

Yet a further embodiment, being adapted to low amounts of organic chlorine, chloride and Cu-ions is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 10° C. to 50° C. and within a time ranging from 5 h to 8 h.

Finally a highly preferred embodiment, being adapted to low amounts of organic chlorine, chloride and Cu-ions is a process for the oxidation of at least one chroman C1, in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2), said process being realized at a temperature ranging from 10° C. to 25° C. and within a time ranging from 5 h to 8 h.

This last embodiment as can be seen from tables 1a and 2a gives the lowest amount of organic chlorine, chloride and Cu ions compared to tables 1b and 2b.

A substantial part of the invention is a composition comprising: a) at least one chroman C1

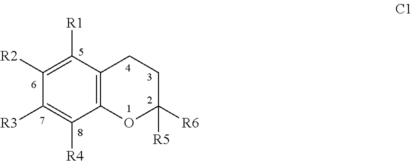

with R1, R3, R4, R5 being H or CH₃, R2 being OH, OAc, OCO—C₁-C₁₈-alkyl, and R6 being alkyl, alkenyl and/or at least one quinone C30

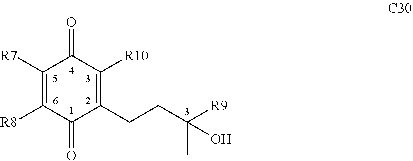

with R7, R8, R10 being H or CH₃; R9 being alkyl, alkenyl; b) a solvent mixture comprising at least two solvents or a C-bearing solvent; c) a copper catalyst, said copper catalyst exhibiting the oxidation state (+1) or (+2); d) a gaseous compound comprising, essentially consisting or consisting of oxygen; said composition being preferably obtained by a process as disclosed in any one of the previously mentioned embodiments. Only such composition was shown to contain a huge amount of chroman C1 and to be the starting point for selectively obtaining a quinone C30 in high yield and short reaction time. Said same composition was shown to be mainly composed of a huge amount of quinone C30 without showing by products. For sake of clarity, said inventive composition is understood to contain the components as indicated. However, the amount of chroman C1 changes with time depending on the moment at which a sample is to be taken from said composition. If such sample is taken prior to starting the inventive process, the amount of chroman C1 is the highest and the amount of quinone C30 is zero. At the end of the inventive process, the amount of chroman C1 in the composition is either zero or only traces thereof remain and the amount of quinone C30 is the highest possible. In a preferred embodiment, it ranges from 85 to 100 percent of the molar amount of chroman C1 initially present in the composition. During the inventive process, the composition contains different amounts of chroman C1 and quinone C30 depending on the time of the inventive process at which a composition sample was taken and analyzed. Since the inventive process for several embodiments of this invention can be stopped at any time by simply turning of the stirring means, thus revealing any molar ratio of chroman C1 to quinone C30 (viz. ranging from chroman C1:quinone C30 equal to 0 mol %: 100 mol % to 100 mol % to 0 mol %), any composition comprising one of the previously indicated chroman C1/quinone C30 ratios and the components b) to d) is understood to be a composition of the invention.

Another embodiment of the invention further characterizes the inventive composition. It is the composition as previously mentioned, viz. a composition comprising: a) at least one chroman C1 and/or at least one quinone C30; b) a solvent mixture comprising at least two solvents or a C-bearing solvent; c) a copper catalyst, said copper catalyst exhibiting the oxidation state (+1) or (+2); d) a gaseous compound comprising, essentially consisting or consisting of oxygen; said composition being preferably obtained by a process according to any one of the previously mentioned embodiments. Said embodiment is further defined such that the gaseous compound in the composition is in the form of gas bubbles, the amount of which being higher than that amount, which is obtained, when a) to c) are combined and stored under ambient air, preferably higher than that amount, which is obtained, when a) to c) are combined and stirred under ambient air. As can be seen, such embodiment necessarily requires a certain amount of gas bubbles to be included. Said gas bubbles of the gaseous compound contribute to obtain one embodiment of the inventive composition exhibiting or able to form a high amount of quinone C30 while simultaneously avoiding the amount of side products formed out of the chroman C1.

Another aspect of the invention deals with a process of transforming the inventive composition into a quinone preparation. For certain food and pharmaceutical applications, the inventive composition per se and in particular the quinone C30 have to comply with certain specifications as are required from national and/or multinational administrative bodies. Said specifications require to limit the amount of a combination of byproducts or reagent traces to a predefined extend.

This need is addressed in one favorable embodiment by a process for obtaining a quinone preparation comprising the steps: i) removing one solvent from the solvent mixture comprising at least two solvents of the inventive composition, or removing the C-bearing solvent of the inventive composition; with optionally adding hydrochloric acid prior or during removing one solvent from the solvent mixture or prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation means, the diameter of the surface of said separation means being larger than the height of said separation means; iv) optionally subjecting the remainder from step iii) to a further distillation. By subjecting the inventive composition to a process as described supra one obtains inventive quinone preparations having the following characteristics:

From R,R.R-α-tocopherol C5 reacted semi-batchwise one obtains after having applied it to the separation means a quinone preparation comprising trace amounts as given in Table 3:

TABLE 3

| | | With raw | Organic | | Cu |
|---|---|---|---|---|---|
| | Exam- | material from | chlorine | Chloride | ions |
| CN | ple | example | [ppm] | [ppm] | [ppm] |
| 94 | 1044 | 1042 cf. supra | 16 | <3 | <3 |
| 95 | 1033 | 1032 cf. supra | 76 | <3 | <3 |
| 96 | 1038 | 1036 cf. supra | 210 | <3 | <3 |

From R,R.R-α-tocopherol C5 reacted batchwise one obtains after having applied it to the separation means a quinone preparation comprising trace amounts as given in Table 4:

TABLE 4

| | | With raw | Organic | | Cu |
|---|---|---|---|---|---|
| | Exam- | material from | chlorine | Chloride | ions |
| CN | ple | example | [ppm] | [ppm] | [ppm] |
| 97 | 895 | 886 cf. supra | 12 | <1 | <3 |
| 98 | 1027 | 1024 cf. supra | 20 | <3 | <3 |
| 99 | 1092 | 1091 | 5 | <3 | 7 |
| 100 | 880 | 877 cf. supra | 14 | <1 | <3 |
| 101 | 1019 | 1014 | 15 | <3 | <3 |
| 102 | 1056 | 1053 (CN2) | 18 | <3 | <3 |
| 103 | 908 | 905 cf. supra | 32 | <1 | <3 |
| 104 | 909 | 906 | 34 | <1 | <3 |
| 105 | 1049 | 1040 cf. supra | 170 | <3 | <3 |
| 106 | 1012 | 1010 cf. supra | 65 | <3 | <3 |
| 107 | 1057 | 1054 cf. supra | 9 | <3 | <3 |
| 108 | 885 | 879 cf. supra | 36 | <1 | <3 |
| 109 | 1087 | 1086 | 7 | <3 | <3 |

From rac-α-tocopherol C3 with R2 being OH reacted batchwise, one obtains after having applied it to the separation means a quinone preparation comprising trace amounts as given in Table 5:

TABLE 5

| | | With raw | Organic | | Cu |
|---|---|---|---|---|---|
| | Exam- | material from | chlorine | Chloride | ions |
| CN | ple | example | [ppm] | [ppm] | [ppm] |
| 110 | 1008 | 994 | 47 | <3 | <3 |

It can be seen from Tables 3 to 5 that the separation means considerably reduces the amount of organic chlorine, of chloride and of Cu ions yielding a quinone preparation of the invention.

In another inventive embodiment, this need of being compliant with administrative specifications is addressed by a process for obtaining a quinone preparation comprising the steps: i) removing one solvent from the solvent mixture comprising at least two solvents of the inventive composition, or removing the C-bearing solvent of the inventive composition; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) to another distillation step; iv) optionally subjecting the remainder from step iii) to a further distillation. Proceeding this way, one obtains inventive quinone preparations having the following characteristics:

From R,R.R-α-tocopherol C5 reacted semi-batchwise one obtains after having submitted it to distillation an inventive quinone preparation comprising trace amounts as given in Table 6:

TABLE 6

| | | With raw | Organic | | Cu |
|---|---|---|---|---|---|
| | Exam- | material from | chlorine | Chloride | ions |
| CN | ple | example | [ppm] | [ppm] | [ppm] |
| 111 | 1043 | 1042 cf. supra | 51 | <3 | 2 |
| 112 | 1034 | 1032 cf. supra | 115 | 5 | 14 |
| 113 | 1039 | 1036 cf. supra | 321 | 9 | 24 |

From R,R.R-α-tocopherol C5 reacted batchwise one obtains after having submitted it to (further) distillation an inventive quinone preparation comprising trace amounts as given in Table 7.

TABLE 7

| | | With raw | Organic | | Cu |
|---|---|---|---|---|---|
| | Exam- | material from | chlorine | Chloride | ions |
| CN | ple | example | [ppm] | [ppm] | [ppm] |
| 114 | 887 | 886 cf. supra | 77 | 8 | 41 |
| 115 | 1028 | 1024 cf. supra | 86 | 24 | 39 |
| 116 | 878 | 877 cf. supra | 18 | <1 | <3 |
| 117 | 1016 | 1014 | 38 | <3 | 3 |
| 118 | 910 | 905 cf. supra | 74 | 7 | 22 |
| 119 | 911 | 906 | 77 | 3 | 11 |
| 120 | 1048 | 1040 cf. supra | 239 | 11 | 13 |
| 121 | 1011 | 1010 cf. supra | 131 | 29 | 43 |
| 122 | 1055 | 1054 cf. supra | 56 | 3 | 6 |
| 123 | 881 | 879 cf. supra | 53 | <1 | <3 |
| 124 | 1090 | 1089 | 23 | <3 | 3 |

From rac-α-tocopherol C3 reacted semi-batchwise one obtains after having submitted it to distillation an inventive quinone preparation comprising trace amounts as given in Table 8

TABLE 8

| | | With raw | Organic | | Cu |
|---|---|---|---|---|---|
| | Exam- | material from | chlorine | Chloride | ions |
| CN | ple | example | [ppm] | [ppm] | [ppm] |
| 125 | 992 | 990 | 79 | <3 | <3 |

It can be seen from Tables 6 to 8 that distillation reduces the amount of organic chlorine, of chloride and of Cu ions yielding a quinone preparation of the invention. However, its performance is not as pronounced as with the separation means.

In yet another embodiment, this need to be compliant with administrative specifications is addressed by a process for obtaining a quinone preparation comprising the steps: i) removing one solvent from the solvent mixture comprising at least two solvents of the inventive composition, or removing the C-bearing solvent of the inventive composition; with optionally adding hydrochloric acid prior or during removing one solvent from the solvent mixture or adding hydrochloric acid prior or during removing the C-bearing solvent; iia) distilling off the remaining solvent(s) or iib) degassing the composition or; iic) distilling off remaining solvent(s) and degassing the composition; iii) applying the composition of step iia), step iib) or step iic) onto a separation column; iv) optionally subjecting the remainder from step iii) to a further distillation. By subjecting the inventive composition to a process as described supra one obtains inventive quinone preparations having the following characteristics:

From R,R.R-α-tocopherol C5 reacted semi-batchwise one obtains after having applied it to the separation column a quinone preparation comprising trace amounts as given in Table 9.:

TABLE 9

| Example | CN |
|---|---|
| 1046 | 126 |

| | |
|---|---|
| organic chlorine [ppm] | 73 |
| chloride [ppm] | 3 |
| Cu ions [ppm] | <3 |

As can be seen from the tables above, the embodiment using in step iii) another distillation step is mostly suited, if the amount of residual Cu-ions is not required to be very low. On the other hand, with likewise low Cu ion concentration and low chlorine concentration, a separation column can fit this need. Resins or solid supports of separation columns are expensive compared to distillation. Reducing the amount of resin or solid support used, reduces process costs. This is achieved with a separation means, the diameter of the surface of which being larger than the height thereof. As can be seen from the tables above, even with a reduced amount of resin or solid support as used in the separation means, similar or even better results with respect to residual amount of organic chlorine, chloride and Cu ions are obtained, which is surprising.

During the inventive process for the oxidation of chroman C1 in the presence of a copper catalyst as well as in the process for obtaining a quinone preparation, in some but not all embodiments a copper catalyst depletion was observed with time. This depletion is cumulative, if one and the same catalyst sample is employed repeatedly, regardless whether it is used batchwise or semi batchwise. Reduced amount of copper catalyst, when passing a certain threshold, however also reduces the reaction rate and increases reaction costs due to supplementing the reaction mixture with additional fresh copper catalyst. In order to avoid this, several measures are suitable and being reflected by the following three embodiments.

When the chroman C1 used in the inventive oxidation process and the quinone C30 used in the process for obtaining a quinone preparation can stand acidic conditions, said process for obtaining a quinone preparation by means of a separation means comprises the steps: i) removing one solvent from the solvent mixture comprising at least two solvents of the inventive composition, or removing the C-bearing solvent of the inventive composition; adding hydrochloric acid prior or during removing one solvent from the solvent mixture or adding hydrochloric acid prior or during removing the C-bearing solvent; iia) distilling off remaining solvent(s); or iib) degassing the composition; or iic) distilling off remaining solvent(s) and degassing the composition; iii) applying the composition of step iia), step iib) or step iic) onto a separation means, the diameter of the surface of said separation means being larger than the height of said separation means; iv) optionally subjecting the remainder from step iii) to a further distillation.

When the chroman C1 used in the inventive oxidation process and the quinone C30 used in the process for obtaining a quinone preparation can stand acidic conditions, said process for obtaining a quinone preparation by means of a distillation step comprises the steps: i) removing one solvent from the solvent mixture comprising at least two solvents of the inventive composition or removing the C-bearing solvent of the inventive composition; adding hydrochloric acid prior or during removing one solvent from the solvent mixture or adding hydrochloric acid prior or during removing the C-bearing solvent; iia) distilling off remaining solvent(s); or iib) degassing the composition; or iic) distilling off remaining solvent(s) and degassing the composition; iii) applying the composition of step iia), step iib) or step iic) to another distillation step; iv) optionally subjecting the remainder from step iii) to a further distillation.

When the chroman C1 used in the inventive oxidation process and the quinone C30 used in the process for obtaining a quinone preparation can stand acidic conditions, said process for obtaining a quinone preparation by means of a separation column comprises the steps: i) removing one solvent from the solvent mixture comprising at least two solvents of the inventive composition, or removing the C-bearing solvent of the inventive composition; adding hydrochloric acid prior or during removing one solvent from the solvent mixture or adding hydrochloric acid prior or during removing the C-bearing solvent; iia) distilling off the remaining solvent(s); or iib) degassing the composition or; iic) distilling off remaining solvent(s) and degassing the composition; iii) applying the composition of step iia), step iib) or step iic) onto a separation column; iv) optionally subjecting the remainder from step iii) to a further distillation.

By each of these three embodiments, viz. by adding hydrochloric acid, degraded or used copper catalyst can be regenerated or recycled for reuse.

Not every chroman C1 nor every quinone C30 easily supports acidic conditions without experiencing degradation to some extent. The three embodiments mentioned previously are less favorable for such acid-labile chromans C1 and/or acid-labile quinones C30. This drawback can be addressed by the following embodiments. It also gives the advantage to recover or to recycle components like solvents in a purity sufficient to reuse them in said process. Likewise, it is suitable to reconvert trace components like e.g. copper oxochlorides into reagents of the inventive process (like CuCl₂) for the selective oxidation of at least one chroman C1 or in the process for obtaining a quinone preparation. The following embodiment comprises a separation means.

One embodiment adapted to acid labile chromans C1, quinones C30 in a solvent mixture comprising at least two solvents discloses a process for obtaining a quinone preparation comprising the steps: i) removing one solvent from the solvent mixture comprising at least two solvents of the inventive composition; ia) reducing the volume of the removed one solvent and/or; ib) adding hydrochloric acid to said removed one solvent; ic) storing or reinjecting the thus obtained mixture of step ia) or ib) for further use in the inventive process for the oxidation of at least one chroman C1, or instead of steps ia) to ic); id) adding hydrochloric acid to said removed one solvent and/or; ie) reducing the volume of the mixture obtained in step id); if) storing or reinjecting the thus obtained mixture of step id) or ie) for further use in the inventive process for the oxidation of at least one chroman C1; iia) distilling off remaining solvent(s) not removed in step i), or iib) degassing the composition; or iic) distilling off remaining solvent(s) not removed in step i) and degassing the composition; iii) applying the composition of step iia), step iib) or step iic) onto a separation means, the diameter of the surface of said separation means being larger than the height of said separation means; iv) optionally subjecting the remainder from step iii) to a further distillation.

The previous embodiment in step iii) can have, instead of a separation means, either another distillation step or a separation column. This provides another two embodiments, which only differ in step iii) from the previous one. In one of these two embodiments, step iii) reads: “applying the composition of step iia), step iib) or step iic) to another distillation step;”, in the other one of them, step iii) reads: “applying the composition of step iia), step iib) or step iic) onto a separation column;”. These two embodiments, in addition to the one previously mentioned, are also an integral part of the invention.

A “separation means, the diameter of the surface of said separation means being larger than the height of said separation means” is understood to be a recipient the diameter of its surface being larger than its height and comprising a or supplemented with a solid support synonymous to resin. The term “separation means, the diameter of the surface of said separation means being larger than the height of said separation means” also comprises an embodiment made of recipient comprising or supplemented with a solid support, where only the diameter of the surface given by the solid support is larger than the height of said solid support. Likewise, the term “separation means the diameter of the surface of said separation means being larger than the height of said separation means” includes an embodiment where both the diameter of the recipient surface of said separation means being larger than the recipient's height and the diameter of the surface given by the solid support being larger than the height of said solid support. “Surface” irrespective of whether it belongs to the recipient or to the solid support means an area perpendicular to the respective height.

The characteristic feature of said separation means is its dimensioning. The diameter of the surface of said separation means is larger than the height of said separation means. As a consequence, less solid support or resin can be placed in the recipient and is used for a separation task compared to the amount used e.g. in a separation column. With a lower amount of solid support or resin being used in the separation means, one would estimate separation results to be less favorable than e.g. in a separation column. However, against expectation, the contrary was observed as indicated supra.

Said solid support of the separation means is any support suited to separate chemical entities like molecules, ions according to at least one of polarity, size, charge, chirality, when said chemical entities are applied thereto in a solvent or solvent mixture. In one embodiment, the solid support is selected from at least one of silica, silica based material also named modified silica viz. coated with inorganic or organic molecules, zeolite, aluminum oxide, alumina silicates, carbon, carbon based materials, carbohydrate including carbohydrate soft gels, carbohydrates crosslinked with agarose or acrylamides, polymeric organic materials including crosslinked organic polymers like polymeric resins or ion exchange materials, methacrylic resins, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA) preferably being silica.

A “separation column” is understood to be a tube, pipe or tubing comprising or supplemented with a solid support synonymous to resin, the diameter of the surface of the tube, pipe or tubing being smaller than or equal to the height thereof. A “separation column” is also understood to be a tube, pipe, tubing, comprising or supplemented with a solid support synonymous to resin, where the diameter of the surface given by the solid support is smaller than or equal to the height of said solid support in the tube, pipe or tubing. Likewise a “separation column” is understood to be a tube, pipe or tubing comprising or supplemented with a solid support synonymous to resin, where both the diameter of the surface of the tube, pipe or tubing being smaller than or equal to the height thereof and the diameter of the surface of the solid support being smaller or equal to the height of the solid support. “Surface” irrespective of whether it belongs to the recipient or to the solid support means an area perpendicular to the respective height.

Said solid support of the separation column is any support suited to separate chemical entities like molecules, ions according to at least one of polarity, size, charge, chirality, when said chemical entities are applied thereto in a solvent or solvent mixture. In one embodiment, the solid support is selected from at least one of silica, silica based material also named modified silica viz. coated with inorganic or organic molecules, zeolite, aluminum oxide, alumina silicates, carbon, carbon based materials, carbohydrate including carbohydrate soft gels, carbohydrates crosslinked with agarose or acrylamides, polymeric organic materials including crosslinked organic polymers like polymeric resins or ion exchange materials, methacrylic resins, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica.

Separation capacity for quinone C30 from byproducts and/or reagent traces was found to also be influenced by the particle size of the solid support employed in the separation means and/or in the separation column. Persuasive results were obtained, when the solid support, preferably silica, has a particle size ranging from 5 μm to 1000 μm, preferably from 10 μm to 150 μm, more preferably ranging from 30 μm to 100 μm and most preferably ranging from 40 μm to 63 μm; and a mean pore size ranging from 1 to 100 nm. The particle sizes and pore sizes are to be taken as indicated by the provider of the solid support.

In one embodiment the separation means or the separation column is operated in a batch mode. Batch mode means sample to applied onto the separation means or the separation column, separation is realized, optionally the separation means is regenerated and a subsequent sample is applied.

The separation means or the separation column in one embodiment is operated at ambient pressure.

In another embodiment the separation means or the separation column respectively is operated under pressure, either under low pressure or under high pressure but not under ambient pressure. For operation under pressure the granulometry required for the solid support is a little bit different to what is needed when working under ambient pressure or in other words a different granulometry will yield a different pressure to form during separation.

Pressure besides ambient pressure as understood within this specification is any pressure ranging from 1,1×10⁵ pascal to 150×10⁵ pascal. Ambient pressure is any pressure measured under atmospheric conditions without applying any pressure means, viz a pressure ranging from 0,9×10⁵ pascal to 1,1×10⁵ pascal depending on the actual weather conditions. Low pressure within this specification means 1,1×10⁵ pascal to 10×10⁵ pascal. High pressure within this specification is understood to be any value ranging from 10×10⁵ pascal to 150×10⁵ pascal.

A further embodiment of the invention seeks protection for a process for obtaining a quinone preparation comprising the steps: i) removing one solvent from the solvent mixture comprising at least two solvents of the composition of the invention, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation means, the diameter of the surface of said separation means being larger than the height of said separation means; the separation means comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica and said solid support having a particle size ranging from 50 μm and up to 1000 μm, preferably from 200 μm up to 500 μm, particularly preferred from 250 μm up to 350 μm and a pore size from 1 nm to 100 nm; iv) optionally subjecting the remainder from step iii) to a further distillation, preferably at least one further distillation. Said process using the separation means is particularly adapted for runs with low pressure and gives good purification results both with respect to chlorine traces and Cu traces.

Yet another embodiment of the invention seeks protection for a process for obtaining a quinone preparation comprising the steps: i) removing one solvent from the solvent mixture comprising at least two solvents of the composition of the invention, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation means, the diameter of the surface of said separation means being larger than the height of said separation means; said separation means comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid, malic acid, or nitrilotriacetic acid (NTA), preferably being silica and having a particle size ranging from 5 to 50 μm and a pore size from 1 to 100 nm; iv) optionally subjecting the remainder from step iii) to a further distillation. This embodiment of the inventive process using a separation means provides the opportunity to satisfactorily separate trace amounts of chlorine or copper ions under high pressure with the separation means.

Provided further reagents or compounds for whatever additional reason are required to be within the process for the oxidation of at least one chroman C1 or in the composition comprising at least one chroman C1 and/or at least one quinone C30, a separation means may not be efficient in substantially removing all traces or by-products or the like of this process or of the inventive composition. This need is addressed by two further embodiments one of which is:

For use at ambient or low pressure a process for obtaining a quinone preparation comprising the steps: i) removing one solvent from the solvent mixture comprising at least two solvents of the composition of the invention, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation column; the separation column comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid, malic acid, or nitrilotriacetic acid (NTA), preferably being silica and having a particle size ranging from 50 μm and up to 1000 μm, preferably from 200 to 500 μm, particularly preferred 250-350 μm and a pore size ranging from 1 nm to 100 nm; iv) optionally subjecting the remainder from step iii) to a further distillation. This embodiment of the inventive process is adapted for removing a larger variety of traces or by products under low pressure.

The other embodiment adapted to be used under high pressure is a process for obtaining a quinone preparation comprising the steps: i) removing one solvent from the solvent mixture comprising at least two solvents of the composition of the invention, or removing the C-bearing solvent of the composition of the invention with optionally adding hydrochloric acid prior or during removing one solvent from the solvent mixture or with optionally adding hydrochloric acid prior or during removing the C-bearing solvent; iia) distilling off the remaining solvent(s); or iib) degassing the composition; or iic) distilling off remaining solvent(s) and degassing the composition; iii) applying the composition of step iia), step iib) or step iic) onto a separation column; the separation column comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica and having a particle size ranging from 5 μm to 50 μm and a pore size ranging from 2 nm to 50 nm; iv) optionally subjecting the remainder from step iii) to a further distillation. This embodiment of the inventive process gives access to removing a greater variety of trace compounds or byproducts under high pressure.

Along the experiments realized it was found that the solvent, in which the solid support was suspended or immersed has an impact on the separation pattern of the solid support. Good separation conditions were achieved, when the solid support is suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide; formamide, dimethylformamide and water, preferably in a hydrocarbon and most preferably in n-hexane, n-heptane or cyclohexane, and the slurry thus obtained is applied to the separation means or to the separation column.

Aliphatic hydrocarbons, aromatic hydrocarbons, alcohols are as defined supra for one solvent of the solvent mixture comprising at least two solvents or for the C-bearing solvent.

A halogenated hydrocarbon is selected from the group consisting of dichloromethane, chloroform, perchloroethylene, chlorobenzene, dichlorobenzene, difluorobenzene in all its isomeric forms, benzotrifluoride, fluorinated lower alkanes.

A carboxylic acid is meant to be selected from the group consisting of formic acid, acetic acid, propionic acid.

An ester as understood within this disclosure is selected from the group of formates, acetates or propionates of methanol, ethanol, propanol, isopropanol, butanol, as for instance methyl formate, ethylformate, methyl acetate, ethyl acetate, propyl acetate, isopropyl acetate, butyl acetate, isobutyl actetate, methyl propionate.

An ether as meant within this specification is selected from the group consisting of dimethyl ether, diethyl ether, methyl ethylether, di-n-propyl ether, diisopropyl ether, tert-butyl methyl ether, dibutyl ether, anisole, tetrahydrofuran, 2-methyltetrahydrofuran, dioxane, ethylene glycol monomethyl ether, ethylene glycol dimethyl ether, ethylene glycol monoethyl ether, ethylene glycol diethyl ether, ethylene glycol monoisopro-pyl ether, dipropylene glycole, ethylene glycol monobutyl ether, ethylene glycol dibutyl ether, di-ethylene glycol monomethyl ether, diethylene glycol dimethyl ether, diethylene glycol monoethyl ether, diethylene gylcol diethyl ether, diethylene glycol diacetate, 2-methoxy-1-propanol.

A ketone as understood within this invention is selected from the group consisting of acetone, butanone, methyl ethyl ketone, diethyl ketone, diisopropyl ketone, isopropyl methyl ketone, isobutyl methyl ketone, methyl tert-butyl ketone, 2-pentanone, cyclopentanone, 2-hexanone, cyclohexanone, 2-heptanone, 4-heptanone.

An acetal is selected from the group consisting of formaldehyde dimethylacetal, formaldehyde diethylacetal, acetaldehyde dimethyl acetal, acetaldehyde diethylacetal, propionaldehyde dimethyl acetal, propionaldehyde diethylacetal.

A ketal of this disclosure is meant to be selected from the group consisting of 2,2-dimethoxypropane, 2,2-diethoxypropane.

A nitrile of this specification is selected from the group consisting of acetonitrile, propionitrile, butyronitrile, benzonitrile.

The experiments realized revealed in a further defined embodiment, that the solvent, in which the solid support was suspended or immersed has an impact on the separation pattern of the solid support. Good separation conditions were achieved, when the solid support of the previous embodiment, having a particle size below 50 to 100 μm, and a mean pore size ranging from 1 to 100 nm, is suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide; formamide, dimethylformamide, and water, preferably in a hydrocarbon and most preferably in n-hexane, n-heptane or cyclohexane, and the slurry thus obtained is applied to the separation means or to the separation column.

However, for particles being larger than 50 μm to 100 μm another precision of the penultimate embodiment was found to be more adapted. Good separation conditions were achieved, when the solid support of the penultimate embodiment, having a particle size above 50 to 100 μm, and a mean pore size ranging from 1 to 100 nm, is applied in dry form to the separation means or to the separation column and a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide, and water, preferably in a hydrocarbon and most preferably in n-hexane or n-heptane thereafter is applied to the separation means or to the separation column.

It is to be noticed that for particles having a particle size ranging from 50 to 100 μm and a mean pore size ranging from 1 to 100 nm, all of the previously mentioned three embodiments are adapted to be used.

A further embodiment for obtaining a quinone preparation is an inventive process, wherein the composition after step iia), step iib) or step iic) is dissolved or suspended in a diluting solvent or diluting solvent mixture selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and the diluted composition thus obtained is subjected to step iii).

The different types of diluting solvent indicated above have the meaning as given supra for the suspending solvent.

Quinone preparations having low amounts of trace compounds like organic chlorine, chloride and copper ions were obtained, when the suspending solvent for the solid support and the diluting solvent are not identical.

This is reflected by a process for obtaining a quinone preparation comprising the steps: i) removing one solvent from the solvent mixture comprising at least two solvents of the inventive composition, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation means, the diameter of the surface of said separation means being larger than the height of said separation means; the separation means comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica; the solid support, preferably silica, having a particle size ranging from 5 μm to 1000 μm, preferably from 10 μm to 150 μm, more preferably ranging from 30 μm to 100 μm and most preferably ranging from 40 μm to 63 μm; and a mean pore size ranging from 1 to 100 nm; the solid support being suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and the slurry thus obtained being applied to the separation means; then dissolving or suspending the composition after step iia), step iib) or step iic) in a diluting solvent or solvent mixture selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and applying the diluted composition thus obtained onto the separation means (step iii), with the suspending solvent or mixture of suspending solvents being different to the diluting solvent or the diluting solvent mixture; iv) optionally subjecting the remainder from step iii) to a further distillation.

However, when suspending solvent and diluting solvent are of the same nature, likewise good results were obtained.

This is taken into account by a process for obtaining a quinone preparation comprising the steps: i) removing one solvent from the solvent mixture comprising at least two solvents of the inventive composition, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation means, the diameter of the surface of said separation means being larger than the height of said separation means; the separation means comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica; the solid support, preferably silica, having a particle size ranging from 5 μm to 1000 μm, preferably from 10 μm to 150 μm, more preferably ranging from 30 μm to 100 μm and most preferably ranging from 40 μm to 63 μm; and a mean pore size ranging from 1 to 100 nm; the solid support being suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and the slurry thus obtained being applied to the separation means; then dissolving or suspending the composition after step iia), step iib) or step iic) in a diluting solvent or solvent mixture selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and applying the diluted composition thus obtained onto the separation means (step iii), with the suspending solvent or mixture of suspending solvents being identical with or different to the diluting solvent or the diluting solvent mixture; iv) optionally subjecting the remainder from step iii) to a further distillation.

Another embodiment dealing with a separation column instead of a separation means is defined as follows:

A process for obtaining a quinone preparation comprising the steps: i) removing one solvent from the solvent mixture comprising at least two solvents of the inventive composition, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation column, the separation column comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica; the solid support, preferably silica, having a particle size ranging from 5 μm to 1000 μm, preferably from 10 μm to 150 μm, more preferably ranging from 30 μm to 100 μm and most preferably ranging from 40 μm to 63 μm; and a mean pore size ranging from 1 to 100 nm; the solid support being suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and the slurry thus obtained being applied to the separation column; then dissolving or suspending the composition after step iia), step iib) or step iic) in a diluting solvent or solvent mixture selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane or n-heptane, and applying the diluted composition thus obtained onto the separation column (step iii)), with the suspending solvent or mixture of suspending solvents being different to the diluting solvent or the diluting solvent mixture; iv) optionally subjecting the remainder from step iii) to a further distillation.

However, when suspending solvent and diluting solvent are of the same nature, likewise good results were obtained.

A process for obtaining a quinone preparation comprising the steps: i) removing one solvent from the solvent mixture comprising at least two solvents of the inventive composition, or removing the C-bearing solvent of the composition of the invention; with optionally adding hydrochloric acid prior or during removing one solvent from the solvent mixture or with optionally adding hydrochloric acid prior or during removing the C-bearing solvent; iia) distilling off remaining solvent(s) or iib) degassing the composition or iic) distilling off remaining solvent(s) and degassing the composition; iii) applying the composition of step iia), step iib) or step iic) onto a separation column, the separation column comprising a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA), preferably being silica; the solid support, preferably silica, having a particle size ranging from 5 μm to 1000 μm, preferably from 10 μm to 150 μm, more preferably ranging from 30 μm to 100 μm and most preferably ranging from 40 μm to 63 μm; and a mean pore size ranging from 1 to 100 nm; the solid support being suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane, n-heptane or cyclohexane, and the slurry thus obtained being applied to the separation column; then dissolving or suspending the composition after step iia), step iib) or step iic) in a diluting solvent or solvent mixture selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, preferably in an aliphatic hydrocarbon and most preferably in n-hexane or n-heptane, and applying the diluted composition thus obtained onto the separation column (step iii)), with the suspending solvent or mixture of suspending solvents being identical with the diluting solvent or the diluting solvent mixture; iv) optionally subjecting the remainder from step iii) to a further distillation.

Another crucial feature for obtaining a quinone preparation of the invention is a process where iii) after applying the composition of step iia), iib) or step iic) onto the separation means, the diameter of the surface of said separation means being larger than the height of said separation means or after applying the composition of step iia), iib) or step iic) onto the separation column; iiia) one elutes impurities and by-products with a mixture of a non-polar and a polar solvent having a volumetric ratio ranging from 90:10 to 99:1, preferably from 92:8 to 98:2 and mostly preferred from 94:6 to 97:3; iiib) one elutes the product with a mixture of a non-polar and a polar solvent having a volumetric ratio ranging from 60:40 to 85:15, preferably from 70:30 to 82:18 and mostly preferred from 75:25 to 80:20; iv) optionally one subjects the remainder from step iiib) to a further distillation, preferably to at least one further distillation or, iii) after applying the composition of step iia), iib) or step iic) onto the separation means, the diameter of the surface of said separation means being larger than the height of said separation means or after applying the composition of step iia), iib) or step iic) onto the separation column; iiia) one elutes the product with a mixture of a non-polar and a polar solvent having a volumetric ratio ranging from 60:40 to 85:15, preferably from 70:30 to 82:18 and mostly preferred from 75:25 to 80:20; iiib) one elutes impurities and by-products with a mixture of a non-polar and a polar solvent having a volumetric ratio ranging from 90:10 to 99:1, preferably from 92:8 to 98:2 and mostly preferred from 94:6 to 97:3; iv) optionally one subjects the remainder from step iiia) to a further distillation, preferably to at least one further distillation.

A non-polar solvent as understood within this disclosure is a solvent selected from the group consisting of aliphatic hydrocarbons, aromatic hydrocarbons, halogenated hydrocarbons, ethers. The meaning of each of these solvent groups is as indicated supra.

A polar solvent as defined in this specification is a solvent selected from the group consisting of alcohols, carboxylic acids, esters, ketones, acetals, ketals, nitriles and water. Each solvent group has the meaning as defined supra.

The process of obtaining a quinone preparation of the invention is further specified by the nonpolar solvent being at least one of heptane or cyclohexane, the polar solvent being at least one of isopropyl acetate or ethyl acetate and the mixture of the non-polar solvent and the polar solvent comprising at least one polar solvent and at least one non-polar solvent. As can be seen from the examples 1019 (CN101), 1027 (CN98), 1052 (CN1) with 1053 (CN2), 1056 (CN3), 1057 (CN107) below, quinone preparations having low traces of chloride, organic chlorine and copper ions were obtained with these solvents used.

A further substantial embodiment of the disclosed invention is a quinone preparation, preferably as obtained by one of the previously disclosed process embodiments. Said quinone preparation preferably obtained by the inventive process comprises: A) 90 to 100 w % of quinone C30

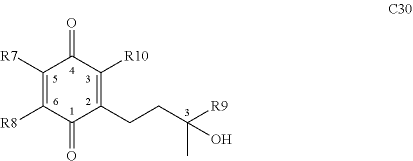

with R7, R8, R10 being H or CH₃; R9 being alkyl, alkenyl, preferably 94 to 100 w % of quinone C30, more preferably 96 to 100 w %, even more preferred >96 to 100 w % and mostly preferred 98 to 100 w %; B) 0,0001 to 9999/1000 ppm of Cu, preferably 0,0001 to 2999/1000 ppm of Cu; C) 0,0001 to 100 ppm of organic chlorine, preferably 4 to 78 ppm; D) minor components with the sum of A) to D) adding up to 100 w %.

Another substantial embodiment of the invention is a quinone preparation preferably obtained by the inventive process as disclosed in at least one of the previous embodiments, comprising: A) 90 to 100 w % of quinone (C30)

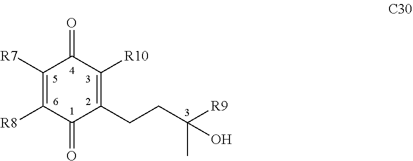

with R7, R8, R10 being H or CH₃; R9 being alkyl, alkenyl, preferably 94 to 100 w % of quinone C30, more preferably 96 to 100 w %, even more preferred >96 to 100 w %, still further preferred 98 to 100 w % and mostly preferred 100 w % minus the amount of components B) to D) as defined below; B) 0,0001 to 9999/1000 ppm of Cu, preferably 0,0001 to 2999/1000 ppm of Cu; C) 0,0001 to 100 ppm of organic chlorine, preferably 4 to 78 ppm; D) minor components with minor components being all chemical entities besides those mentioned under A), B) and C) which at most amount up to 10 w % minus the amount of components B) and C), preferably with minor components being all chemical entities besides those mentioned under A), B) and C) which at most amount up to 6 w % minus the amount of components B) and C), further preferred with minor components being all chemical entities besides those mentioned under A), B) and C) which at most amount up to 4 w % minus the amount of components B) and C), yet further preferred with minor components being all chemical entities besides those mentioned under A), B) and C) which at most amount to a value, which is smaller than 4 w % minus the amount of components B) and C), yet further preferred with minor components being all chemical entities besides those mentioned under A), B) and C) which at most amount up to 2 w % minus the amount of components B) and C) and mostly preferred with minor components being all chemical entities besides those mentioned under A), B) and C) which in a first embodiment at most amount to 300 ppm, in a second embodiment at most amount to 200 ppm, in a third embodiment at most amount to 100 ppm and with the sum of A) to D) adding up to 100 w %.

Said quinone preparation is adapted to satisfy demands of purity and of a trace amount spectrum as required by the feed, the dietary supplement or the pharmaceutical industry. Such preparation hence can be directly delivered to a customer.

A further embodiment is the use of an inventive quinone preparation in animal nutrition or as dietary supplement, or as beverage additive.

The invention will now be further specified by explaining the analytical methods employed, by describing one embodiment of the oxidation of the chroman C1 and one embodiment of the process of obtaining a quinone C30 preparation. Thereafter, the examples as indicated supra will be explained in detail.

Method for assaying the amount of quinone C30 in or from a reaction mixture by means of HPLC

Assays were realized on a Zorbax Eclipse PAH HPLC column (particle size 1.8 mm, 50 mm×4.6 mm) from Agilent® incorporated into an Agilent Series 1100 HPLC. The elution system was solvent A consisting of 0,1 v % of orthophosphoric acid in water, solvent B consisting of acetonitrile. The elution profile was as follows:

TABLE 11

| time [min] | % B | flow ml/min |
|---|---|---|
| 0.0 | 50 | 1.2 |
| 8.0 | 100 | 1.2 |
| 12.0 | 100 | 1.2 |
| 12.1 | 50 | 1.2 |

Injection volume was 5 μl and elution took place at 60° C.

Calibration was realized with an external standard of five substances as indicated by Agilent® the respective concentration of each was:

Substance 1: 0.04 g/L

Substance 2: 0.08 g/L

Substance 3: 0.12 g/L

Substance 4: 0.16 g/L

Substance 5: 0.20 g/L

and giving a calibration straight line when plotting the concentration against the elution time.

A sample as indicated in the examples infra was weighed in a 100 ml volumetric flask and solubilized or diluted in a predefined amount of either acetonitrile or tetrahydrofuran. An aliquot of 5 μl of said solution was injected onto the HPLC column.

The % values as given in the examples infra are area percent values based on the total peak areas obtained in the respective chromatogram. They can be converted into w % values according to the following equations:

w%=(peak area×response factor of analyzed substance)/sample weight

response factor=weight of analyzed substance/area of analyzed substance

### Method for Determining the Amount of Cu Ions

### Sample Preparation

300-400 mg of the sample were weighed, to the nearest 0.1 mg, and digested as follows:
Cracking of the sample with concentrated sulfuric acid conc. (8 ml) at 320° C.
Complete digestion of organic remnants with 7 ml of an acid mixture of nitric acid, perchloric acid and sulfuric acid all concentrated at a volume ratio of 2:1:1 at 160° C.
Evaporation of excess acids
Addition of 50% (v/v) hydrochloric acid to the residue and heating to boiling

After completion of the digestion, the exact volume of the solution obtained was determined by weighing and corrected according to the appropriate density.

The analysis was performed in duplicate. A blank was run in an analogous manner.

### Determination

Copper was determined with the obtained solution as is by inductively coupled plasma-optical emission spectrometry (ICP-OES) using an ICP-OES Agilent 5100 apparatus. The detection wavelength employed was: Cu 324.754 nm and an internal standard of Sc 361.383 nm was used via internal loop. Calibration was realized with an external standard.

### Method for Determining the Amount of Chloride, Viz Chloride Ions in Ppm

### Sample Preparation:

An aliquot of 200 mg of the sample was weighed into a centrifugal tube and supplemented with 10 ml of toluene and 10 ml ultrapure water. After separation of the organic phase, the remaining aqueous phase was used for ion chromatographic analysis. The analysis was performed in duplicate.

A blank was run in an analogous manner.

### Measurement:

Chloride was determined by ion chromatography; detection was carried out by means of a conductivity detector (after suppression of basic conductivity):

### Measurement Parameters:

Apparatus: 850 Professional IC (Metrohm)

Pre-column: Metrosep A Supp 4/5 S-Guard

Column: Metrosep A Supp 5 250×4.0 mm

Eluent: 3.2 mmol Na₂CO₃/1.0 mmol NaHCO₃

Eluent flow: 0.7 ml/min

Suppressor: MSM (Metrohm)

Injection volume: 25 μl

Column temperature: 45° C.

Detector temperature: 40° C.

Calibration range: ß(Cl—)=10 μg/l-200 μg/l

### Method for Determining the Amount of Total Chlorine in Ppm

Total chlorine was determined by microcoulometry using the protocol provided with the apparatus Xplorer®, an elemental combustion analyzer of the company Trace Elemental Instruments.

In particular an aliquot of 10 to 20 mg of the sample to be analyzed was burned in an oxygen/argon atmosphere (furnace temperature: 1050° C.). The resulting hydrochloric acid sample was freed from by-products of the combustion like sulfur, nitrogen oxides and water and transferred into a coulometric titration cell. Within said cell automatic titration of chloride ions takes place with automatically generated silver ions according to the equations:

Ag→Ag⁺+e⁻ (electrolysis)

Ag⁺+Cl⁻→AgCl

Each analysis was performed in duplicate.

### Method of Determining the Amount of Organic Chlorine

The amount of organic chlorine was determined as follows:

Organic chlorine [ppm]=total chlorine [ppm]−chloride [ppm]

Each example has its example number. For the sake of easier retrieval, each example was also allotted a consecutive number CN.

### CN1, EXAMPLE 1052

### Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 144,20 g (312.51 mmol) of α-tocopherol of formula C5 were solubilized in 386,38 g (3.8 mol) of 3-hexanol and added to the reactor. The reaction mixture was maintained at 25° C. while bubbling 40 l/h of air through it for a period of 4,75 h (altogether being an embodiment of the inventive composition). The aqueous phase was removed. The organic phase was washed three times with water at 48° C. and the at least one solvent or the C-bearing solvent of the organic phase removed under reduced pressure. 150,9 g of crude α-tocopherol quinone of formula C33 (MW=446.71 g/mol) corresponding to a yield of 94,1% were obtained.

### CN2, EXAMPLE 1053

Purification of Sample from CN1 by Degassing

148,6 g of crude α-tocopherol quinone of formula C33 were subjected to a reduced pressure of 2,3×10² Pa and a temperature of 110° C. for 155 min, after which 132,8 g of α-tocopherol quinone of formula C33 were obtained. The amount of organic chlorine was 73 ppm, of chloride was 47 ppm and of Cu ions was 70 ppm.

### CN3, EXAMPLE 1056

Further purification of sample from CN2 by application onto a short-plug as separation means

A G3 glass suction filter (volume of 1 l, 12.5 cm inner diameter) was filled with a slurry of silica (particle size 40 to 63 μm) in n-heptane to a height of 6.7 cm giving a volume of 822 ml. 35,5 g of α-tocopherol quinone of formula C33 from CN2 (example 1053) were dissolved in 14,2 g of n-heptane and applied onto the wet silica. Under suction another 1000 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 34,0 g of α-tocopherol quinone of formula C33 (quinone preparation of the invention). The amount of organic chlorine in said quinone preparation was 18 ppm, the amount of chloride <3 ppm and the amount of Cu was <3 ppm.

### CN4, COMPARATIVE EXAMPLE 384

### Reaction of Chroman C1 in the Absence of Catalyst

25 g (58.04 mmol) of α-tocopherol of formula C5 were solubilized in 225 g of dimethylformamide and the reaction mixture supplemented with 30 l/h of air for 6 h at room temperature. Thereafter 0,8 g (5.8 mmol) of potassium bicarbonate were added with stirring and 30 l/h of air was added for another 24 h. Potassium bicarbonate was filtered off and a sample taken for HPLC analysis. No quinone C30 could be detected.

### CN5, COMPARATIVE EXAMPLE 389

### Reaction of Chroman C1 in the Absence of Catalyst

32 g (74.29 mmol) of α-tocopherol of formula C5 were solubilized in 92,27 g of n-hexanol and the reaction mixture supplemented with 30 l/h of air for 6 h at room temperature. A sample was taken for HPLC analysis. No quinone C30 could be detected.

### CN6, COMPARATIVE EXAMPLE 1023

Reaction of Chroman C1 without Actively Moving a Gaseous Compound Containing Oxygen Through the Reactor

55,0 g (120 mmol) of α-tocopherol of formula C3 or C5 were solubilized in 550 ml solvent. 5,12 g (30.0 mmol) of CuCl₂×2 H₂O, CAS no: 10125-13-0 were added. The mixture was left standing under air for 8 h. Afterwards in the case of the solvent methanol 200 ml of cyclohexane and 100 ml of water were added, in the case of the solvent n-hexanol 200 ml of water were added. The phases were separated. The organic phase was washed with water and the respective yield was determined from the organic phase by HPLC-w % as described in the Table 12.

TABLE 12

| | R,R,R-α-tocopherol | | rac-α-tocopherol | |
|---|---|---|---|---|
| | quinone of | | quinone of | |
| | formula C33 | | formula C32 | |
| | Yield [%] | | Yield [%] | |
| Solvent | Methanol | n-Hexanol | Methanol | n-Hexanol |
| 0.25 eq CuCl₂ × 2 H₂O | 5.2 | 2.7 | 3.5 | 2.4 |

### CN7, COMPARATIVE EXAMPLE 1004

Reaction of Chroman C1 without Actively Moving a Gaseous Compound Containing Oxygen Through the Reactor

1,0 g (2.32 mmol) of α-tocopherol of formula C3 or C5 was respectively solubilized in 10 ml solvent and each mixture placed in a distinct 100 ml Erlenmeyer flask. 1,0 g, (37.2 mmol) of CuCl₂, CAS no: 7447-39-4 was added to each mixture. Each flask was placed on a shaker set at a speed of 40 rpm at room temperature and shaken for 8 h or 16 h respectively. After 8 h or 16 h the reaction mixture was filtered over 1,5 g silica to remove CuCl₂. The silica was washed with the solvent used for the reaction. The yields determined by HPLC-w % in the solution after filtration are described in Table 13.

TABLE 13

| | R,R,R-α-tocopherol quinone of | | rac-α-tocopherol quinone of | |
|---|---|---|---|---|
| | formula C33 | | formula C32 | |
| | Yield [%] | | Yield [%] | |
| Solvent | Methanol | n-Hexanol | Methanol | n-Hexanol |
| 8 h | 14.6 | 17.0 | 13.2 | 10.1 |
| 16 h | 31.7 | 17.6 | 32.0 | 18.6 |

### CN8, COMPARATIVE EXAMPLE 903

Reaction of Chroman C1 without Actively Moving a Gaseous Compound Containing Oxygen Through the Reactor

5,0 g (11.00 mmol) of α-tocopherol of formula C5 were solubilized in 39,5 g of methanol and 5,0 g, (37.19 mmol) of CuCl₂, CAS no: 7447-39-4 were added. The whole was stirred for 48 h at room temperature. 20 ml of cyclohexane and 25 ml of bi-distilled water were added. The organic phase was washed two times with 25 ml of bi-distilled water and the solvent of the unified organic phases was removed under reduced pressure. 5,8 g of a crude product were obtained containing 4,59 w % of quinone of formula C33. This corresponds to a yield of 5,4% as determined by HPLC.

### CN9, COMPARATIVE EXAMPLE 1015

Reaction of Chroman C1 without Actively Moving a Gaseous Compound Containing Oxygen Through the Reactor

55,0 g (94,0%, 120 mmol) of rac-α-tocopherol of formula C3 were solubilized in 550 ml of the respective solvent. 5,12 g (30.0 mmol) of CuCl₂×2 H₂O, CAS no: 10125-13-0 were added respectively. Each mixture was stirred with a speed of 100 rpm. After 8 h in the case of methanol as solvent, 200 ml of cyclohexane and 100 ml of water, in the case of n-hexanol as solvent, 200 ml of water were added to each mixture. The phases separated. The organic phase obtained from each mixture was washed with water and the yields were determined in each organic phase by HPLC-w % as shown in the Table 14.

TABLE 14

| | rac-α-tocopherol quinone of formula C32 | |
|---|---|---|
| | Yield [%] | |
| Solvent | Methanol | n-Hexanol |
| 0.25 eq CuCl₂ × 2 H₂O | 9.5 | 9.9 |

### CN10, EXAMPLE 968

### Use of an Appropriate Amount of Catalyst, Semi-Batchwise Synthesis of α-Tocopherol Quinone of Formula C32

1,69 g (9.91 mmol) of CuCl₂×2 H₂O, CAS no: 10125-13-0 were solubilized in 15 g (0.83 mol) of water and placed in the reactor together with 83 g of n-hexanol. A solution of 44,90 g (98.93 mmol) of α-tocopherol of formula C3 in 39,9 g of n-hexanol was added dropwise at 25° C. during 4 h. The mixture was stirred for another 8 h. During the whole reaction air at a rate of 12 to 14 l/h was bubbled through the reaction mixture while stirring with 1200 rpm. After termination of the reaction 54 ml of bi-distilled water were added to the mixture and the phases were separated. The organic phase was washed twice with 54 ml of bi-distilled water. A sample of the organic phase was taken and revealed a yield of 92,8% of α-tocopherol quinone of formula C32 as determined by HPLC.

### CN11, EXAMPLE 952

### Use of an Appropriate Amount of Catalyst, Semi-Batchwise Synthesis of α-Tocopherol Quinone of Formula C32

4,22 g (24.75 mmol) of CuCl₂×2 H₂O, CAS no: 10125-13-0 and 4,19 g (98.85 mmol) of LiCl, CAS no: 7447-41-8 were dissolved in 35,7 g of bi-distilled water, supplemented with 33 g of n-hexanol and placed in the reactor. A solution of 90 g n-hexanol containing 42,15 g (98.83 mmol) of α-tocopherol of formula C3 was added dropwise at room temperature into the reactor during a time span of 2 h with simultaneously injecting into reaction mixture air with a rate of 12 to 14 l/h. The reaction mixture was stirred for another 6 h at 1000 rpm while air was further bubbled through the mixture. The organic phase was separated and washed three times with 30 ml of bi-distilled water (35° C.). A sample of this purified organic phase was determined by HPLC-w % to show a yield of 92,7% of quinone of formula C32.

### CN12, EXAMPLE 985

### Use of an Appropriate Amount of Catalyst, Semi-Batchwise Synthesis of α-Tocopherol Quinone of Formula C32

13,32 g (78.13 mmol) of CuCl₂×2 H₂O, CAS no: 10125-13-0 were solubilized in 28,15 g of water and placed in the reactor. A solution of 134,6 g (312.51 mmol) of α-tocopherol of formula C3 in 388,3 g of n-hexanol was added dropwise at 25° C. during 2 h. The mixture was further stirred for 5 h. During the whole reaction air at a rate of 40 l/h was bubbled through the reaction mixture and the reaction mixture while stirred at 1000 rpm. After termination of the reaction the aqueous phase was separated. A sample of the upper organic phase was taken and revealed a yield of 96% of α-tocopherol quinone of formula C32 as determined by HPLC-w %.

### CN13, EXAMPLE 988

### Use of an Appropriate Amount of Catalyst, Semi-Batchwise Synthesis of α-Tocopherol Quinone of Formula C32

13,32 g (78.13 mmol) of CuCl₂×2 H₂O, CAS no: 10125-13-0 were solubilized in 28,15 g of water and placed in the reactor together with 87,46 g of n-hexanol. A solution of 134,60 g (312.51 mmol) of α-tocopherol of formula C3 in 298,86 g of n-hexanol was added dropwise at 25° C. during 2 h and the mixture was further stirred for 4,5 h. During the whole time air at a rate of 40 l/h was bubbled through the reaction mixture while stirring at 1000 rpm. After termination of the reaction the aqueous phase was separated. A sample of the upper organic phase was taken and revealed a yield of 97% of α-tocopherol quinone of formula C32 as determined by HPLC.

### CN14, EXAMPLE 905

### Use of an Appropriate Amount of Catalyst, Semi-Batchwise Synthesis of α-Tocopherol Quinone of Formula C33

13,32 g (78.1 mmol) of CuCl₂×2 H₂O, CAS no: 10125-13-0 were dissolved in 28,2 g (1.6 mol) of water and placed in the reactor. 143,2 g (312.5 mmol) of α-tocopherol of formula C5 were solubilized in 386,4 g (3.8 mol) of n-hexanol and added to the reactor. The reaction mixture was stirred at 1000 rpm at 25° C. while bubbling 40 l/h of air through the mixture for 6 h. The aqueous phase was separated and the organic phase was washed three times with 170 ml water at 25° C. The solvent was removed at 100° C./8×102 Pa and the product further degassed at 100° C./2×102 Pa yielding 100% of quinone C33 as determined by HPLC-w %. By the methods indicated supra, the amount of organic chlorine was determined to be 77 ppm, the amount of chloride was determined to be 21 ppm and the amount of Cu ions was determined to be 13 ppm.

### CN15, EXAMPLE 1052, CF. CN1

### Use of an Appropriate Amount of Catalyst, Batchwise Synthesis of α-Tocopherol Quinone of Formula C32

### CN16, EXAMPLE 1086

### Use of an Appropriate Amount of Catalyst, Semi-Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,38 g (3.78 mol) of n-hexanol and added to the reactor. The reaction mixture was stirred at 1000 rpm at 25° C. while bubbling 80 l/h of air through it for a period of 4 h. The aqueous phase was removed. The organic phase was washed three times with 170 ml water at 40° C. and a sample taken from the washed organic phase revealed a yield of 95% of quinone C33 as determined by HPLC-w %.

### CN17, EXAMPLE 977

### Use of an Appropriate Amount of Catalyst, Semi-Batchwise Synthesis of α-Tocopherol Quinone of Formula C32

40,07 g (235.04 mmol) of CuCl₂×2 H₂O, CAS no: 10125-13-0 were placed in the reactor and solubilized in 84.6 g of water. A solution of 101,23 g (235.03 mmol) of α-tocopherol of formula C3 in 291,9 g of n-hexanol was added dropwise at 25° C. during a time span of 2 hours into the reactor while stirring at 1200 rpm and injecting air into the reaction mixture with a rate of 30 l/h. Stirring and air injection was continued for an additional hour after which a sample of the upper viz. organic phase was taken for HPLC analysis indicating a yield of 100% of quinone of the formula C32.

### CN18, EXAMPLE 979

### Use of an Appropriate Amount of Catalyst, Semi-Batchwise Synthesis of α-Tocopherol Quinone of Formula C32

16,87 g (99.0 mmol) of CuCl₂×2 H₂O, CAS no: 10125-13-0 were dissolved in 36,0 g (2.0 mol) of water and placed in the reactor. 83,0 g of n-hexanol were added to the reactor. 42,6 g (98.9 mmol) of rac-α-tocopherol C3 were dissolved in 39,9 g n-hexanol and added to the reactor within 4 h followed by 1 h of stirring (1200 rpm). Through the whole time the reaction mixture was maintained at 25° C. while bubbling 12-14 l/h of air through it. The aqueous phase was removed and the organic phase was washed three times with 54 ml water. The yield of quinone C32 in the organic phase was determined to be 95,5% by HPLC-w %.

### CN19, EXAMPLE 977 CF. CN17

### High Yield in Short Reaction Times, Batchwise Synthesis of α-Tocopherol Quinone of Formula C32

### CN20, EXAMPLE 1052 CF. CN1

### High Yield in Short Reaction Times, Batchwise Synthesis of α-Tocopherol Quinone of Formula C33

### CN21, EXAMPLE 1021

### High Yields in Short Reaction Times, Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,42 g (3.78 mol) of n-hexanol and added to the reactor. The reaction mixture was maintained at 25° C. with stirring at 1000 rpm while bubbling 40 l/h of air through it for a period of 4,75 h. A sample of the organic phase was taken and revealed 99% of α-tocopherol quinone of formula C33 as determined by HPLC-w %.

### CN22, EXAMPLE 1060

### High Yields in Short Reaction Times, Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,36 g (3.78 mol) of n-hexanol and added into the reactor. The reaction mixture was maintained at 25° C. under stirring at 1000 rpm while bubbling 40 l/h of air through it for 4,75 h. The aqueous phase was removed. The organic phase was washed three times with water at 48° C. and the solvent removed at 90° C. at 2×102 Pa. A sample of the residue was taken and revealed a yield of 100% of α-tocopherol quinone of formula C33 as determined by HPLC-w %.

### CN23, EXAMPLE 946

### High Yields in Short Reaction Times, Batchwise Synthesis of α-Tocopherol Quinone of Formula C32

4,22 g (24.6 mmol) of CuCl₂×2 H₂O, CAS no: 10125-13-0 and 10,06 g (49.48 mmol) of MgCl₂×6 H₂O were solubilized in 35.7 g of water and placed in the reactor. A solution of 42,6 g (98,93) mmol) of α-tocopherol of formula C3 in 122,9 g of n-hexanol was also placed in the reactor. Thereafter air at a rate of 12 to 14 l/h was bubbled through the mixture while said mixture being stirred with air intake for 5 h at 23° C. After termination of the reaction the phases were separated and the organic phase was washed 3 times with 30 ml of water at 35° C. A sample of the organic phase was taken and revealed a yield of 94,9% of α-tocopherol quinone of formula C32 as determined by HPLC-w %.

### CN24, EXAMPLE 1054

### High Yields in Short Reaction Times, Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,38 g (3.33 mol) of 3-heptanol and added to the reactor. The reaction mixture was maintained at 25° C. with stirring at 1000 rpm, while bubbling 40 l/h of air through it for a period of 5 h. The aqueous phase was removed. The organic phase was washed three times with 170 ml of water at 46° C. and the at least one solvent or the C-bearing solvent of the organic phase removed at 90° C. during 90 min under reduced pressure. A sample was taken and revealed a yield of 95,2% of α-tocopherol quinone of formula C33 as determined by HPLC-w %. The amount of organic chlorine was analyzed by the methods indicated supra to be 60 ppm, the amount of chloride was 26 ppm and the amount of Cu was 12 ppm.

### CN25, EXAMPLE 1032

### High Yields in Short Reaction Times, Semi-Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor, which was thereafter supplemented with 87,5 g (856.42 mmol) of n-hexanol. The reaction mixture was maintained at 40° C. under stirring at 1000 rpm while bubbling 40l/h of air through it. 134,60 g (312.51 mmol) of α-tocopherol of formula C5 were solubilized in 298,87 g (2.93 mol) of n-hexanol and added dropwise into the reactor during 4 h while stirring and bubbling. After a further hour of reaction, the aqueous phase was removed. The organic phase was washed three times with water and the solvent removed at 90° C. and 2×102 Pa. A sample of the residue was taken and revealed a yield of 99% of α-tocopherol quinone of formula C33 as determined by HPLC-w %. With the methods indicated supra, the amount of organic chlorine was determined to be 149 ppm, the amount of chloride to be 21 ppm and the amount of Cu ions to be 31 ppm.

### CN26, EXAMPLE 877

### High Yields in Short Reaction Times, Batchwise Synthesis of α-Tocopherol Quinone of Formula C33

13,32 g (78.1 mmol) of CuCl₂×2 H₂O, CAS no: 10125-13-0 were dissolved in 28,2 g (1.6 mol) of water and placed in the reactor. 143,2 g (312.5 mmol) of α-tocopherol of formula C5 were solubilized in 386,4 g (3.8 mol) of n-hexanol and added to the reactor. The reaction mixture was stirred at 1000 rpm at 15° C. while bubbling 40 l/h of air through the mixture for 6 h. The aqueous phase was separated, and the organic phase was washed three times with 170 ml water at 45-50° C. The solvent was removed at 100° C./10×10² Pa and the product further degassed at 100° C./1×10² Pa yielding 99,1% of quinone C33 as determined by HPLC-w %. By the methods indicated supra, the amount of organic chlorine was determined to be 27 ppm, the amount of chloride was determined to be 9 ppm and the amount of Cu ions was determined to be 5 ppm.

### CN27, EXAMPLE 905 CF. CN14

### High Yields in Short Reaction Times, Batchwise Synthesis of α-Tocopherol Quinone of Formula C33

### CN28, EXAMPLE 935

### High Yields in Short Reaction Times, Semi-Batchwise Synthesis of α-Tocopherol Quinone of Formula C32

5,8 g (25.9 mmol) of CuBr₂, CAS no: 7789-45-9 were solubilized in 9.4 g of water and placed in the reactor together with 38,8 g of 2-ethyl-1-hexanol. A solution of 42,61 g (98.99 mmol) of α-tocopherol of formula C3 in 90,6 g of 2-ethyl-1-hexanol was added dropwise at 50° C. during 2 h. The mixture was further stirred for 5 h at 50° C. During the whole reaction air at a rate of 12 to 14l/h was bubbled through the reaction mixture while stirring at 1000 rpm. After termination of the reaction, the phases were separated, and the organic phase was washed 3 times with 30 ml of bi-distilled water. A sample of the organic phase was taken and revealed 34% of α-tocopherol quinone of formula C32 as determined by HPLC-w %.

### CN29, EXAMPLE 942

### High Yields in Short Reaction Times, Semi-Batchwise Synthesis of α-Tocopherol Quinone of Formula C32

4,22 g (24.6 mmol) of CuCl₂×2 H₂O, CAS no: 10125-13-0 and 10,06 g (49.48 mmol) of MgCl₂×6 H₂O were solubilized in 35.7 g of water and placed in the reactor together with 34,6 g of n-hexanol. A solution of 42,6 g (98.93 mmol) of α-tocopherol of formula C3 in 88,3 g of n-hexanol was added dropwise at 23° C. during 2 h followed by stirring for another 5 h. During the whole reaction air at a rate of 12 to 14 l/h was bubbled through the reaction mixture while stirring at 1000 rpm. After termination of the reaction, the phases were separated and the organic phase was washed 3 times with 30 ml of bi-distilled water (35° C.). A sample of the organic phase was taken and revealed a yield 97,5% of α-tocopherol quinone of formula C32 as determined by HPLC-w %.

### CN30, EXAMPLE 952 CF. CN11

### High Yields in Short Reaction Times, Semi-Batchwise Synthesis of α-Tocopherol Quinone of Formula C32

### CN31, EXAMPLE 976

### High Yields in Short Reaction Times, Batchwise Synthesis of α-Tocopherol Quinone of Formula C32

1,69 g (9.91 mmol) of CuCl₂×2 H₂O, CAS no: 10125-13-0 were solubilized in 15 g of water and placed in the reactor. A solution of 42,6 g (98.93 mmol) of α-tocopherol of formula C3 in 122,3 g of n-hexanol was likewise placed in the reactor. The reactor was warmed to 25° C. and air was bubbled through the reaction mixture at a rate of 12 to 14l/h for 10 h while stirring at 1200 rpm. 54 ml of bi-distilled water were added followed by stirring and phase separation. The organic phase was washed two times with 54 ml of bi-distilled water. A sample of the organic phase was taken and revealed a yield of 92,5% of α-tocopherol quinone of formula C32 as determined by HPLC-w %.

### CN32, EXAMPLE 941

### Further Metal Compound in Addition to CuCl₂, Batchwise Synthesis of α-Tocopherol Quinone of Formula C32

4,2 g (24.64 mmol) of CuCl₂×2 H₂O, CAS no: 10125-13-0 and 4,19 g (98,85 mmol) of LiCl, CAS no: 7447-41-8 were dissolved in 35,65 g (1.98 mol) of water and placed in the reactor. 42,6 g (98.93 mmol) of α-tocopherol of formula C3 were solubilized in 122,92 g (1.20 mol) of n-hexanol and added into the reactor. The reaction mixture was maintained at 25° C. under stirring at 1000 rpm while bubbling 12-14 l/h of air through it during 5 h. The aqueous phase was removed. The organic phase was washed three times with 30 ml of water. A sample of the organic phase was taken and revealed 94,6% of α-tocopherol quinone of formula C32 as determined by HPLC-w %.

### CN33, EXAMPLE 946 CF. CN23

### Further Metal Compound in Addition to CuCl₂, Batchwise Synthesis of α-Tocopherol Quinone of Formula C32

### CN34, EXAMPLE 390

Amount of Metal Compound Used with Respect to Chroman C3, Semi-Batchwise Synthesis of α-Tocopherol Quinone of Formula C32

4,02 g (23.58 mmol) of CuCl₂×2 H₂O, CAS no: 10125-13-0 and 3,99 g (94,13 mmol) of LiCl, CAS no: 7447-41-8 were dissolved in 84,65 g (4.69 mol) of water and placed in the reactor. 101,23 g (235.03 mmol) of α-tocopherol of formula C3 were solubilized in 291,9 g (2.86 mol) of n-hexanol and added into the reactor during 2 h 15 min followed by stirring for 10,3 h. During the whole reaction the reaction mixture was maintained at 22 to 25° C. with stirring at 1000 rpm while bubbling 30 l/h of air through the reaction mixture. After addition of water and phase separation a sample of the organic phase revealed a yield of 87,2% of α-tocopherol quinone of formula C32 as determined by HPLC-w %.

### CN35, EXAMPLE 946, CF. CN23

Amount of Metal Compound Used with Respect to Chroman C3, Batchwise Synthesis of α-Tocopherol Quinone of Formula C32

### CN36, EXAMPLE 952, CF. CN11

Amount of Metal Compound Used with Respect to Chroman C3, Semi-Batchwise Synthesis of α-Tocopherol Quinone of Formula C32

### CN37, EXAMPLE 960

### Concentration of Copper Catalyst in One of the at Least Two Solvents of the Solvent Mixture, Semi-Batchwise Synthesis of α-Tocopherol Quinone of Formula C32

4,22 g (24.8 mmol) of CuCl₂×2 H₂O, CAS no: 10125-13-0 were dissolved in 6,3 g (350.0 mmol) of water and placed in the reactor. 27,9 g (273.0 mmol) of n-hexanol were added to the reactor. 44,9 g (98.9 mmol) of α-tocopherol of formula C3 were solubilized in 95,1 g (0.9 mol) of n-hexanol and added dropwise to the reactor over a period of 4 h followed by further stirring for 10 h. During the whole time the reaction mixture was stirred at 1000 rpm at 25° C. and 12-14 l/h of air were bubbled through the mixture. 54 ml of water were added to the reactor and the aqueous phase was separated. The organic phase was washed twice with 54 ml water yielding 87,2% of quinone C32 as determined in the organic phase by HPLC-w %.

### CN38, EXAMPLE 974

### Concentration of Copper Catalyst in One of the at Least Two Solvents of the Solvent Mixture, Semi-Batchwise Synthesis of α-Tocopherol Quinone of Formula C32

3,37 g (19.8 mmol) of CuCl₂×2 H₂O, CAS no: 10125-13-0 were dissolved in 33,0 g (1.8 mol) of water and placed in the reactor. 83,0 g (0.8 mol) of n-hexanol were added to the reactor. 44,9 g (98.9 mmol) of α-tocopherol of formula C3 were solubilized in 39,9 g (0.4 mol) of n-hexanol and added dropwise to the reactor over a period of 4 h followed by further stirring for 5 h. During the whole time the reaction mixture was stirred at 1200 rpm at 25° C. and 12-14 l/h of air were bubbled through the mixture. The aqueous phase was separated, and the organic phase was washed three times with 54 ml water yielding 89,5% of quinone C32 as determined in the organic phase by HPLC-w %.

### CN39, EXAMPLE 958

### Concentration of Copper Catalyst in One of the at Least Two Solvents of the Solvent Mixture, Batchwise Synthesis of α-Tocopherol Quinone of Formula C32

4,22 g (24.8 mmol) of CuCl₂×2 H₂O, CAS no: 10125-13-0 were dissolved in 6,3 g (350.0 mmol) of water and placed in the reactor. 44,9 g (98.9 mmol) of α-tocopherol of formula C3 were solubilized in 123,0 g (1.2 mol) of n-hexanol and added to the reactor. The reaction mixture was stirred at 1000 rpm at 25° C. while bubbling 12-14 l/h of air through the mixture for 18 h. 54 ml of water were added, and the aqueous phase was separated. The organic phase was washed twice with 54 ml water yielding 91,8% of quinone C32 as determined by HPLC-w %.

### CN40, EXAMPLE 952, CF. CN11

### Concentration of Copper Catalyst in One of the at Least Two Solvents of the Solvent Mixture, Semi-Batchwise Synthesis of α-Tocopherol Quinone of Formula C32

### CN41, EXAMPLE 971

### Concentration of Copper Catalyst in One of the at Least Two Solvents of the Solvent Mixture, Semi-Batchwise Synthesis of α-Tocopherol Quinone of Formula C32

3,37 g (19.8 mmol) of CuCl₂×2 H₂O, CAS no: 10125-13-0 were dissolved in 8,0 g (444.4 mmol) of water and placed in the reactor. 83 g (0.81 mol) of n-hexanol were added to the reactor. 44,9 g (98.9 mmol) of α-tocopherol of formula C3 were solubilized in 39,9 g (0.4 mol) of n-hexanol and added dropwise to the reactor over a period of 4 h followed by further stirring for 12 h. During the whole time the reaction mixture was stirred at 1200 rpm at 25° C. and 12-14 l/h of air were bubbled through the mixture. 54 ml of water were added to the reactor and the aqueous phase was separated. The organic phase was washed twice with 54 ml water yielding 93,5% of quinone C32 as determined by HPLC-w %.

### CN42, EXAMPLE 872

### Concentration of Chroman C1 in One of the at Least Two Solvents of the Solvent Mixture, Batchwise Synthesis of α-Tocopherol Quinone of Formula C32

13,32 g (78.1 mmol) of CuCl₂×2 H₂O, CAS no: 10125-13-0 were dissolved in 28,2 g (1.6 mol) of water and placed in the reactor. 142,6 g (312.5 mmol) of α-tocopherol of formula C3 were solubilized in 285,2 g (2.8 mol) of n-hexanol and added to the reactor. The reaction mixture was stirred at 1000 rpm at 25° C. while bubbling 40 l/h of air through the mixture for 5 h. The mixture was washed three times with 170 ml water at 45° C. yielding 97,5% of quinone C32 as determined in the organic phase by HPLC-w %.

### CN43, EXAMPLE 1052, CF. CN1

### Concentration of Chroman C1 in One of the at Least Two Solvents of the Solvent Mixture, Batchwise Synthesis of α-Tocopherol Quinone of Formula C32

### CN44, EXAMPLE 875

### Concentration of Chroman C1 in One of the at Least Two Solvents of the Solvent Mixture, Batchwise Synthesis of α-Tocopherol Quinone of Formula C32

13,32 g (78.1 mmol) of CuCl₂×2 H₂O, CAS no: 10125-13-0 were dissolved in 28,2 g (1.6 mol) of water and placed in the reactor. 142,6 g (312.5 mmol) of α-tocopherol of formula C3 were solubilized in 142,6 g (1.4 mol) of n-hexanol and added to the reactor. The reaction mixture was stirred at 1000 rpm at 25° C. while bubbling 40 l/h of air through the mixture for 5,5 h. The mixture was washed three times with 170 ml water at 45° C. water yielding 91,1% of quinone C32 as determined in the organic phase by HPLC-w %.

### CN45, EXAMPLE 403

### Weight Ratio of Organic Solvent to Water, Batchwise Synthesis of α-Tocopherol Quinone of Formula C33

22,76 g (133.7 mmol) of CuCl₂×2 H₂O, CAS no: 10125-13-0 were dissolved in 47,9 g (2.7 mol) of water and placed in the reactor. 57,2 g (89.5 mmol)R,R,R-α-tocopherol C5 were dissolved in 165,0 g n-hexanol and added to the reactor. The reaction mixture was maintained at 25° C. under stirring (750 rpm) while bubbling 30 l/h of air through it for 7 h. The phases were separated and the aqueous phase was removed. The organic phase was washed three times with 100 ml water and the solvent removed at 85° C./3,5×10² Pa. A sample of the residue was taken and revealed a yield of 97,9% of α-tocopherol quinone of formula C33 as determined by HPLC.

### CN46, EXAMPLE 872, CF. CN42

### Weight Ratio of Organic Solvent to Water, Batchwise Synthesis of α-Tocopherol Quinone of Formula C32

### CN47, EXAMPLE 405

### Weight Ratio of Organic Solvent to Water, Batchwise Synthesis of α-Tocopherol Quinone of Formula C33

11,93 g (70.0 mmol) of CuCl₂×2 H₂O, CAS no: 10125-13-0 were dissolved in 25,1 g (1.4 mol) of water and placed in the reactor. 30,0 g (46.5 mmol) of α-tocopherol of formula C5 were solubilized in 173,0 g (1.7 mol) of n-hexanol and added to the reactor. The reaction mixture was stirred at 850 rpm at 35-40° C. while bubbling 30 l/h of air through the mixture for 6 h. The aqueous phase was separated, the organic phase was washed three times with 100 ml water and the solvent was removed at 85° C. under reduced pressure yielding 96,2% of quinone C33 as determined by HPLC-w %.

### CN48, EXAMPLE 941, CF. CN32

### Weight Ratio of Organic Solvent to Water, Batchwise Synthesis of α-Tocopherol Quinone of Formula C32

### CN49, EXAMPLE 1052, CF. CN1

### Weight Ratio of Organic Solvent to Water, Batchwise Synthesis of α-Tocopherol Quinone of Formula C33

### CN50, EXAMPLE 971, CF. CN41

### Weight Ratio of Organic Solvent to Water, Semi-Batchwise Synthesis of α-Tocopherol Quinone of Formula C32

### CN51, EXAMPLE 968, CF. CN10

### Weight Ratio of Organic Solvent to Water, Semi-Batchwise Synthesis of α-Tocopherol Quinone of Formula C32

### CN52, EXAMPLE 952, CF. CN11

### Weight Ratio of Organic Solvent to Water, Batchwise Synthesis of α-Tocopherol Quinone of Formula C32

### CN53, EXAMPLE 958, CF. 39

### Weight Ratio of Organic Solvent to Water, Batchwise Synthesis of α-Tocopherol Quinone of Formula C32

### CN54, EXAMPLE 875, CF. CN44

### Weight Ratio of Organic Solvent to Water, Batchwise Synthesis of α-Tocopherol Quinone of Formula C32

### CN55, EXAMPLE 974, CF. CN38

### Weight Ratio of Organic Solvent to Water, Semi-Batchwise Synthesis of α-Tocopherol Quinone of Formula C32

### CN56, EXAMPLE 390, CF. CN34

### Weight Ratio of Organic Solvent to Water, Batchwise Synthesis of α-Tocopherol Quinone of Formula C32

### CN57, EXAMPLE 960, CF. CN37

### Weight Ratio of Organic Solvent to Water, Semi-Batchwise Synthesis of α-Tocopherol Quinone of Formula C32

### CN58, EXAMPLE 905, CF. CN14

### Short Reaction Time, Batchwise Synthesis of α-Tocopherol Quinone of Formula C33

### CN59, EXAMPLE 1032, CF. CN25

### Short Reaction Time, Semi-Batchwise Synthesis of α-Tocopherol Quinone of Formula C33

### CN60, EXAMPLE 879

### Short Reaction Time, Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,6 g (312.5 mmol) of α-tocopherol of formula C5 were solubilized in 386,38 g (3.0 mol) of 2-octanol and added to the reactor. The reaction mixture was stirred at 1000 rpm at 25° C. while bubbling 80 l/h of air through the mixture for 6 h. The aqueous phase was separated, and the organic phase was washed three times with 170 ml water at 45° C. The solvent was removed at 130° C./10×10² Pa and the product further degassed at 130° C./1,3×10² Pa yielding 98,8% of quinone C33 as determined by HPLC-w %. By the methods indicated supra, the amount of organic chlorine was determined to be 61 ppm, the amount of chloride was determined to be 9 ppm and the amount of Cu ions was determined to be 11 ppm.

### CN61, EXAMPLE 1021, CF. CN21

### Short Reaction Time, Batchwise Synthesis of α-Tocopherol Quinone of Formula C33

### CN62, EXAMPLE 1074

### Short Reaction Time, Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,38 g (3.33 mol) of n-hexanol and added to the reactor. The reaction mixture was stirred at 1000 rpm at 25° C. while bubbling 40 l/h of air through it for a period of 6 h. The aqueous phase was removed. The organic phase was washed three times with 170 g bi-distilled water at 44-49° C. and a sample taken from the washed and slightly concentrated organic phase revealed a yield of 98% of quinone C33 as determined by HPLC-w %.

### CN63, EXAMPLE 941, CF. CN32

### Short Reaction Time, Batchwise Synthesis of α-Tocopherol Quinone of Formula C32

### CN64, EXAMPLE 877, CF. CN26

### Short Reaction Time, Batchwise Synthesis of α-Tocopherol Quinone of Formula C33

### CN65, EXAMPLE 1054, CF. CN24

### Short Reaction Time, Batchwise Synthesis of α-Tocopherol Quinone of Formula C33

### CN66, EXAMPLE 1052, CF. CN1

### Short Reaction Time, Batchwise Synthesis of α-Tocopherol Quinone of Formula C33

### CN67, EXAMPLE 1086, CF. CN16

### Short Reaction Time, Batchwise Synthesis of α-Tocopherol Quinone of Formula C33

### CN68 EXAMPLE 1024

### Moderate Temperature, Batchwise Synthesis of α-Tocopherol Quinone of Formula C33

9,99 g (58.60 mmol) of CuCl₂×2 H₂O, CAS no: 10125-13-0 were dissolved in 21,11 g (1.17 mol) of water and placed in the reactor. 100,95 g (234.38 mmol) of α-tocopherol of formula C5 were solubilized in 289,77 g (2.84 mol) of n-hexanol and added to the reactor. The reaction mixture was stirred at 1000 rpm at 10° C. while bubbling 30 l/h of air through it for a period of 9 h. The aqueous phase was removed. The organic phase was washed three times with 128 ml water at 40-45° C. and a sample taken from the washed organic phase revealed a yield of 93,6% of quinone C33 as determined by HPLC-w %. The at least one solvent or the C-bearing solvent of the organic phase mainly containing n-hexanol was removed during 45 min under reduced pressure at 90° C. By the methods indicated supra, the amount of organic chlorine was determined to be 100 ppm, the amount of chloride was determined to be 100 ppm and the amount of Cu ions was determined to be 105 ppm.

### CN69, EXAMPLE 877, CF. CN26

### Moderate Temperature, Semi-Batchwise Synthesis of α-Tocopherol Quinone of Formula C33

### CN70, EXAMPLE 883

### Moderate Temperature, Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,38 g (4.4 mol) of n-pentanol and added to the reactor. The reaction mixture was stirred at 1000 rpm at 15° C. while bubbling 40 l/h of air through it for a period of 7 h. The aqueous phase was separated, and the organic phase was washed three times with 170 ml of water at 20-41° C. The solvent was removed at 100° C./10×102 Pa and the product further degassed at 100° C./2,4×102 Pa yielding 93,4% of quinone C33 as determined by HPLC-w %. By the methods indicated supra, the amount of organic chlorine was determined to be 30 ppm, the amount of chloride was determined to be 480 ppm and the amount of Cu ions was determined to be 630 ppm.

### CN71, EXAMPLE 941, CF. CN32

### Moderate Temperature, Batchwise Synthesis of α-Tocopherol Quinone of Formula C32

### CN72, EXAMPLE 942, CF. CN29

### Moderate Temperature, Semi-Batchwise Synthesis of α-Tocopherol Quinone of Formula C32

### CN73, EXAMPLE 1060, CF. CN22

### Moderate Temperature, Semi-Batchwise Synthesis of α-Tocopherol Quinone of Formula C33

### CN74, EXAMPLE 905, CF. CN14

### Moderate Temperature, Semi-Batchwise Synthesis of α-Tocopherol Quinone of Formula C33

### CN75, EXAMPLE 988, CF. CN13

### Moderate Temperature, Semi-Batchwise Synthesis of α-Tocopherol Quinone of Formula C32

### CN76, EXAMPLE 894

### Moderate Temperature, Semi-Batchwise Synthesis of α-Tocopherol Quinone of Formula C33

13,32 g (78.1 mmol) of CuCl₂×2 H₂O, CAS no: 10125-13-0 were dissolved in 28,2 g (1.6 mol) of water and placed in the reactor. 143,2 g (312.5 mmol) of α-tocopherol of formula C5 were solubilized in 386,4 g (4.4 mol) of n-pentanol and added to the reactor. The reaction mixture was stirred at 1000 rpm at 25° C. while bubbling 40 l/h of air through the mixture for 8 h. The aqueous phase was separated, and the organic phase was washed three times with 170 ml water at 25° C. The solvent was removed at 100° C./10×10² Pa and the product further degassed at 100° C./2×10² Pa yielding 94,0% of quinone C33 as determined by HPLC-w %.

### CN77, EXAMPLE 1054, CF. CN24

### Moderate Temperature, Semi-Batchwise Synthesis of α-Tocopherol Quinone of Formula C33

### CN78, EXAMPLE 879, CF. CN60

### Moderate Temperature, Semi-Batchwise Synthesis of α-Tocopherol Quinone of Formula C33

### CN79, EXAMPLE 994

### Moderate Temperature, Batchwise Synthesis of α-Tocopherol Quinone of Formula C32

13,32 g (78.13 mmol) of CuCl₂×2 H₂O, CAS no: 10125-13-0 was dissolved in 28,15 g (1.56 mol) of water and placed in the reactor. 134,60 g (312.51 mmol) of α-tocopherol of formula C3 were solubilized in 386,32 g (3.78 mol) of n-hexanol and added to the reactor. The reaction mixture was stirred at 1000 rpm at 25° C. while bubbling 40 l/h of air through it for a period of 7 h. The aqueous phase was removed, and the organic phase was washed three times with water. The at least one solvent or the C-bearing solvent of the organic phase was removed under reduced pressure at 80° C. yielding 145,3 g corresponding to a yield of 92,1% as determined by. The product was degassed at 110° C. and 2,3×102 Pa and the amount of organic chlorine was determined to be 126 ppm, the amount of chloride 14 ppm and the amount of Cu was 49 ppm.

### CN80, EXAMPLE 1032, CF. CN25

### Moderate Temperature, Semi-Batchwise Synthesis of α-Tocopherol Quinone of Formula C33

### CN81, EXAMPLE 1042

### Influence of Reaction Temperature and Reaction Time on Formation of Reagent Traces and Side-Product Traces, Semi-Batchwise Synthesis of α-Tocopherol Quinone of Formula C33

13,32 g (78.13 mmol) of CuCl₂×2 H₂O, CAS no: 10125-13-0 were dissolved in 28,15 g (1.56 mol) of water and placed in the reactor, which was thereafter supplemented with 87,5 g (856.42 mmol) of n-hexanol. The reaction mixture was maintained at 25° C. under stirring at 1000 rpm while bubbling 40l/h of air through it. 134,60 g (312.51 mmol) of α-tocopherol of formula C5 were solubilized in 298,87 g (2.93 mol) of n-hexanol and added dropwise into the reactor during 4 h while stirring and bubbling. After a further two hours of reaction, the aqueous phase was removed. The organic phase was washed three times with 170 ml of water at 40-47° C. Removal of solvent from the organic Phase yielded 98,6% of α-tocopherol quinone of formula C33 as determined by HPLC-w %. By the methods indicated supra, the amount of organic chlorine was determined to be 88 ppm, the amount of chloride was determined to be 12 ppm and the amount of Cu ions was determined to be 8 ppm.

### CN82, EXAMPLE 1032, CF. CN25

### Influence of Reaction Temperature and Reaction Time on Formation of Reagent Traces and Side-Product Traces, Semi-Batchwise Synthesis of α-Tocopherol Quinone of Formula C33

This example per se is an inventive example, however, serves as comparison with respect to reaction temperature and reaction time on the aforementioned trace-formation.

### CN83, EXAMPLE 1036

### Influence of Reaction Temperature and Reaction Time on Formation of Reagent Traces and Side-Product Traces, Semi-Batchwise Synthesis of α-Tocopherol Quinone of Formula C33

This example per se is an inventive example, however, serves as comparison with respect to reaction temperature and reaction time on the aforementioned trace-formation.

13,32 g (78.13 mmol) of CuCl₂×2 H₂O, CAS no: 10125-13-0 were dissolved in 28,15 g (1.56 mol) of water and placed in the reactor, which was thereafter supplemented with 87,5 g (671.89 mmol) of 2-ethylhexanol. The reaction mixture was maintained at 55° C. under stirring at 1000 rpm while bubbling 40 l/h of air through it. 134,60 g (312.51 mmol) of α-tocopherol of formula C5 were solubilized in 298,72 g (2.29 mol) of 2-ethylhexanol and added dropwise into the reactor during 4 h while stirring and bubbling. After a further hour of reaction, the aqueous phase was removed. The organic phase was washed three times with 170 ml of water at 48° C. A sample of the combined organic phases was taken and revealed 98% of α-tocopherol quinone of formula C33 as determined by HPLC. The solvent was removed from the organic phase, and by the methods indicated supra, the amount of organic chlorine was determined to be 356 ppm, the amount of chloride was determined to be 34 ppm and the amount of Cu ions was determined to be 40 ppm.

### CN84, EXAMPLE 886

### Influence of Reaction Temperature and Reaction Time on Formation of Reagent Traces and Side-Product Traces, Batchwise Synthesis of α-Tocopherol Quinone of Formula C33

13,32 g (78.1 mmol) of CuCl₂×2 H₂O, CAS no: 10125-13-0 were dissolved in 28,2 g (1.6 mol) of water and placed in the reactor. 143,2 g (312.5 mmol) of α-tocopherol of formula C5 were solubilized in 386,4 g (3.8 mol) of n-hexanol and added to the reactor. The reaction mixture was stirred at 1000 rpm at 10° C. while bubbling 40 l/h of air through the mixture for 8 h. The mixture was washed three times with 170 ml water at 45-50° C. and the solvent removed at 100° C./10×10² Pa. The product was further degassed at 100° C./1×10² Pa yielding 95,6% of α-tocopherol quinone C33 as determined by HPLC-w %. By the methods indicated supra, the amount of organic chlorine was determined to be 70 ppm, the amount of chloride was determined to be 80 ppm and the amount of Cu ions was determined to be 95 ppm.

### CN85, EXAMPLE 877, CF. CN26

### Influence of Reaction Temperature and Reaction Time on Formation of Reagent Traces and Side-Product Traces, Batchwise Synthesis of α-Tocopherol Quinone of Formula C33

### CN86, EXAMPLE 883, CF. CN70

### Influence of Reaction Temperature and Reaction Time on Formation of Reagent Traces and Side-Product Traces, Batchwise Synthesis of α-Tocopherol Quinone of Formula C33

### CN87, EXAMPLE 1080

### Influence of Reaction Temperature and Reaction Time on Formation of Reagent Traces and Side-Product Traces, Batchwise Synthesis of α-Tocopherol Quinone of Formula C33

13,32 g (78.1 mmol) of CuCl₂×2 H₂O, CAS no: 10125-13-0 were dissolved in 28,2 g (1.6 mol) of water and placed in the reactor. 144,2 g (312.5 mmol) of α-tocopherol of formula C5 were solubilized in 386,4 g (3.8 mol) of n-hexanol and added to the reactor. The reaction mixture was stirred at 1000 rpm at 25° C. while bubbling 40 l/h of air through the mixture for 4,75 h. The aqueous phase was separated, and the organic phase was washed three times with 170 ml water at 47-49° C. The solvent was removed at 100° C./10×10² Pa and the product further degassed at 100° C./1×10² Pa yielding 94,5% of quinone C33 as determined by HPLC-w %. By the methods indicated supra, the amount of organic chlorine was determined to be 44 ppm, the amount of chloride was determined to be 32 ppm and the amount of Cu ions was determined to be 30 ppm.

### CN88, EXAMPLE 905, CF. CN14

### Influence of Reaction Temperature and Reaction Time on Formation of Reagent Traces and Side-Product Traces, Batchwise Synthesis of α-Tocopherol Quinone of Formula C33

### CN89, EXAMPLE 1054, CF. CN24

### Influence of Reaction Temperature and Reaction Time on Formation of Reagent Traces and Side-Product Traces, Batchwise Synthesis of α-Tocopherol Quinone of Formula C33

### CN90, EXAMPLE 879, CF. CN60

### Influence of Reaction Temperature and Reaction Time on Formation of Reagent Traces and Side-Product Traces, Batchwise Synthesis of α-Tocopherol Quinone of Formula C33

### CN91, EXAMPLE 1024, CF. CN68

### Influence of Reaction Temperature and Reaction Time on Formation of Reagent Traces and Side-Product Traces, Batchwise Synthesis of α-Tocopherol Quinone of Formula C33

This example per se is an inventive example, however, serves as comparison with respect to reaction temperature and reaction time on the aforementioned trace-formation.

### CN92, EXAMPLE 1040

### Influence of Reaction Temperature and Reaction Time on Formation of Reagent Traces and Side-Product Traces, Batchwise Synthesis of α-Tocopherol Quinone of Formula C33

This example per se is an inventive example, however, serves as comparison with respect to reaction temperature and reaction time on the aforementioned trace-formation.

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,42 g (3.78 mol) of n-hexanol and added to the reactor. The reaction mixture was stirred at 1000 rpm at 25° C. while bubbling 40 l/h of air through it for a period of 23 h. The aqueous phase was removed. The organic phase was washed three times with water and the at least one solvent or the C-bearing solvent of the organic phase removed at 90° C. during 45 min under reduced pressure. A sample was taken and revealed a yield of 96% of quinone C33 as determined by HPLC-w %. By the methods indicated supra, the amount of organic chlorine was determined to be 293 ppm, the amount of chloride was determined to be 27 ppm and the amount of Cu ions was determined to be 23 ppm.

### CN93, EXAMPLE 1010

### Influence of Reaction Temperature and Reaction Time on Formation of Reagent Traces and Side-Product Traces, Batchwise Synthesis of α-Tocopherol Quinone of Formula C33

This example per se is an inventive example, however, serves as comparison with respect to reaction temperature and reaction time on the aforementioned trace-formation.

13,32 g (78.1 mmol) of CuCl₂×2 H₂O, CAS no: 10125-13-0 were dissolved in 28,2 g (1.6 mol) of water and placed in the reactor. 144,2 g (312.5 mmol) of α-tocopherol of formula C5 were solubilized in 386,4 g (3.8 mol) of n-hexanol and added to the reactor. The reaction mixture was stirred at 1000 rpm at 40° C. while bubbling 40 l/h of air through the mixture for 5 h. The aqueous phase was separated, and the organic phase washed three times with 170 ml water at 40° C. The solvent was removed at 90° C./2×10² Pa yielding 148,9 g, 88,8 t % of α-tocopherol quinone of formula C33 as determined by HPLC-w %. By the methods indicated supra, the amount of organic chlorine was determined to be 250 ppm, the amount of chloride was determined to be 70 ppm and the amount of Cu ions was determined to be 100 ppm.

### CN94, EXAMPLE 1044 WITH SAMPLE FROM EXAMPLE 1042

### Influence of the Separation Means on Formation of Reagent Traces and Side-Product Traces, Semi-Batchwise Synthesis of α-Tocopherol Quinone of Formula C33

A G3 glass suction filter (volume of 1 l, 12.5 cm inner diameter) was filled with a slurry of 355 g of silica (particle size 40 to 63 μm) in n-heptane to a height of 6.7 cm giving a volume of 822 ml. 35,5 g of α-tocopherol quinone of formula C33 of example 1042 (cf. CN81) were dissolved in 35,5 g of n-heptane and applied onto the wet silica. Under suction another 1000 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 34,1 g of α-tocopherol quinone of formula C33 (quinone preparation of the invention). The amount of organic chlorine in said quinone preparation was 16 ppm, the amount of chloride <3 ppm and the amount of Cu was <3 ppm

### CN95, EXAMPLE 1033 WITH SAMPLE FROM EXAMPLE 1032

### Influence of the Separation Means on Formation of Reagent Traces and Side-Product Traces, Semi-Batchwise Synthesis of α-Tocopherol Quinone of Formula C33

A G3 glass suction filter (volume of 1 l, 12.5 cm inner diameter) was filled with a slurry of 355 g silica (particle size 40 to 63 μm) in n-heptane to a height of 6.7 cm giving a volume of 822 ml. 35,5 g of α-tocopherol quinone of formula C33 of example 1032 (cf. CN25) were dissolved in 35,5 g of n-heptane and applied onto the wet silica. Under suction another 1000 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 34,1 g of α-tocopherol quinone of formula C33 quinone preparation of the invention. The amount of organic chlorine in said quinone preparation was 76 ppm, the amount of chloride <3 ppm and the amount of Cu was <3 ppm.

### CN96, EXAMPLE 1038 WITH SAMPLE FROM EXAMPLE 1036

### Influence of the Separation Means on Formation of Reagent Traces and Side-Product Traces, Semi-Batchwise Synthesis of α-Tocopherol Quinone of Formula C33

A G3 glass suction filter (volume of 1 l, 12.5 cm inner diameter) was filled with a slurry of 355 g silica (particle size 40 to 63 μm) in n-heptane to a height of 6.7 cm giving a volume of 822 ml. 35,5 g of α-tocopherol quinone of formula C33 of example 1036 (cf. CN83) were dissolved in 35,5 g of n-heptane and applied onto the wet silica. Under suction another 1000 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 30,0 g of α-tocopherol quinone of formula C33 (quinone preparation of the invention). The amount of organic chlorine in said quinone preparation was 210 ppm, the amount of chloride <3 ppm and the amount of Cu was <3 ppm.

### CN97, EXAMPLE 895 WITH SAMPLE FROM EXAMPLE 886

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 300 g silica (particle size 40 to 63 μm) in n-heptane to a height of 6.5 cm. 30,0 g of α-tocopherol quinone of formula C33 of example 886 (cf. CN84) were dissolved in 13 g of n-heptane and applied onto the wet silica. Under suction another 500 ml of n-heptane were added. Thereafter elution was realized two times with 2.403 g of a solution of n-heptane comprising 3 w % of isopropyl acetate yielding fractions 1 and 2 followed by one elution with 2.403 g of a solution of n-heptane containing 20 w % of isopropyl acetate yielding fraction 3. Said fraction 3 was freed from solvent and dried to give 25,9 g of α-tocopherol quinone of formula C33 (quinone preparation of the invention). The amount of organic chlorine in said quinone preparation was 12 ppm, the amount of chloride <1 ppm and the amount of Cu was <3 ppm.

### CN98, EXAMPLE 1027 WITH SAMPLE FROM EXAMPLE 1024

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 355 g silica (particle size 40 to 63 μm) in n-heptane to a height of 6.7 cm giving a volume of 822 ml. 35,5 g of α-tocopherol quinone of formula C33 of example 1024 (cf. CN68) were dissolved in 14,2 g of n-heptane and applied onto the wet silica. Under suction another 1500 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 33,8 g of α-tocopherol quinone of formula C33 (quinone preparation of the invention). The amount of organic chlorine in said quinone preparation was 20 ppm, the amount of chloride <3 ppm and the amount of Cu was <3 ppm

### CN99, EXAMPLE 1092 WITH SAMPLE FROM EXAMPLE 1091

### Influence of the Separation Means on Formation of Reagent Traces and Side-Product Traces, Batchwise Synthesis of α-Tocopherol Quinone of Formula C33

### EXAMPLE 1091

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,42 g (3.78 mol) of n-hexanol and added to the reactor. The reaction mixture was stirred at 1000 rpm at 15° C. while bubbling 40 l/h of air through it for a period of 4,75 h. The aqueous phase was removed. The organic phase was washed three times with 170 ml of water at 40° C. The solvent of the organic phase was removed at 100° C./10×10² Pa and the product was further degassed at 100° C./1×10² Pa yielding 87,4% of quinone C33 as determined by HPLC-w %. By the methods indicated supra, the amount of organic chlorine was determined to be 10 ppm, the amount of chloride was determined to be 140 ppm and the amount of Cu ions was determined to be 160 ppm.

### EXAMPLE 1092

A G3 glass suction filter (volume of 1 l, 12.5 cm inner diameter) was filled with a slurry of 355 g of silica (particle size 40 to 63 μm) in n-heptane to a height of 6.7 cm giving a volume of 822 ml. 35,5 g of α-tocopherol quinone of formula C33 of example 1091 were dissolved in 14,2 g of n-heptane and applied onto the wet silica. Under suction another 500 ml of n-heptane were added. Thereafter elution was realized two times with 2,428 g of a solution of n-heptane comprising 3 w % of isopropyl acetate yielding fractions 1 and 2 followed by one elution with 2,428 g of a solution of n-heptane containing 20 w % of isopropyl acetate yielding fraction 3. Said fraction 3 was freed from solvent and dried to give 29,3 g of α-tocopherol quinone of formula C33 (quinone preparation of the invention). The amount of organic chlorine in said quinone preparation was 5 ppm, the amount of chloride <3 ppm and the amount of Cu was 7 ppm

### CN100, EXAMPLE 880 WITH SAMPLE FROM EXAMPLE 877

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 300 g of silica (particle size 40 to 63 μm) in n-heptane to a height of 6.5 cm. 29,9 g of α-tocopherol quinone of formula C33 of example 877 (cf. CN26) were dissolved in 14 g of n-heptane and applied onto the wet silica. Under suction another 500 ml of n-heptane were added. Thereafter elution was realized two times with 2.056 g of a solution of n-heptane comprising 3 w % of isopropyl acetate yielding fractions 1 and 2 followed by one elution with 2.052 g of a solution of n-heptane containing 20 w % of isopropyl acetate yielding fraction 3. Said fraction 3 was freed from solvent and dried to give 26,3 g of α-tocopherol quinone of formula C33 (quinone preparation of the invention). The amount of organic chlorine in said quinone preparation was 14 ppm, the amount of chloride <1 ppm and the amount of Cu was <3 ppm.

### CN101, EXAMPLE 1019 WITH SAMPLE FROM EXAMPLE 1014

### Influence of the Separation Means on Formation of Reagent Traces and Side-Product Traces, Batchwise Synthesis of α-Tocopherol Quinone of Formula C33

### EXAMPLE 1014

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,42 g (3.78 mol) of n-hexanol and added to the reactor. The reaction mixture was stirred at 1000 rpm at 25° C. while bubbling 40 l/h of air through it for a period of 4,75 h. The aqueous phase was removed. The organic phase was washed two times with 170 ml of water at 40° C. The organic phase was washed once more with 170 ml of water at 50° C. and 200 ml n-heptane were added for phase separation. The aqueous phase was washed with 400 ml n-heptane at 50° C. The solvent was removed from the combined organic phases at 90° C. under reduced pressure yielding 141,5 g (95,8%) quinone of formula C33 as determined by HPLC-w %. By the methods indicated supra, the amount of organic chlorine was determined to be 77 ppm, the amount of chloride was determined to be 77 ppm and the amount of Cu ions was determined to be <3 ppm.

### EXAMPLE 1019

A G3 glass suction filter (volume of 1 l, 12.5 cm inner diameter) was filled with a slurry of 355 g silica (particle size 40 to 63 μm) in n-heptane to a height of 6.7 cm giving a volume of 822 ml. 35,5 g of α-tocopherol quinone of formula C33 of example 1014 (cf. CN101) were dissolved in 14,2 g of n-heptane and applied onto the wet silica. Under suction another 1500 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 34,4 g of α-tocopherol quinone of formula C33 (quinone preparation of the invention). The amount of organic chlorine in said quinone preparation was 15 ppm, the amount of chloride <3 ppm and the amount of Cu was <3 ppm.

### CN102, EXAMPLE 1052, 1053, 1056, CF. CN1, CN2, CN3

### Influence of the Separation Means on Formation of Reagent Traces and Side-Product Traces, Batchwise Synthesis of α-Tocopherol Quinone of Formula C33

### CN103, EXAMPLE 908 WITH SAMPLE FROM EXAMPLE 905

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 305 g silica (particle size 40 to 63 μm) in n-heptane to a height of 6.5 cm. 30,3 g of α-tocopherol quinone of formula C33 of example 905 (cf. CN14) were dissolved in 13 g of n-heptane and applied onto the wet silica. Under suction another 500 ml of n-heptane were added. Thereafter elution was realized two times with 2.443 g of a solution of n-heptane comprising 3 w % of isopropyl acetate yielding fractions 1 and 2 followed by one elution with 2.443 g of a solution of n-heptane containing 20 w % of isopropyl acetate yielding fraction 3. Said fraction 3 was freed from solvent and dried to give 26,6 g of α-tocopherol quinone of formula C33 (quinone preparation of the invention). The amount of organic chlorine in said quinone preparation was 32 ppm, the amount of chloride <1 ppm and the amount of Cu was <3 ppm.

### CN104, EXAMPLE 909 WITH SAMPLE FROM EXAMPLE 906

### Influence of the Separation Means on Formation of Reagent Traces and Side-Product Traces, Batchwise Synthesis of α-Tocopherol Quinone of Formula C33

### EXAMPLE 906

13,32 g (78.1 mmol) of CuCl₂×2 H₂O, CAS no: 10125-13-0 were dissolved in 28,2 g (1.6 mol) of water and placed in the reactor. 143,2 g (312.5 mmol) of α-tocopherol of formula C5 were solubilized in 386,4 g (3.8 mol) of n-hexanol and added to the reactor. The reaction mixture was stirred at 1000 rpm at 25° C. while bubbling 40 l/h of air through the mixture for 6 h. The aqueous phase was separated, and the organic phase was washed three times with water at 25° C. The solvent was removed at 100° C./8×10² Pa and the product further degassed at 100° C./2×10² Pa yielding 100% of quinone C33 as determined by HPLC-w %. By the methods indicated supra, the amount of organic chlorine was determined to be 69 ppm, the amount of chloride was determined to be 27 ppm and the amount of Cu ions was determined to be 13 ppm.

### EXAMPLE 909

A G3 glass suction filter (volume of 1 l, 12.5 cm inner diameter) was filled with a slurry of 305 g silica (particle size 40 to 63 μm) in n-heptane to a height of 6.5 cm. 30,7 g of the quinone prepared above were dissolved in 13 g of n-heptane and applied onto the wet silica. Under suction another 500 ml of n-heptane were added. Thereafter elution was realized two times with 2.443 g of a solution of n-heptane comprising 3 w % of isopropyl acetate yielding fractions 1 and 2 followed by one elution with 2.443 g of a solution of n-heptane containing 20 w % of isopropyl acetate yielding fraction 3. Said fraction 3 was freed from solvent and dried to give 27,2 g of α-tocopherol quinone of formula C33 (quinone preparation of the invention). The amount of organic chlorine in said quinone preparation was 34 ppm, the amount of chloride <1 ppm and the amount of Cu was <3 ppm.

### CN105, EXAMPLE 1049 WITH SAMPLE FROM EXAMPLE 1040

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 355 g silica (particle size 40 to 63 μm) in n-heptane to a height of 6.7 cm giving a volume of 822 ml. 35,5 g of α-tocopherol quinone of formula C33 of example 1040 (cf. CN92) were dissolved in 14,2 g of n-heptane and applied onto the wet silica. Under suction another 1000 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 33,8 g of α-tocopherol quinone of formula C33 (quinone preparation of the invention). The amount of organic chlorine in said quinone preparation was 170 ppm, the amount of chloride <3 ppm and the amount of Cu was <3 ppm.

### CN106, EXAMPLE 1012 WITH SAMPLE FROM EXAMPLE 1010

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 355 g of silica (particle size 40 to 63 μm) in n-heptane to a height of 6.7 cm giving a volume of 822 ml. 35,5 g of α-tocopherol quinone of formula C33 of example 1010 (cf. CN93) were dissolved in 14,2 g of n-heptane and applied onto the wet silica. Under suction another 2000 ml of n-heptane were added. Thereafter elution was realized two times with 3550 ml of a solution of n-heptane comprising 3 w % of isopropyl acetate yielding fractions 1 and 2 followed by one elution with 3550 ml of a solution of n-heptane containing 20 w % of isopropyl acetate yielding fraction 3. Said fraction 3 was freed from solvent and dried to give 33,9 g of α-tocopherol quinone of formula C33 (quinone preparation of the invention). The amount of organic chlorine in said quinone preparation was 65 ppm, the amount of chloride <3 ppm and the amount of Cu was <3 ppm.

### CN107, EXAMPLE 1057 WITH SAMPLE FROM EXAMPLE 1054

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of silica (particle size 40 to 63 μm) in n-heptane to a height of 6.7 cm giving a volume of 802 ml. 35,5 g of α-tocopherol quinone of formula C33 of example 1054 (cf. CN24) were dissolved in 14,2 g of n-heptane and applied onto the wet silica. Under suction another 1000 ml of n-heptane were added. Thereafter elution was realized two times with 2.428 g of a solution of n-heptane comprising 3 w % of isopropyl acetate yielding fractions 1 and 2 followed by one elution with 2.428 g of a solution of n-heptane containing 20 w % of isopropyl acetate yielding fraction 3. Said fraction 3 was freed from solvent and dried to give 34,1 g of α-tocopherol quinone of formula C33 (quinone preparation of the invention). The amount of organic chlorine in said quinone preparation was 9 ppm, the amount of chloride <3 ppm and the amount of Cu was <3 ppmcn

### CN108, EXAMPLE 885 WITH SAMPLE FROM EXAMPLE 8791

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 300 g of silica (particle size 40 to 63 μm) in n-heptane to a height of 6.5 cm giving a volume of 802 ml. 29,9 g of α-tocopherol quinone of formula C33 of example 879 (cf. CN60) were dissolved in 13 g of n-heptane and applied onto the wet silica. Under suction another 500 ml of n-heptane were added. Thereafter elution was realized two times with 2.403 g of a solution of n-heptane comprising 3 w % of isopropyl acetate yielding fractions 1 and 2 followed by one elution with 2.403 g of a solution of n-heptane containing 20 w % of isopropyl acetate yielding fraction 3. Said fraction 3 was freed from solvent and dried to give 26,9 g of α-tocopherol quinone of formula C33 (quinone preparation of the invention). The amount of organic chlorine in said quinone preparation was 36 ppm, the amount of chloride <1 ppm and the amount of Cu was <3 ppm.

### CN109, EXAMPLE 1087 WITH SAMPLE FROM EXAMPLE 1086

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 355 g of silica (particle size 40 to 63 μm) in n-heptane to a height of 6.7 cm giving a volume of 822 ml. 35,5 g of α-tocopherol quinone of formula C33 of example 1086 (cf. CN16) were dissolved in 14,2 g of n-heptane and applied onto the wet silica. Under suction another 500 ml of n-heptane were added. Thereafter elution was realized two times with 2.428 g of a solution of n-heptane comprising 3 w % of isopropylacetate yielding fractions 1 and 2 followed by one elution with 2.428 g of a solution of n-heptane containing 20 w % of isopropyl acetate yielding fraction 3. Said fraction 3 was freed from solvent and dried to give 33,3 g of α-tocopherol quinone of formula C33 (quinone preparation of the invention). The amount of organic chlorine in said quinone preparation was 7 ppm, the amount of chloride <3 ppm and the amount of Cu was <3 ppm

### CN110, EXAMPLE 1008 WITH SAMPLE FROM EXAMPLE 994

### Influence of the Separation Means on Formation of Reagent Traces and Side-Product Traces, Batchwise Synthesis of α-Tocopherol Quinone of Formula C33

A G3 glass suction filter (volume of 1 l, 12.5 cm inner diameter) was filled with a slurry of 355 g of silica (particle size 40 to 63 μm) in n-heptane to a height of 6.7 cm giving a volume of 822 ml. 35,5 g of α-tocopherol quinone of formula C32 of example 994 (cf. CN79) were dissolved in 14,2 g of n-heptane and applied onto the wet silica. Under suction another 2500 ml of n-heptane were added. Thereafter elution was realized two times with 3550 ml of a solution of n-heptane comprising 3 w % of isopropyl acetate yielding fractions 1 and 2 followed by one elution with 3550 ml of a solution of n-heptane containing 20 w % of isopropyl acetate yielding fraction 3. Said fraction 3 was freed from solvent and dried to give 32,1 g of α-tocopherol quinone of formula C32 (quinone preparation of the invention). The amount of organic chlorine in said quinone preparation was 47 ppm, the amount of chloride <3 ppm and the amount of Cu was <3 ppm.

### CN111, EXAMPLE 1043 WITH SAMPLE FROM EXAMPLE 1042

### Influence of Another Distillation Step on Formation of Reagent Traces and Side-Product Traces, Semi-Batchwise Synthesis of α-Tocopherol Quinone of Formula C33

54,9 g of α-tocopherol quinone of formula C33 from example 1042 (cf. CN81) were distilled at 110° C. and at a vacuum of 2,3*10² Pascal. 32,17 g of the bottom fraction were diluted with 3,57 g of sunflower oil and distilled at 190° C. and 4 Pascal yielding 24,9 g. By the methods indicated supra, the amount of trace components in said quinone preparation was determined as follows: Organic chlorine in said quinone preparation was 51 ppm, the amount of chloride <3 ppm and the amount of Cu was 2 ppm.

### CN112, EXAMPLE 1034 WITH SAMPLE FROM EXAMPLE 1032

### Influence of Another Distillation Step on Formation of Reagent Traces and Side-Product Traces, Semi-Batchwise Synthesis of α-Tocopherol Quinone of Formula C33

41,4 g of α-tocopherol quinone of formula C33 from example 1032 (cf. CN25) were distilled at 110° C. and at a vacuum of 2,3×10² Pa. 24,5 g of the bottom fraction were diluted with 2,7 g of sunflower oil. 24,3 g of this mixture were distilled at 190° C. and 3 Pa yielding 18,7 g. By the methods indicated supra, the amount of trace components in said quinone preparation was determined as follows: Organic chlorine in said quinone preparation was 115 ppm, the amount of chloride 5 ppm and the amount of Cu was 14 ppm.

### CN113, EXAMPLE 1039 WITH SAMPLE FROM EXAMPLE 1036

### Influence of Another Distillation Step on Formation of Reagent Traces and Side-Product Traces, Semi-Batchwise Synthesis of α-Tocopherol Quinone of Formula C33

97,2 g of α-tocopherol quinone of formula C33 from example 1036 (cf. CN83) were distilled at 130° C. and at a vacuum of 2,3×10² Pa. 24,8 g of the bottom fraction were diluted with 2,8 g of sunflower oil. 24,5 g of this mixture were distilled at 190° C. and 3 Pa yielding 17,8 g. By the methods indicated supra, the amount of trace components in said quinone preparation was determined as follows: Organic chlorine in said quinone preparation was 321 ppm, the amount of chloride 9 ppm and the amount of Cu was 24 ppm.

### CN114, EXAMPLE 887 WITH SAMPLE FROM EXAMPLE 886

### Influence of Another Distillation Step on Formation of Reagent Traces and Side-Product Traces, Batchwise Synthesis of α-Tocopherol Quinone of Formula C33

23,4 g of α-tocopherol quinone of formula C33 from example 886 (cf. CN84) were diluted with 2,5 g of sunflower oil. 24,6 g of this mixture were distilled at 190° C. and 2.3 Pa yielding 19,4 g. By the methods indicated supra, the amount of trace components in said quinone preparation was determined as follows: Organic chlorine in said quinone preparation was 77 ppm, the amount of chloride 8 ppm and the amount of Cu was 41 ppm.

### CN115, EXAMPLE 1028 WITH SAMPLE FROM EXAMPLE 1024

### Influence of Another Distillation Step on Formation of Reagent Traces and Side-Product Traces, Batchwise Synthesis of α-Tocopherol Quinone of Formula C33

52,7 g of α-tocopherol quinone of formula C33 from example 1024 (cf. CN68) were distilled at 110° C. and at a vacuum of 2,3×10² Pa. 34,9 g of the bottom fraction were diluted with 3,9 g of sunflower oil. 27,6 g of this mixture were distilled at 190° C. and 6 Pa yielding 21,3 g. By the methods indicated supra, the amount of trace components in said quinone preparation was determined as follows: Organic chlorine in said quinone preparation was 86 ppm, the amount of chloride 24 ppm and the amount of Cu was 39 ppm.

### CN116, EXAMPLE 878 WITH SAMPLE FROM EXAMPLE 877

### Influence of Another Distillation Step on Formation of Reagent Traces and Side-Product Traces, Batchwise Synthesis of α-Tocopherol Quinone of Formula C33

25,7 g of α-tocopherol quinone of formula C33 from example 877 (cf. CN26) were diluted with 2,9 g of sunflower oil. 27,6 g of this mixture were distilled at 190° C. and 2 Pa yielding 22,3 g. By the methods indicated supra, the amount of trace components in said quinone preparation was determined as follows: Organic chlorine in said quinone preparation was 18 ppm, the amount of chloride <1 ppm and the amount of Cu was <3 ppm.

### CN117, EXAMPLE 1016 WITH SAMPLE FROM EXAMPLE 1014

### Influence of Another Distillation Step on Formation of Reagent Traces and Side-Product Traces, Batchwise Synthesis of α-Tocopherol Quinone of Formula C33

93,8 g of α-tocopherol quinone of formula C33 from example 1014 (cf. CN101) were distilled at 110° C. and at a vacuum of 2,3*10² Pascal. 40,3 g of the bottom fraction were diluted with 4,5 g of sunflower oil and 42,9 g of this mixture distilled at 190° C. and 4 Pascal yielding 32,8 g of quinone C33. By the methods indicated supra, the amount of trace components in said quinone preparation was determined as follows: Organic chlorine in said quinone preparation was 38 ppm, the amount of chloride <3 ppm and the amount of Cu was 3 ppm.

### CN118, EXAMPLE 910 WITH SAMPLE FROM EXAMPLE 905

### Influence of Another Distillation Step on Formation of Reagent Traces and Side-Product Traces, Batchwise Synthesis of α-Tocopherol Quinone of Formula C33

26,6 g of α-tocopherol quinone of formula C33 from example 905 (cf. CN14) were distilled at 110° C. and at a vacuum of 2,3*10² Pascal. 32,46 g of the bottom fraction were diluted with 3,36 g of sunflower oil. 30,2 g of this mixture were distilled at 190° C. and 3,2 Pascal yielding 24,6 g. By the methods indicated supra, the amount of trace components in said quinone preparation was determined as follows: Organic chlorine in said quinone preparation was 74 ppm, the amount of chloride 7 ppm and the amount of Cu was 22 ppm.

### CN119, EXAMPLE 911 WITH SAMPLE FROM EXAMPLE 906

### Influence of Another Distillation Step on Formation of Reagent Traces and Side-Product Traces, Batchwise Synthesis of α-Tocopherol Quinone of Formula C33

24,6 g of α-tocopherol quinone of formula C33 from example 906 (cf. CN104) were diluted with 2,7 g of sunflower oil. 26,6 g of this mixture were distilled at 190° C. and 3 Pa yielding 21,4 g. By the methods indicated supra, the amount of trace components in said quinone preparation was determined as follows: Organic chlorine in said quinone preparation was 77 ppm, the amount of chloride 3 ppm and the amount of Cu was 11 ppm.

### CN120, EXAMPLE 1048 WITH SAMPLE FROM EXAMPLE 1040

### Influence of Another Distillation Step on Formation of Reagent Traces and Side-Product Traces, Batchwise Synthesis of α-Tocopherol Quinone of Formula C33

50 g of α-tocopherol quinone of formula C33 from example 1040 (cf. CN92) were distilled at 110° C. and at a vacuum of 2,3×10² Pa. 33,4 g of the bottom fraction were diluted with 3,7 g of sunflower oil. 32,9 g of this mixture were distilled at 190° C. and 5 Pa yielding 25,8 g. By the methods indicated supra, the amount of trace components in said quinone preparation was determined as follows: Organic chlorine in said quinone preparation was 239 ppm, the amount of chloride 11 ppm and the amount of Cu was 13 ppm.

### CN121, EXAMPLE 1011 WITH SAMPLE FROM EXAMPLE 1010

### Influence of Another Distillation Step on Formation of Reagent Traces and Side-Product Traces, Batchwise Synthesis of α-Tocopherol Quinone of Formula C33

30,4 g of α-tocopherol quinone of formula C33 from example 1010 (cf. CN93) were diluted with 3,4 g of sunflower oil. 31,9 g of this mixture were distilled at 190° C. and 4.5 Pa yielding 26,2 g. By the methods indicated supra, the amount of trace components in said quinone preparation was determined as follows: Organic chlorine in said quinone preparation was 131 ppm, the amount of chloride 29 ppm and the amount of Cu was 43 ppm.

### CN122, EXAMPLE 1055 WITH SAMPLE FROM EXAMPLE 1054

### Influence of Another Distillation Step on Formation of Reagent Traces and Side-Product Traces, Batchwise Synthesis of α-Tocopherol Quinone of Formula C33

107,2 g of α-tocopherol quinone of formula C33 from example 1054 (cf. CN24) were distilled at 110° C. and at a vacuum of 2,3×10² Pa. 24,7 g of the bottom fraction were diluted with 2,8 g of sunflower oil. 25,0 g of this mixture were distilled at 190° C. and 4 Pa yielding 16,9 g. By the methods indicated supra, the amount of trace components in said quinone preparation was determined as follows: Organic chlorine in said quinone preparation was 56 ppm, the amount of chloride 3 ppm and the amount of Cu was 6 ppm.

### CN123, EXAMPLE 881 FROM EXAMPLE 879

### Influence of Another Distillation Step on Formation of Reagent Traces and Side-Product Traces, Batchwise Synthesis of α-Tocopherol Quinone of Formula C33

26,8 g of α-tocopherol quinone of formula C33 from example 879 (cf. CN60) were diluted with 2,9 g of sunflower oil. 30,3 g of this mixture were distilled at 190° C. and 2.4 Pa yielding 25,1 g. By the methods indicated supra, the amount of trace components in said quinone preparation was determined as follows: Organic chlorine in said quinone preparation was 53 ppm, the amount of chloride <1 ppm and the amount of Cu was <3 ppm.

### CN124, EXAMPLE 1090 WITH SAMPLE FROM EXAMPLE 1089

### Influence of Another Distillation Step on Formation of Reagent Traces and Side-Product Traces, Batchwise Synthesis of α-Tocopherol Quinone of Formula C33

### EXAMPLE 1089

13,32 g (78.13 mmol) of CuCl₂×2 H₂O, CAS no: 10125-13-0 were dissolved in 28,15 g (1.56 mol) of water and placed in the reactor. 134,60 g (312.51 mmol) of α-tocopherol of formula C5 were solubilized in 386,36 g (3.78 mol) of n-hexanol and added into the reactor. The reaction mixture was stirred at 1000 rpm at 15° C. while bubbling 40 l/h of air through it for a period of 7 h. The aqueous phase was removed and the organic phase was washed three times with 170 ml of water at 42° C. to 51° C. The at least one solvent was removed from the organic phase at 100° C./10×10² Pa followed by another distillation at 100° C./1×10² Pa. yielding 93,7% of α-tocopherol quinone C33 as determined by HPLC-w %. By the methods indicated supra, the amount of organic chlorine was determined to be 26 ppm, the amount of chloride was determined to be 22 ppm and the amount of Cu ions was determined to be 9 ppm.

### EXAMPLE 1090

24,9 g of the α-tocopherol quinone C33 obtained in example 1089 were mixed with 2,76 g sunflower oil and 25,0 g of this mixture were distilled at 180° C./2 Pa yielding 20,12 g. By the methods indicated supra, the amount of trace components in said quinone preparation was determined as follows: Organic chlorine in said quinone preparation was 23 ppm, the amount of chloride <3 ppm and the amount of Cu was 3 ppm.

### CN125, EXAMPLE 992 FROM SAMPLE OF EXAMPLE 990

### Influence of Another Distillation Step on Formation of Reagent Traces and Side-Product Traces, Semi-Batchwise Synthesis of α-Tocopherol Quinone of Formula C32

### EXAMPLE 990

13,32 g (78.13 mmol) of CuCl₂×2 H₂O, CAS no: 10125-13-0 were dissolved in 28,15 g (1.56 mol) of water and placed in the reactor, which was thereafter supplemented with 87,5 g (856.42 mmol) of n-hexanol. The reaction mixture was maintained at 25° C. under stirring at 1000 rpm while bubbling 40l/h of air through it. 134,60 g (312.51 mmol) of α-tocopherol of formula C3 were solubilized in 298,87 g (2.93 mol) of n-hexanol and added dropwise into the reactor during 2 h while stirring and bubbling. After a further 3.3 hours of reaction with stirring and bubbling, the aqueous phase was removed. The organic phase was washed once with 170 ml of water while the pH was adjusted to pH=1 using 5,0 g of 10% aqueous hydrochloric acid. The phases were separated, and the organic phase was washed two times with 170 ml water with adjusting the pH to 7 at the second of these two washings. The solvent was removed at 80° C. under reduced pressure and the product further degassed at 110° C. and 2×10² Pa yielding 90,1% of α-tocopherol quinone of formula C32 as determined by HPLC-w %. By the methods indicated supra, the amount of organic chlorine was determined to be 115 ppm, the amount of chloride was determined to be 15 ppm and the amount of Cu ions was determined to be 23 ppm

### EXAMPLE 992

63,0 g of the α-tocopherol quinone C32 obtained in example 990 were mixed with 7,0 g of pluriol. 65,5 g of this mixture were distilled at 190° C. and 3 Pa yielding 40,9 g. By the methods indicated supra, the amount of trace components in said quinone preparation was determined as follows: Organic chlorine in said quinone preparation was 79 ppm, the amount of chloride was <3 ppm and the amount of Cu was <3 ppm.

### CN126, EXAMPLE 1046 WITH SAMPLE FROM EXAMPLE 1046A

### Influence of a Separation Column on Formation of Reagent Traces and Side-Product Traces, Semi-Batchwise Synthesis of α-Tocopherol Quinone of Formula C33

### EXAMPLE 1046A

13,32 g (78.13 mmol) of CuCl₂×2 H₂O, CAS no: 10125-13-0 were dissolved in 28,15 g (1.56 mol) of water and placed in the reactor, which was thereafter supplemented with 87,5 g (856.42 mmol) of n-hexanol. The reaction mixture was maintained at 25° C. under stirring at 1000 rpm while bubbling 40l/h of air through it. 134,60 g (312.51 mmol) of α-tocopherol of formula C5 were solubilized in 298,87 g (2.93 mol) of n-hexanol and added dropwise into the reactor during 4 h while stirring and bubbling. After a further two hours of reaction under stirring and bubbling, the aqueous phase was removed. The organic phase was washed three times with water. A sample of the combined organic phases was taken and revealed 99% of α-tocopherol quinone of formula C33 as determined by HPLC-w %. The solvent was removed from the combined organic phases.

### EXAMPLE 1046

A slurry of silica (particle size 40 to 63 μm) either in toluene or in a mixture of 80 w % of hexane 20 w % of isopropyl acetate was filled into a glass column with frit (0,1 I, d=1.7 cm). 140 g of α-tocopherol quinone of formula C33 as previously prepared were solubilized in either 140 g of toluene or 112 g of the mixture of 80 w % of hexane 20 w % isopropyl acetate and applied onto the column. Elution was realized with the same solvent. The solvent was removed from the fraction obtained. The amount of organic chlorine in said quinone preparation was 73 ppm, the amount of chloride 3 ppm and the amount of Cu was <3 ppm.

One observes the invention to be a process for the oxidation of at least one chroman C1 in a solvent mixture comprising at least two solvents or in a C-bearing solvent, with a gaseous compound comprising, essentially consisting of, or consisting of oxygen in the presence of a copper catalyst, said copper catalyst exhibiting the oxidation state (+1) or (+2). A further part of the invention is a composition comprising at least one chroman C1 and/or at least one quinone C30, a solvent mixture comprising at least two solvents or a C-bearing solvent, a copper catalyst, said copper catalyst exhibiting the oxidation state (+1) or (+2) and a gaseous compound comprising, essentially consisting or consisting of oxygen. A quinone preparation, a process of making same and its use are likewise a substantial part of the invention.

1.-22. (canceled)

23. Process for the oxidation of at least one chroman (C1) 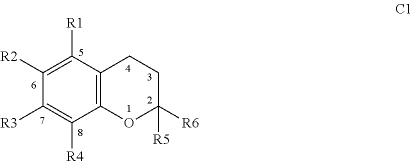
with R1, R3, R4, R5 being H or CH₃, R2 being OH, OAc, OCO—C₁-C₁₈-alkyl, and R6 being alkyl, alkenyl,
in a solvent mixture comprising at least two solvents or in a C-bearing solvent, with a gaseous compound comprising oxygen in the presence of a copper catalyst, said copper catalyst exhibiting the oxidation state (+1) or (+2).

24. The process according to claim 23, wherein the gaseous compound comprising oxygen is actively moved through the solvent mixture comprising at least two solvents or through the C-bearing solvent.

25. The process according to claim 23, wherein the copper catalyst is used in an amount ranging from 0,001 to 10 molar equivalents with respect to the molar amount of chroman (C1) used.

26. The process according to claim 23, wherein the copper catalyst is a copper halide.

27. The process according to claim 23, wherein the copper catalyst is combined with at least one metal compound selected form the group consisting of Na, Li, K, Cs, Mg, Ca, Sr, Ba, Fe, Cr, Mn, Co, Ni, Zn, La, Ce, Pr, Nd compounds.

28. The process according to claim 23, wherein the chroman (C1) is at least one of the group consisting of α-tocopherol of formula (C3), (C4), (C5) and α-tocotrienol of formula (C12), (C13), (C14).

29. The process according to claim 23, wherein the solvent mixture comprising at least two solvents or the C-bearing solvent is free of any detergent.

30. The process according to claim 23, wherein the at least two solvents of the solvent mixture comprise water and an organic solvent.

31. The process according to claim 30, wherein the at least two solvents of the solvent mixture comprise as organic solvent
at least one primary alcohol or
at least one secondary alcohol or
a mixture of at least one primary and at least one secondary alcohol

32. The process according to claim 30, wherein the weight ratio of the organic solvent to water ranges from 0.01:1 to 499:1.

33. Composition comprising:
a) at least one chroman (C1) 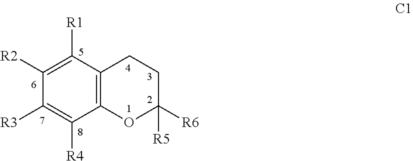
with R1, R3, R4, R5 being H or CH₃, R2 being OH, OAc, OCO—C₁-C₁₈-alkyl, and R6 being alkyl, alkenyl
and/or at least one quinone (C30) 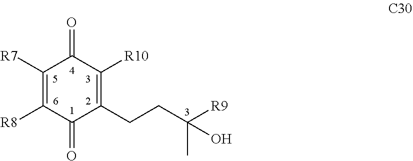
with R7, R8, R10 being H or CH₃; R9 being alkyl, alkenyl;
b) a solvent mixture comprising at least two solvents or a C-bearing solvent;
c) a copper catalyst, said copper catalyst exhibiting the oxidation state (+1) or (+2);
d) a gaseous compound comprising, essentially consisting or consisting of oxygen;
said composition being obtained by the process according to claim 23.

34. Composition according to claim 33, wherein the gaseous compound in the composition is in the form of gas bubbles, the amount of which is
higher than that amount, which is obtained, when a) to c) are combined and stored under ambient air.

35. Process for obtaining a quinone preparation comprising the steps:
i) removing one solvent from the solvent mixture comprising at least two solvents of the composition of claim 33, or removing the C-bearing solvent of the composition of claim 33;
with optionally adding hydrochloric acid prior or during
removing one solvent from the solvent mixture or
removing the C-bearing solvent;
iia) distilling off remaining solvent(s) or
iib) degassing the composition or
iic) distilling off remaining solvent(s) and degassing the composition;
iii) applying the composition of step iia), step iib) or step iic) onto a separation means, the diameter of the surface of said separation means being larger than the height of said separation means;
iv) optionally subjecting the remainder from step iii) to a further distillation,
or
i) removing one solvent from the solvent mixture comprising at least two solvents of the composition of claim 33, or removing the C-bearing solvent of the composition of claim 33.
with optionally adding hydrochloric acid prior or during
removing one solvent from the solvent mixture or
removing the C-bearing solvent;
iia) distilling off remaining solvent(s) or
iib) degassing the composition or
iic) distilling off remaining solvent(s) and degassing the composition;
iii) applying the composition of step iia), step iib) or step iic) to another distillation step;
iv) optionally subjecting the remainder from step iii) to a further distillation,
or
i) removing one solvent from the solvent mixture comprising at least two solvents of the composition of claim 33, or removing the C-bearing solvent of the composition of claim 33
with optionally adding hydrochloric acid prior or during
removing one solvent from the solvent mixture or
removing the C-bearing solvent;
iia) distilling off the remaining solvent(s); or
iib) degassing the composition; or
iic) distilling off remaining solvent(s) and degassing the composition;
iii) applying the composition of step iia), step iib) or step iic) onto a separation column;
iv) optionally subjecting the remainder from step iii) to a further distillation.

36. The process according to claim 35, wherein after step i) it comprises:
ia) reducing the volume of the removed one solvent from the composition and/or;
ib) adding hydrochloric acid to said removed one solvent;
ic) storing or reinjecting the thus obtained mixture of step ia) or ib),
or
id) adding hydrochloric acid to the removed one solvent from the composition and/or;
ie) reducing the volume of the mixture obtained in step id);
if) storing or reinjecting the thus obtained mixture of step id) or ie).

37. The process according to claim 35, wherein the separation means or the separation column comprises a solid support, said solid support being selected from at least one of silica, silica based material also named modified silica, zeolite, aluminum oxide, alumina silicates, carbon, carbon based materials, carbohydrate, polymeric organic materials, acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid, malic acid, or nitrilotriacetic acid (NTA), preferably being silica.

38. The process according to claim 37, wherein the solid support, preferably silica, has
a particle size ranging from 5 μm to 1000 μm;
and a mean pore size ranging from 1 to 100 nm.

39. The process according to claim 37, wherein
the solid support is suspended in a suspending solvent or a mixture of suspending solvents selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water;
the slurry thus obtained is applied to the separation means or to the separation column.

40. The process according to claim 35, wherein the composition after step iia), step iib) or step iic)
is dissolved or suspended in a diluting solvent or diluting solvent mixture selected from the group consisting of aliphatic hydrocarbons, aromatic hydrocarbons, halogenated hydrocarbons, carboxylic acids, esters, alcohols, ethers, ketones, acetals, ketals, nitriles, dimethyl sulfoxide, formamide, dimethylformamide and water, and
the diluted composition thus obtained is subjected to step iii).

41. The process according to claim 35, wherein
iii) after applying the composition of step iia), iib) or step iic) onto the separation means, the diameter of the surface of said separation means being larger than the height of said separation means or after applying the composition of step iia), iib) or step iic) onto the separation column;
iiia) one elutes impurities and by-products with a mixture of a non-polar and a polar solvent having a volumetric ratio ranging from 90:10 to 99:1;
iiib) one elutes the product with a mixture of a non-polar and a polar solvent having a volumetric ratio ranging from 60:40 to 85:15;
iv) optionally one subjects the remainder from step iiib) to a further distillation
or,
iii) after applying the composition of step iia), iib) or step iic) onto the separation means, the diameter of the surface of said separation means being larger than the height of said separation means or
after applying the composition of step iia), iib) or step iic) onto the separation column;
iiia) one elutes the product with a mixture of a non-polar and a polar solvent having a volumetric ratio ranging from 60:40 to 85:15;
iiib) one elutes impurities and by-products with a mixture of a non-polar and a polar solvent having a volumetric ratio ranging from 90:10 to 99:1;
iv) optionally one subjects the remainder from step iiia) to a further distillation.

42. The process according to claim 41, wherein
the non-polar solvent is at least one of heptane or cyclohexane,
the polar solvent is at least one of isopropylacetate or ethylacetate and
the mixture of the non-polar solvent and the polar solvent comprises at least one polar solvent and at least one non-polar solvent.

43. Quinone preparation obtained by the process according to claim 35 comprising:
A) 90 to 100 w % of quinone (C30) 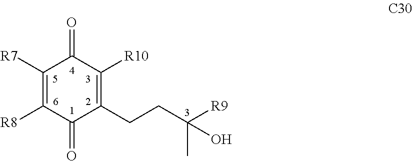
with R7, R8, R10 being H or CH₃; R9 being alkyl, alkenyl;
B) 0,0001 to 9999/1000 ppm of Cu;
C) 0,0001 to 100 ppm of organic chlorine;
D) minor components
with minor components being all chemical entities besides those mentioned under A), B) and C) which at most amount up to 10 w % minus the amount of components B) and C), and with the sum of A) to D) not exceeding 100 w %.

44. An animal nutrition composition, dietary supplement, or beverage additive comprising the quinone preparation according to claim 43.
